(19) United States
(12) Patent Application Publication
(10) Pub. No.: US 2019/0177279 A1
(43) Pub. Date: Jun. 13, 2019

(54) CARBOXYLIC ACID AROMATIC 1,2-CYCLOPROPYLAMIDES

(71) Applicant: Bayer Pharma Aktiengesellschaft, Berlin (DE)

(72) Inventors: Stefan Bäurle, Berlin (DE); Jens Nagel, Daxweiler (DE); Andrea Rotgeri, Berlin (DE); Adam James Davenport, Abingdon (GB); Christopher Charles Stimson, Abingdon (GB)

(21) Appl. No.: 16/211,486

(22) Filed: Dec. 6, 2018

(30) Foreign Application Priority Data
Dec. 13, 2017 (EP) 17207111.0

(51) Int. Cl. C07D 231/12; C07D 213/55; A61P 29/00

(52) U.S. Cl. CPC C07D 231/12; A61P 29/00; C07D 213/55

(57) ABSTRACT

The present invention relates to carboxylic acid aromatic 1,2-cyclopropylamides of general formula (I) as described and defined herein, to pharmaceutical compositions and combinations comprising said compounds and to the use of said compounds for manufacturing a pharmaceutical composition for the treatment or prophylaxis of a disease, in particular of neurogenic disorder, as a sole agent or in combination with other active ingredients.

The present invention relates to carboxylic acid aromatic 1,2-cyclopropylamides of general formula (I) as described and defined herein, to pharmacological compositions and combinations comprising said compounds and to the use of said compounds for manufacturing a pharmaceutical composition for the treatment or prophylaxis of a disease, in particular of Bradykinin B1 receptor associated disorders which are related to inflammation or at least partially driven by neurogenic events like diseases related to chronic pain or frequent pain conditions like but not restricted to osteoarthritis, rheumatoid arthritis, gout, inflammatory bowel disease, and endometriosis and diseases related to Bradykinin B1 receptor activation and/or up-regulation in affected tissue like but not restricted to asthma, fibrosis in various tissues or diabetes as a sole agent or in combination with other active ingredients.

### BACKGROUND OF THE INVENTION

The present invention relates to chemical compounds that antagonize the effects of human Bradykinin B1 receptor (Gene Name BDKRB1, Gene ID 623).

The Bradykinin B1 receptor is a membrane-bound G-protein coupled receptor, which is linked to a second messenger system that triggers increase of intracellular calcium concentrations. The main signalling pathway is linked to Gq protein and phospholipase C (Leeb-Lundberg, L. M. et al. (2005), Pharmacol Rev 57(1): 27-77). Activation of Bradykinin B1 receptor has been shown to be pro-algesic, pro-fibrotic, and proinflammatory while Bradykinin B1 receptor antagonists had clear anti-inflammatory and analgesic effects in various animal models (Gougat, J. B. et al. (2004), J Pharmacol Exp Ther 309(2): 661-669; Dias, J. P. et al. (2007), Br J Pharmacol 152(2): 280-287; Schuelert, N. et al. (2015), Eur J Pain 19(1): 132-142). As consequence of Bradykinin B1 receptor activity increased gene expression and protein levels of proinflammatory cytokines like e.g. 11-6 and 11-8 that attract and activate inflammatory leucocytes, increase of PGE2 (Prostaglandin 2) levels and therefore activation of the inflammation related prostaglandin pathway, phosphorylation and upregulation of TRPV1 (Transient Receptor Potential Vanilloid 1) receptors which are important mediators of pain transduction and induction of neurogenic inflammation (neuropeptide release in inflamed tissue) were observed (Phagoo, S. B. et al. (1999). Mol Pharmacol 56(2): 325-333; Westermann, D. et al. (2009), Diabetes 58(6): 1373-1381; Walsh, D. A. et al. (2006), Curr Drug Targets 7(8): 1031-1042; Farkas S. et al. (2011), Drugs of the Future 36(4): 301-319).

Bradykinin B1 receptor agonists are endogenously produced by the activated kallikreing-kinin system. This system consists of circulating kininogens, the ubiquitous expressed proteolytic enzymes kallikreins which are activated by tissue damage, and kinins which are formed by activated kallikreins out of kininogens (Review Fincham, C. I. et al. (2009), Expert Opin Ther Pat 19(7): 919-941). These kinins (e.g. bradykinin, kalidin, des-Arg9-bradykinin, des-Arg10-kalidin) are proinflammatory peptides that mediate vascular and pain responses to tissue injury, with functions in cardiovascular homeostasis, contraction or relaxation of smooth muscle, inflammation and nociception. They exert most of their effects by interacting with two classes of G-protein-coupled receptors called Bradykinin receptor 1 and 2. The classification of the kinin receptors was originally achieved by means of pharmacological studies carried out at the end of the 1970s. During the 1990s, the existence of Bradykinin B1 receptor and B2 receptors was further confirmed through cloning and genetic deletion studies (Menke, J. G. et al. (1994), J Biol Chem 269(34): 21583-21586). The past 30 years of research on the kinin system has indicated that both Bradykinin B1 receptor and B2 receptor are involved in pain and inflammation (Leeb-Lundberg, L. M. et al. (2005), Pharmacol Rev 57(1): 27-77; Marceau, F. (2005), Trends Pharmacol Sci 26(3): 116-118; Marceau, F. (2004), Nat Rev Drug Discov 3(10): 845-852; Chen, J. J. et al. (2007), Expert Opin Ther Targets 11(1): 21-35).

It has been demonstrated that the B2 receptor is widely expressed in a constitutive manner throughout most mammalian tissues. In contrast, the Bradykinin B1 receptor is not constitutively expressed to a great extent under normal conditions, but is up-regulated under various inflammatory conditions such as asthma, arthritis and osteoarthritis, sepsis and type-1 diabetes, as well as by some neuropathological diseases such as epilepsy, stroke and multiple sclerosis. Bradykinin B1 receptor up-regulation can be induced for example by Il-1beta (Phagoo, S. B. et al. (1999), Mol Pharmacol 56(2): 325-333) and Bradykinin B2 receptor activation (NF-kB activation leading to IL-1β expression in fibroblasts) (Leeb-Lundberg, L. M. et al. (2005), Pharmacol Rev 57(1): 27-77).

Once upregulated, the Bradykinin B1 receptor is expressed on neurons, macrophages, neutrophils, fibroblasts, smooth muscle cells and the vascular endothelium (Fincham, C. I. et al. (2009), Expert Opin Ther Pat 19(7): 919-941). Recent findings suggest that the Bradykinin B1 receptor expressed in the peripheral and in the central nervous system is involved in processing of inflammatory pain (Schuelert, N. et al. (2015). Eur J Pain 19(1): 132-142).

In contrast to Bradykinin B2 receptor and many other GPCRs (G protein-coupled receptors), the Bradykinin B1 receptor does not show agonist induced internalization or relevant desensitization (Prado, G. N. et al. (2002), J Cell Physiol 193(3): 275-286; Eisenbarth, H. et al. (2004), Pain 110(1-2): 197-204). Activation of Bradykinin B1 receptor triggers auto-induction of the receptor. This might lead to an augmentation of the inflammatory or pain-inducing processes.

Therefore, Bradykinin B1 receptor has been suggested to have a pivotal role including but not limited to several chronic diseases involving diabetes, fibrosis, inflammation, neuroinflammation, neurodegeneration, inflammatory pain, and neuropathic pain (Campos, M. M. et al. (2006), Trends Pharmacol Sci 27(12): 646-651; Wang, P. H. et al. (2009), Int Immunopharmacol 9(6): 653-657; Passos, G. F. et al. (2013), Am J Pathol 182(5): 1740-1749; Gobeil, F. et al. (2014), Peptides 52: 82-89; Huart, A. (2015), Front Pharmacol 6: 8). The contribution of Bradykinin B1 receptor activation in inflammation and pain processes is supported by the demonstration that Bradykinin B1 receptor knockout mice have a largely decreased response to nociceptive and proinflammatory stimuli (Ferreira, J. et al. (2001), Neuropharmacology 41(8): 1006-1012; Ferreira, J. et al. (2005), J Neurosci 25(9): 2405-2412). The therapeutic impact of Bradykinin B1 receptor blockage for inflammation related diseases is supported further by the pharmacological properties of Bradykinin B1 receptor antagonists shown in many inflammatory and neuropathic pain models (Gougat, J. B. et al. (2004), J Pharmacol Exp Ther 309(2): 661-669; Fox, A. et al. (2005), Br J Pharmacol 144(7): 889-899).

The fact that Bradykinin B1 receptor expression is induced under disease conditions clearly raises the possibility that therapeutic use of Bradykinin B1 receptor antagonists should be devoid of undesired adverse effects. This property supports the suitability of Bradykinin B1 receptor antagonists for treatment of benign diseases like endometriosis due the expected positive risk benefit ratio. The patient populations for nociceptive pain and neuropathic pain are large, and are driven by separate disease trends that necessitate pain relief. Chronic pain of moderate to severe intensity occurs in 19% of adult Europeans, seriously affecting the quality of their social and working lives (Breivik et al., Eur J Pain. 2006 May; 10(4):287-333). Unfortunately, current treatments for pain are only partially effective, and many cause life-style altering, debilitating, and/or dangerous side effects. For example, non-steroidal anti-inflammatory drugs (NSAIDS) such as aspirin, ibuprofen, and indomethacin are moderately effective against inflammatory pain but they are also renally toxic, and high doses tend to cause gastrointestinal irritation, ulceration, bleeding, confusion and increased cardiovascular risk. Notably, Vioxx was withdrawn from the market in 2004 due to a risk of myocardial infarction and stroke. Patients treated with opioids frequently experience confusion and constipation, and long-term opioid use is associated with tolerance and addiction. Local anaesthetics such as lidocaine and mexiletine simultaneously inhibit pain and cause loss of normal sensation. In addition, when used systemically, local anaesthetics are associated with adverse cardiovascular effects. Thus, there is currently an unmet need in the treatment of chronic pain in general.

Especially in gynaecological therapy field, endometriosis is the diseases associated with chronic pelvic pain severely affecting quality of life of the patients. Globally, approximately 11% of women aged 15-49 years are affected by endometriosis and additional 6% of women suffer from symptoms suggestive for endometriosis. Main symptoms of endometriosis are chronic or frequent pelvic pain, dyspareunia, dyschezia, dysuria and sub- or infertility. These symptoms severely impair quality of life of patients. Diagnosis of the disease involves a complete medical history, a physical examination and a laparoscopy. As an ultimate confirmation of endometriosis can only be made invasively and symptoms are often unspecific, the mean time from initial symptoms to diagnosis of endometriosis is about 7-10 years. Therefore, endometriosis is under-diagnosed and the number of affected women might be much higher than anticipated. Recently published EndoCost study demonstrated that cost of productivity loss of €6,298 per woman were double the healthcare cost of €3,113 per women, driven mainly by surgery and monitoring visit (Gao, X. et al. (2006), Fertil Steril 86(6): 1561-1572; Simoens S, et al. Hum Reprod (2012), 27(5):1292-9; De Graaff A, et al. (2013), Hum Reprod; 28(10): 2677-85).

Endometriosis is characterized by growth of endometrial tissue outside of the uterine cavity forming benign tumours (lesions) in the affected part of the body. Depending on lesion location and innervation severity of pain symptoms is observed. Up-regulation of various inflammation markers observed in the affected tissue and in the peritoneal tissue underline the inflammatory character of the disease (Stratton, P. et al. (2011), Hum Reprod Update 17(3): 327-346; Gao, X. et al. (2006), Fertil Steril 86(6): 1561-1572; Laux-Biehlmann et al. (2015), Trends Pharmacol Sci 36(5):270-276).

The Bradykinin B1 receptor was identified in endometriosis lesion by immune-histological-chemical (IHC) staining (Yoshino et al. Journal of Reproductive Immunology 112 (2015) 121-140; www.proteinatlas.org) and analysis of mRNA expression of Bradykinin B1 receptor in affected tissue shows a positive correlation to pain severity reported by endometriosis patients. Data describing a role of Bradykinin B1 receptors in affecting the outcome of an endometriosis mouse model (Jingwei, C. et al. (2015), J Tradit Chin Med 35(2): 184-191) further support the concept to treat endometriosis with Bradykinin B1 receptor antagonists.

Suspected endometriosis is initially treated with non-steroidal anti-inflammatory drugs (NSAID) or combined oral contraceptives (COC) which are used off label. This procedure delays endometriosis diagnosis. Laparoscopy is the gold standard for endometriosis diagnosis which is performed when the initial treatment options fail. During laparoscopy, endometriotic lesions are ablated. However, this procedure is accompanied by a high recurrence rate. Approximately, 70% of treated patients have persistent symptoms that are not managed. Currently, there is no long-term medication available in COC/P (Combined Oral Contraceptives/Progestin) non-responder endometriosis patients in which COCs and progestins failed. Treatment with Gonadotropin Releasing Hormone (GnRH) agonists, which are used as second line therapy (without proof of being superior versus first line) are only approved for short-term treatment (6 months). After GnRH agonist application, systemic estradiol levels are suppressed up to 90% leading to chemical castration with menopausal side effects like bone mass loss and hot flushes. Therefore, new and long-term treatment options with reduced side-effects and high efficacy for endometriosis patients, in particular for patients with COC/P non-responder endometriosis, are urgently needed.

On this background the Bradykinin B1 receptor antagonists are of value for treatment of disorders which are related to inflammation or at least partially driven by neurogenic events like diseases related to chronic pain or frequent pain conditions like but not restricted to osteoarthritis (Kaufman, G. N. et al. (2011), Arthritis Res Ther 13(3): R76), rheumatoid arthritis (Cassim, B. et al. (2009), Rheumatology 48(5): 490-496), gout (Silva, C. R. et al. (2016), Ann Rheum Dis 75(1): 260-268), burn injuries and sunburn (Eisenbarth, H. et al. (2004), Pain 110(1-2): 197-204), inflammatory bowel disease, endometriosis (Yoshino et al. Journal of Reproductive Immunology 112 (2015) 121-140; Laux-Biehlmann et al. (2015), Trends Pharmacol Sci 36(5): 270-276; Jingwei, C. et al. (2015), J Tradit Chin Med 35(2): 184-191), pre-eclampsia (Moyes, A. J. et al. (2014), Hypertens Pregnancy 33(2): 177-190), diabetic neuropathy (Dias, J. P. et al. (2007), Br J Pharmacol 152(2): 280-287) including neuropathy related to diabetes type 1 and diabetes type 2, cardiac inflammation (Westermann, D. et al. (2009), Diabetes 58(6): 1373-1381), renal inflammation (Bascands, J. et al. (2009), Biochem Biophys Res Commun 386(2): 407-412), pancreatitis and diseases related to Bradykinin B1 receptor activation and/or up-regulation in affected tissue like but not restricted to asthma and cough (Bertram, C. M. et al. (2009), J Leukoc Biol 85(3): 544-552), atherosclerosis, diabetes (Dias, J. P. et al. (2012), J Cardiovasc Pharmacol 60(1): 61-69), adipositas including metabolic syndrome (Dias, J. P. et al. (2012), Diabetes Obes Metab 14(3): 244-253), diseases related to muscle atrophy including cachexia (Parreiras, E. S. L. T. et al. (2014), Clin Sci 127(3): 185-194) not limited to cancer cachexia, neuropathic pain (Luiz, A. P. et al. (2015), Neuroscience 300: 189-200), pruritus or itch (Hosogi, M. et al. (2006), Pain 126(1-3): 16-23), cancer (da Costa, P. L. et al. (2014), Cancer Lett 345(1): 27-38), neurodegenerative diseases such as amyotrophic lateral sclerosis (ALS) or Alzheimer's disease (Lacoste, et al. (2013) J Neuroinflammation 10: 57), fibrosis in cardiacs (Westermann, D. et al. (2009), Diabetes 58(6): 1373-1381), fibrosis in renal (Huart, A. et al. (2015), Front Pharmacol 6: 8) and fibrosis in lung tissues, overactive urinary bladder syndrome and cystitis (Forner, S. et al. (2012), Br J Pharmacol 167(8): 1737-1752 and Belichard, P. et al (1999), Br J Pharmacol 128(1):213-219), impaired or painful wound healing (Schremmer-Danninger, E. et al. (2004), Biol Chem 385(11): 1069-1076) and sepsis (Murugesan, P et al. (2016), J Infect Dis 213(4): 532-540).

Several Bradykinin B1 receptor antagonists are known from prior art (Expert Opinion on Therapeutic Patents (2012), 22:12, 1443-1452). Various approaches for finding new Bradykinin B1 receptor antagonists are described, in particular peptidic structures and small molecules. Especially, arylsulfonamides and so-called cyclopropyl-carboxamides as the two main types of small molecules were investigated during the last decade.

WO2003/065789 (Merck) discloses bradykinin B1 receptor antagonists or inverse agonists of the following general formula

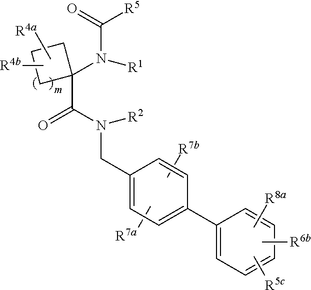

which are disclosed to be useful in the treatment or prevention of symptoms such as pain and inflammation associated with the bradykinin B1 pathway.

Merck was developing the bradykinin B1 receptor antagonist MK-0686 (structure shown below)

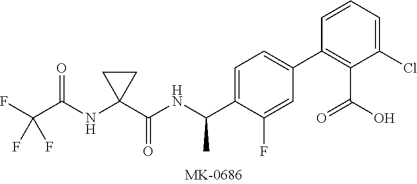 [SMILES: C[C@@H](NC(=O)C1(NC(=O)C(F)(F)F)CC1)c1ccc(-c2cccc(Cl)c2C(=O)O)cc1F]

for the potential treatment of pain and inflammation. Several phase II trials in subjects with osteoarthritis and with post-herpetic neuralgia were initiated. Merck accounted that the compound has a suboptimal pharmacokinetic profile due to metabolic liability.

Jerini AG, now Shire Group, investigated active Bradykinin B1 receptor antagonists, for example (see WO2009/036996)

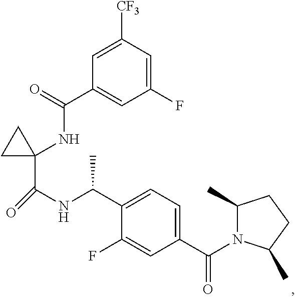 [SMILES: C[C@@H]1CC[C@H](C)N1C(=O)c1ccc([C@@H](C)NC(=O)C2(NC(=O)c3cc(F)cc(C(F)(F)F)c3)CC2)c(F)c1]

which was reported to have in addition to its activity and acceptable penetration profile reasonable aqueous solubility and pharmacokinetic profile in rat, whereas its human metabolic stability was still poor (Schaudt M, Locardi E, Zischinsky G, et al., Bioorg Med Chem Lett 2010; 20:1225-8). Jerini exchanged the cyclopropyl-carboxamide moiety to a semicarbazide or to a five-membered diamino-heterocyclic ring or even to hydroxyureas without any explanation.

Starting with arylsulfonamide compounds as Bradykinin B1 receptor antagonists, Boehringer Ingelheim reported several cyclopropyl-carboxamides out of their further development compounds like of the following structure

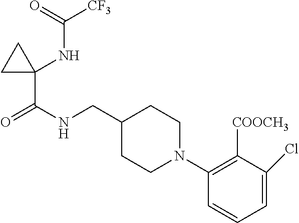 [SMILES: COC(=O)c1c(Cl)cccc1N1CCC(CNC(=O)C2(NC(=O)C(F)(F)F)CC2)CC1]

or related to that emerged with the highest binding affinity measured on human B1R-expressing CHO cells (Expert Opinion on Therapeutic Patents (2012), 22:12, 1443-1452).

In WO2012059776 Gedeon Richter reported about cyclopropyl-carboxamides of the following formula

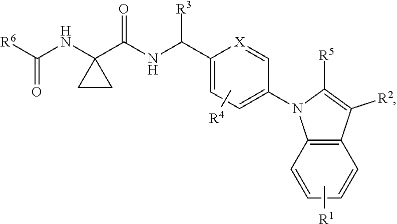

wherein R³ is selected from (1) —COOR; (2) —CN; (3) —CONR^{a}R^{b};

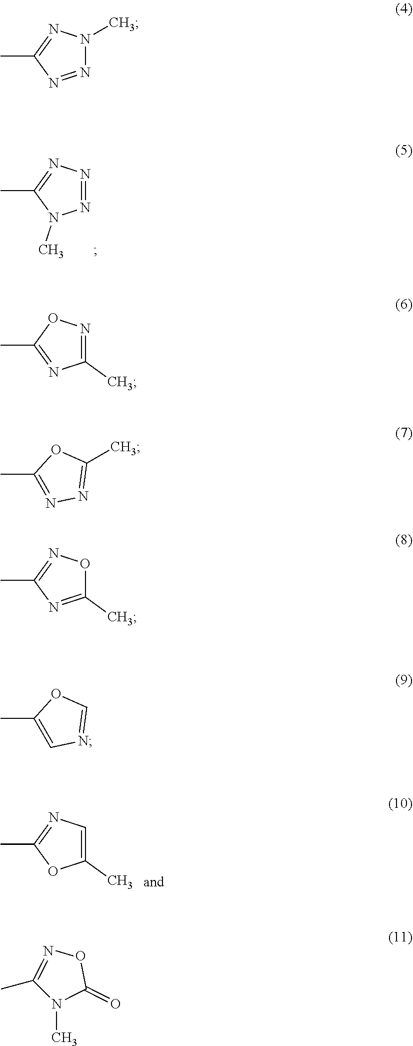

A majority of the compounds have a Kᵢ value below 20 nM on human recombinant Bradykinin receptors (expressed in CHO cells). Several indolyl compounds substituted with a tetrazol moiety are disclosed and represented by the following compound:

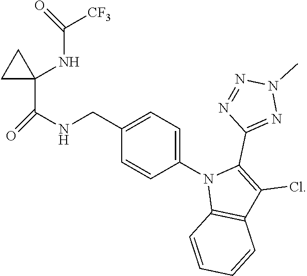 [SMILES: Cn1nnc(-c2c(Cl)c3ccccc3n2-c2ccc(CNC(=O)C3(NC(=O)C(F)(F)F)CC3)cc2)n1]

WO2005085227 (Smith Kline Beecham) discloses inhibitors of protein kinase B (PKB/Akt, PKB or Akt) of the formula

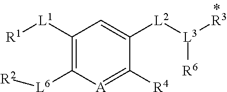

wherein

A is selected from: nitrogen, —C-halogen and —CH;

R¹ is selected from the group consisting of aryl, substituted aryl, cycloalkyl, substituted cycloalkyl, heterocycle and substituted heterocycle;

R² is selected from alkyl, substituted alkyl, cycloalkyl, substituted cycloalkyl, heterocycle, substituted heterocycle, and a cyclic or polycyclic aromatic ring,

L² is selected from the group consisting of a bond, —O—, heterocycle, —N(R⁵)—, —N(R⁵)C(O)—, —S—, —S(O)—, —S(O₂)—, and —C(O)N(R⁵)—; and

L¹ as well as L⁶ can be a bond, —O—, —N(R⁵)—, —S—, —S(O), —S(O₂)—, alkyl, and —N(R⁵)C(O)—. Neither L¹ nor L⁶ can be a heteroaryl or heterocyclic group. R⁴ is defined as hydrogen or halogen. The compounds are disclosed to be suitable for the treatment of cancer and arthritis. Tetrazole-substituted phenyl or pyridinyl compounds are not specifically disclosed.

In WO2012112567 (Georgetown University) small molecule inhibitors of ATP/GTP binding protein like 2 (AGBL2) of the formula

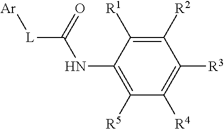

are disclosed wherein R² as well as R⁴ are each independently selected from hydrogen, halogen, hydroxyl, cyano, nitro, substituted or unsubstituted alkyl, substituted or unsubstituted heteroalkyl, substituted or unsubstituted amino, substituted or unsubstituted aryl, substituted or unsubstituted heteroaryl, substituted or unsubstituted alkoxyl, substituted or unsubstituted aryloxyl, substituted or unsubstituted carbonyl, or substituted or unsubstituted carboxyl.

The compounds are disclosed to be used in methods for treating or preventing cancer and neurologic disorders. A tetrazole moiety as substituent at the benzene core structure is not specifically disclosed.

WO2009005638 (Merck) discloses a class of pyridinyl and pyrimidinyl derivatives of the formula

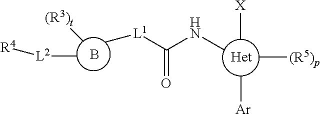

wherein the substituent Ar is aryl or heteroaryl, optionally substituted with halo, methyl, methoxy, halomethyl, amino, hydroxyl, C(O)OCH₃ or C(O)NHCH₃, X can be OH, SH or NH₂ and R⁵ is selected from H, OH, NH₂, nitro, CN, amide, carboxyl, C₁-C₇ alkoxy, C₁-C₇ alkyl, C₁-C₇ haloalkyl, C₁-C₇ haloalkyloxy, C₁-C₇ hydroxyalkyl, C₁-C₇ alkenyl, C₁-C₇ alkyl-C(═O)O—, C₁-C₇ alkyl-C(═O)—, C₁-C₇ alkynyl, halo, hydroxyalkoxy, C₁-C₇ alkyl-NHSO₂—, C₁-C₇ alkyl-S O₂NH—, C₁-C₇ alkylsulfonyl, C₁-C₇ alkylamino or di(C₁-C₇)alkylamino. Neither X nor R⁵ can be a heteroaryl or heterocyclic group. Tetrazolyl is not specifically disclosed as substituent Ar. The compounds are disclosed to be used to treat cancer.

WO 2016168059 (DOW Agrosciences LLC) discloses compounds containing a 1,2-cyclopropyl of formula one

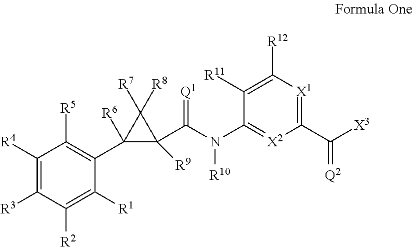

wherein Q² is S or O, and wherein X³ is selected from the group consisting of N(R¹⁵)(substituted or unsubstituted phenyl), N(R¹⁵) (substituted or unsubstituted heterocyclyl), and substituted or unsubstituted heterocyclyl. The compounds are disclosed as having pesticidal utility against pests in Phyla Arthropoda, Mollusca, and Nematoda. Furthermore processes to produce such compounds, intermediates used in such processes, pesticidal compositions containing the compounds, and processes of using such pesticidal compositions against such pests are also disclosed in WO 2016168059.

WO2012103583 (Bionomics) discloses 1,2-cyclopropyl-carboxamide compounds of formula (I)

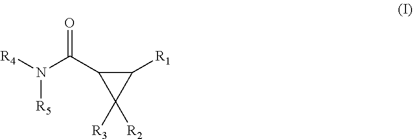

wherein R² is selected from C₁-C₄ alkyl, C₃-C₅ alkenyl, F, Br, Cl, CN, or C₁-C₄ haloalkyl; R⁴ is selected from optionally substituted heteroaryl, optionally substituted heterocyclyl, or optionally substituted aryl, and R⁵ is selected from hydrogen or optionally substituted alkyl. Such compounds are disclosed to be useful in the positive modulation of the alpha 7 nicotinic acetylcholine receptor (α7nAChR). The disclosure of WO2012103583 also relates to the use of these compounds in the treatment or prevention of a broad range of diseases in which the positive modulation of α7nAChR is advantageous, including neurodegenerative and neuropsychiatric diseases and inflammatory diseases.

WO2007087066 (Vertex) discloses compounds and pharmaceutically acceptable compositions thereof, which are disclosed to be useful as modulators of ATP-Binding Cassette (“ABC”) transporters or fragments thereof, including Cystic Fibrosis Transmembrane Conductance Regulator (“CFTR”), having a benzamide core structure (I)

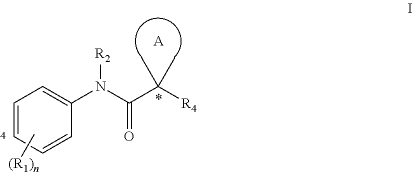

wherein ring A is an optionally substituted cycloaliphatic or an optionally substituted heterocycloaliphatic where the atoms of ring A adjacent to C* are carbon atoms. R₄ is an optionally substituted aryl or an optionally substituted heteroaryl. R¹ is independently an optionally substituted C₁-C₆ aliphatic, an optionally substituted aryl, an optionally substituted heteroaryl, an optionally substituted C₃-C₁₀ membered cycloaliphatic or an optionally substituted 4 to 10 membered heterocycloaliphatic, carboxy, amido, amino, halo, or hydroxy, provided that at least one R¹ is an optionally substituted aryl or an optionally substituted heteroaryl and said R¹ is attached to the 3- or 4-position of the phenyl ring. Compounds in which the phenyl ring of the benzamide core structure is substituted with tetrazolyl are not disclosed.

So, the state of the art described above does not describe the specific compounds of general formula (I) of the present invention containing a carboxylic acid aromatic 1,2-cyclopropylamide moiety as defined herein or an isomer, enantiomer, diastereomer, racemate, hydrate, solvate, or a salt thereof, or a mixture of same, as described and defined herein, and as hereinafter referred to as “compounds of the present invention”, or their pharmacological activity.

### SUMMARY OF THE INVENTION

The present invention covers carboxylic acid aromatic amides of general formula (I):

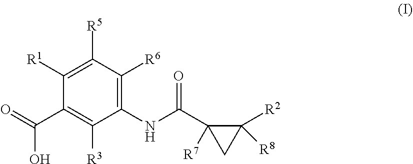

in which
R¹ represents
   phenyl,
   5- or 6-membered heteroaryl, wherein said 5-membered heteroaryl contains 1, 2 or 3 heteroatoms or heteroatom-containing groups independently selected from the group consisting of S, N, NH, and O, and wherein said 6-membered heteroaryl contains 1 or 2 nitrogen atoms, or
   bicyclic 8- to 10-membered heteroaryl containing 1, 2 or 3 heteroatoms or heteroatom-containing groups independently selected from NH, N, O, S, SO and SO₂,
   wherein said R¹ is optionally substituted at one or more carbon atoms with 1 to 3 substituents R^{1a} which are the same or different, wherein R^{1a} represents C₁-C₅-alkyl, C₃-C₇-cycloalkyl, —(C₁-C₃-alkyl)-(C₃-C₇-cycloalkyl), —OC₁-C₅-alkyl, —OC₃-C₇-cycloalkyl, NHR⁴, N(R⁴)₂, NH(C₃-C₇-cycloalkyl), halogen, CN, NHSO₂R⁴, SO₂R⁴, 5-to 7-membered lactam, or 4- to 7-membered heterocycloalkyl containing 1 or 2 heteroatoms or heteroatom-containing groups selected from NH, —NR⁴, N, O, S, SO and SO₂, and
   wherein independently, if R¹ represents 5-membered heteroaryl or bicyclic 8- to 10-membered heteroaryl, each ring nitrogen atom, if present, of said R¹ is optionally substituted with a substituent R^{1b}, wherein R^{1b} represents C₁-C₅-alkyl, —(C₁-C₃-alkyl)-(C₃-C₇-cycloalkyl), C₃-C₇-cycloalkyl, SO₂R⁴, or 4- to 7-membered heterocycloalkyl containing 1 or 2 heteroatoms or heteroatom-containing groups selected from NH, —NR⁴, N, O, S, SO and SO₂, and
   if R₁ₐ represents C₁-C₅-alkyl, C₃-C₇-cycloalkyl, —(C₁-C₃-alkyl)-(C₃-C₇-cycloalkyl), —OC₁-C₅-alkyl or —OC₃-C₇-cycloalkyl and/or if R^{1b} represents C₁-C₅-alkyl, —(C₁-C₃-alkyl)-(C₃-C₇-cycloalkyl) or C₃-C₇-cycloalkyl, said C₁-C₅-alkyl, C₃-C₇-cycloalkyl, —(C₁-C₃-alkyl)-(C₃-C₇-cycloalkyl), —OC₁-C₅-alkyl and —OC₃-C₇-cycloalkyl independently are optionally substituted with one or more substituents independently selected from the group consisting of methyl, ethyl, OH, OR⁴ and F, and
   if R^{1a} and/or R^{1b} represent 4- to 7-membered heterocycloalkyl, each carbon atom of said 4- to 7-membered heterocycloalkyl is optionally substituted with one or more substituents independently selected from the group consisting of OH, OR⁴ and F;
R² represents
   —(CH₂)ₚ—(C₅-C₇-cycloalkyl),
   —(CH₂)ₚ-phenyl,
   5- or 6-membered heteroaryl wherein said 5-membered heteroaryl contains 1, 2 or 3 heteroatoms or heteroatom-containing groups independently selected from the group consisting of S, N, NH, and O, and wherein said 6-membered heteroaryl contains 1 or 2 N, or
   bicyclic 8- to 10-membered heteroaryl, containing 1, 2 or 3 heteroatoms or heteroatom-containing groups independently selected from NH, N, O, S, SO and SO₂,
   wherein said R² is optionally substituted at one or more carbon atoms with 1 to 3 substituents R^{2a} which are the same or different wherein R^{2a} represents C₁-C₅-alkyl, C₃-C₇-cycloalkyl, —(C₁-C₃-alkyl)-(C₃-C₇-cycloalkyl), —OC₁-C₅-alkyl, —OC₃-C₇-cycloalkyl, halogen, OH or CN, and
   wherein independently, if R² represents 5-membered heteroaryl or bicyclic 8- to 10-membered heteroaryl, each ring nitrogen atom, if present, of said R² is optionally substituted with a substituent R^{2b} wherein R^{2b} represents C₁-C₅-alkyl, C₃-C₇-cycloalkyl or —(C₁-C₃-alkyl)-(C₃-C₇-cycloalkyl), and
   if R^{2a} represents C₁-C₅-alkyl, C₃-C₇-cycloalkyl, —(C₁-C₃-alkyl)-(C₃-C₇-cycloalkyl), —OC₁-C₅-alkyl or —OC₃-C₇-cycloalkyl and/or if R^{2b} represents C₁-C₅-alkyl, C₃-C₇-cycloalkyl or —(C₁-C₃-alkyl)-(C₃-C₇-cycloalkyl), said C₁-C₅-alkyl, C₃-C₇-cycloalkyl, —(C₁-C₃-alkyl)-(C₃-C₇-cycloalkyl), —OC₁-C₅-alkyl and —OC₃-C₇-cycloalkyl independently are optionally substituted with one or more substituents independently selected from the group consisting of OH, OR⁴, and 1 to 5 fluorine atoms;
p 0 or 1;
R³ represents H or F;
R⁴ represents C₁-C₅-alkyl, optionally substituted with 1 to 5 fluorine atoms;
R⁵ represents H, halogen, CN, C₁-C₅-alkyl, or —OC₁-C₅-alkyl wherein said C₁-C₅-alkyl and —OC₁-C₅-alkyl are optionally substituted with 1 to 5 fluorine atoms; and
R⁶ represents H, halogen, CN, OH, C₁-C₅-alkyl, or —OC₁-C₅-alkyl wherein said C₁-C₅-alkyl and —OC₁-C₅-alkyl are optionally substituted with 1 to 5 fluorine atoms; and
R⁷ and R⁸ independently represent H, or C₁-C₃-alkyl, wherein the C₁-C₃-alkyl is independently optionally substituted with 1 to 3 fluorine atoms;

or an isomer, enantiomer, diastereomer, racemate, hydrate, solvate, or a salt thereof, or a mixture of the same.

The present invention further relates to pharmaceutical compositions and combinations comprising said compounds, to the use of said compounds for manufacturing a medicament for the treatment or prophylaxis of diseases or disorders and for the treatment of pains, which are associated with such diseases as well as for the treatment of inflammation, which are associated with such diseases; Furthermore, the present invention relates to pharmaceutical compositions and combinations comprising said compounds, to the use of said compounds for the treatment or prophylaxis of diseases or disorders and for the treatment of pains, which are associated with such diseases as well as for the treatment of inflammation, which are associated with such diseases.

It has now been found, and this constitutes the basis of the present invention, that said compounds of the present invention have surprising and advantageous properties. In particular, said compounds of the present invention have surprisingly been found to effectively inhibit Bradykinin B1 receptor. Hence, the invention particularly relates to said compounds for use in the treatment or prophylaxis of following diseases or disorders:

Pain and inflammation, in particular any one of
visceral pain e.g. related to pancreatitis, interstitial cystitis, renal colic, or prostatitis, chronic pelvic pain, or pain related to infiltrating endometriosis;
neuropathic pain such as post herpetic neuralgia, acute zoster pain, pain related to nerve injury, the dynias, including vulvodynia, phantom limb pain, pain related to root avulsions, pain related to radiculopathy, painful traumatic mononeuropathy, painful entrapment neuropathy, pain related to carpal tunnel syndrome, ulnar neuropathy, pain related to tarsal tunnel syndrome, painful diabetic neuropathy, painful polyneuropathy, trigeminal neuralgia, or pain related to familial amyloid polyneuropathy;
central pain syndromes potentially caused by virtually any lesion at any level of the nervous system including but not limited to pain related to stroke, multiple sclerosis, and spinal cord injury; and
postsurgical pain syndromes (including postmastectomy pain syndrome, postthoracotomy pain syndrome, stump pain), bone and joint pain (osteoarthritis), spine pain (including acute and chronic low back pain, neck pain, pain related to spinal stenosis), shoulder pain, repetitive motion pain, dental pain, pain related to sore throat, cancer pain, burn pain including sun-burn, myofascial pain (pain related to muscular injury, fibromyalgia) postoperative and perioperative pain (including but not limited to general surgery, orthopaedic, and gynaecological surgery); and
acute and chronic pain, chronic pelvic pain, endometriosis associated pain, dysmenorrhea associated pain (primary and secondary), pain associated with uterine fibroids, vulvodynia associated pain, as well as pain associated with angina, or inflammatory pain of varied origins (including but not limited to pain associated with osteoarthritis, rheumatoid arthritis, rheumatic disease, tenosynovitis, gout, ankylosing spondylitis, and bursitis);
and diseases selected from or related to any one of:
gynaecological disorders and/or diseases, or effects and/or symptoms which negatively influence women health including endometriosis, uterine fibroids, pre-eclampsia, hormonal deficiency, spasms of the uterus, or heavy menstrual bleeding;
the respiratory or excretion system including any of inflammatory hyperreactive airways, inflammatory events associated with airways disease like chronic obstructive pulmonary disease, asthma including allergic asthma (atopic or non-atopic) as well as exercise-induced bronchoconstriction, occupational asthma, viral or bacterial exacerbation of asthma, other non-allergic asthmas and wheezy-infant syndrome, chronic obstructive pulmonary disease including emphysema, adult respiratory distress syndrome, bronchitis, pneumonia, cough, lung injury, lung fibrosis, allergic rhinitis (seasonal and perennial), vasomotor rhinitis, angioedema (including hereditary angioedema and drug-induced angioedema including that caused by angiotensin converting enzyme (ACE) or ACE/neutral endopeptidase inhibitors like omepatrilat), pneumoconiosis, including aluminosis, anthracosis, asbestosis, chalicosis, ptilosis, siderosis, silicosis, tabacosis and byssinosis, bowel disease including Crohn's disease and ulcerative colitis, irritable bowel syndrome, pancreatitis, nephritis, cystitis (interstitial cystitis), kidney fibrosis, kidney failure, hyperactive bladder, and overactive bladder;
dermatology including pruritus, itch, inflammatory skin disorders including psoriasis, eczema, and atopic dermatitis;
affection of the joints or bones including rheumatoid arthritis, gout, osteoporosis, osteoarthritis, and ankylosing spondylitis;
affection of the central and peripheral nervous system including neurodegenerative diseases including Parkinson's and Alzheimer's disease, amyotrophic lateral sclerosis (ALS), epilepsy, dementia, headache including cluster headache, migraine including prophylactic and acute use, stroke, closed head trauma, and multiple sclerosis;
infection including HIV infection, and tuberculosis;
trauma associated with oedema including cerebral oedema, burns, sunburns, and sprains or fracture;
poisoning including aluminosis, anthracosis, asbestosis, chalicosis, ptilosis, siderosis, silicosis, tabacosis, and byssinosis uveitis;
diabetes cluster or metabolism like diabetes type 1, diabetes type 2, diabetic vasculopathy, diabetic neuropathy, diabetic retinopathy, post capillary resistance or diabetic symptoms associated with insulitis (e.g. hyperglycaemia, diuresis, proteinuria and increased nitrite and kallikrein urinary excretion), diabetic macular oedema, metabolic syndrome, insulin resistance, obesity, fat or muscle metabolism;
cachexia associated with or induced by any of cancer, AIDS, coeliac disease, chronic obstructive pulmonary disease, multiple sclerosis, rheumatoid arthritis, congestive heart failure, tuberculosis, familial amyloid polyneuropathy, mercury poisoning (acrodynia), and hormonal deficiency;
cardio-vascular system including congestive heart failure, atherosclerosis, congestive heart failure, myocardial infarct, and heart fibrosis; and
other conditions including septic shock, sepsis, muscle atrophy, spasms of the gastrointestinal tract, benign prostatic hyperplasia, and liver diseases such as non-alcoholic and alcoholic fatty liver disease, non-alcoholic and alcoholic steatohepatitis, liver fibrosis, or liver cirrhosis.

Additionally, compounds of the present invention reduce the release of inflammation related cytokines like IL-6 and IL-8. Hence, the present invention also relates to a method for reducing inflammation related cytokine production, the method comprising the step of administering an effective amount of a compound of the present invention to a patient in need thereof. The invention also relates to the compounds of the invention as defined herein for use in the treatment of a disease associated with increased release of inflammation related cytokines, preferably associated with increased release of IL-6 and/or IL-8.

### DETAILED DESCRIPTION OF THE INVENTION

The term “substituted” means that one or more hydrogen atoms on the designated atom or group are replaced with a selection from the indicated group, provided that the designated atom's normal valency under the existing circumstances is not exceeded. Combinations of substituents and/or variables are permissible.

The term “optionally substituted” means that the number of substituents can be equal to or different from zero. Unless otherwise indicated, it is possible that optionally substituted groups are substituted with as many optional substituents as can be accommodated by replacing a hydrogen atom with a non-hydrogen substituent on any available carbon or nitrogen or sulfur atom. Commonly, it is possible for the number of optional substituents, when present, to be 1, 2, 3, 4 or 5, in particular 1, 2 or 3.

As used herein, the term “one or more”, e.g. in the definition of the substituents of the compounds of general formula (I) of the present invention, means “one or a plurality up to the maximum possible amount”, e.g. if the term refers to the carbon atoms of a C₇-cycloalkyl, it relates to “1, 2, 3, 4, 5, 6 or 7”. In particular, “one or more” means “1, 2, 3, 4 or 5, particularly 1, 2, 3 or 4, more particularly 1, 2 or 3, even more particularly 1 or 2”.

When groups in the compounds according to the invention are substituted, it is possible for said groups to be mono-substituted or poly-substituted with substituent(s), unless otherwise specified. Within the scope of the present invention, the meanings of all groups, which occur repeatedly, are independent from one another. It is possible that groups in the compounds according to the invention are substituted with one, two or three identical or different substituents, particularly with one substituent.

The term “comprising” when used in the specification includes but is not restricted to “consisting of”.

The terms as mentioned in the present text have preferably the following meanings:

The term “halogen atom”, “halogen”, “halo-” or “Hal-” is to be understood as meaning a fluorine, chlorine, bromine or iodine atom, preferably a fluorine or a chlorine atom.

The term “C₁-C₅-alkyl” means a linear or branched, saturated, monovalent hydrocarbon group having 1, 2, 3, 4 or 5 carbon atoms, e.g. a methyl, ethyl, propyl, isopropyl, butyl, sec-butyl, isobutyl, tert-butyl, pentyl, isopentyl, 2-methylbutyl, 1-methylbutyl, 1-ethylpropyl, 1,2-dimethylpropyl, neo-pentyl or 1,1-dimethylpropyl group, or an isomer thereof.

The term “C₁-C₃-alkyl” means a linear or branched, saturated, monovalent hydrocarbon group having 1, 2 or 3 carbon atoms (“C₁-C₃-alkyl”), e.g. a methyl, ethyl, n-propyl or isopropyl group.

The term “—OC₁-C₅-alkyl” means a linear or branched, saturated, monovalent group which is attached through an oxygen atom, and in which the term “C₁-C₅-alkyl” is as defined supra, e.g. a methoxy, ethoxy, n-propoxy, isopropoxy, n-butoxy, sec-butoxy, isobutoxy, tert-butoxy, pentyloxy or isopentyloxy, or an isomer thereof. The hyphen at the beginning of the group indicates the point of attachment of said OC₁-C₅-alkyl group to the rest of the molecule. “C₃-C₇-cycloalkyl” is to be understood as meaning a saturated, monovalent, monocyclic or bicyclic hydrocarbon ring, which contains 3, 4, 5, 6 or 7 carbon atoms. Said C₃-C₇-cycloalkyl group is for example a monocyclic hydrocarbon ring, e.g. a cyclopropyl, cyclobutyl, cyclopentyl, cyclohexyl or cycloheptyl group, or a bicyclic hydrocarbon ring, e.g. a bicyclo[2.2.1]heptanyl or bicyclo[3.2.0]heptanyl group. Particularly, said ring contains 3, 4 or 5 carbon atoms (“C₃-C₅-cycloalkyl”) or 5, 6 or 7 carbon atoms (“C₅-C₇-cycloalkyl”).

The term “bicyclic cycloalkyl” includes by definition spirocycloalkyl, bridged, and fused bicycloalkyl groups.

The term “spirocycloalkyl” means a saturated, monovalent bicyclic hydrocarbon group in which the two rings share one common ring carbon atom, and wherein said bicyclic hydrocarbon group contains 5, 6, or 7 carbon atoms, it being possible for said spirocycloalkyl group to be attached to the rest of the molecule via any one of the carbon atoms except the spiro carbon atom. Said spirocycloalkyl group is, for example, spiro[2.2]pentyl, spiro[2.3]hexyl or spiro[2.4]heptyl.

The term “fused bicycloalkyl” means a bicyclic, saturated hydrocarbon ring with 6 or 7 ring atoms in total, in which the two rings share two adjacent ring atoms.

Said fused cycloalkyl group is, for example, a bicyclo[3.1.0]hexanyl or bicyclo[3.2.0]heptanyl group.

The term “bridged bicycloalkyl” means a bicyclic, saturated hydrocarbon ring with 6 or 7 ring atoms in total, in which the two rings share two common ring atoms which are not adjacent. Said bridged cycloalkyl group is, for example, bicyclo[2.1.1]hexanyl or bicyclo[2.2.1]heptanyl group.

The term “—(C₁-C₃-alkyl)-(C₃-C₇-cycloalkyl)” is to be understood as a C₃-C₇-cycloalkyl group as defined above which is attached through any carbon atom of said C₃-C₇-cycloalkyl group to any atom of the C₁-C₃-alkyl group as defined above. The hyphen at the beginning of the group indicates the point of attachment of said (C₁-C₃-alkyl)-(C₃-C₇-cycloalkyl) group to the rest of the molecule. Said (C₁-C₃-alkyl)-(C₃-C₇-cycloalkyl) groups are, for example, cyclopropylmethyl, cyclobutylmethyl, cyclopentylmethyl, cyclohexylmethyl, 2-cyclopropylethyl, 1-cyclopropylethyl, 2-cyclobutylethyl, 1-cyclobutylethyl, 2-cyclopentylethyl, 1-cyclopentylethyl, 2-cyclobutylpropyl, or 1-cyclobutylpropyl.

The term “—OC₃-C₇-cycloalkyl” means a saturated, monovalent, monocyclic group, which contains 3, 4, 5, 6 or 7 carbon atoms, in which the term “C₃-C₇-cycloalkyl” is defined supra, e.g. a cyclopropyloxy, cyclobutyloxy, cyclopentyloxy, cyclohexyloxy, or cycloheptyloxy group.

The term “heterocycloalkyl” is to be understood as meaning a saturated, monovalent, monocyclic or bicyclic hydrocarbon ring with the number of ring atoms as specified in which one or two ring atoms of the hydrocarbon ring is/are replaced by one or two heteroatoms or heteroatom-containing groups independently selected from NH, —NR⁴, N, O, S, SO and SO₂, wherein R⁴ represents C₁-C₅-alkyl optionally substituted with 1 to 5 fluorine atoms. Said heterocycloalkyl can be connected to the rest of the molecule through a carbon or a nitrogen atom, if said nitrogen atom is present.

4- to 7-membered heterocycloalkyl in the context of the invention means a monocyclic or bicyclic, saturated heterocycle with 4, 5, 6 or 7 ring atoms in total, which contains one or two identical or different ring heteroatoms or heteroatom-containing groups from the series NH, —NR⁴, N, O, S, SO and SO₂, wherein R⁴ represents C₁-C₅-alkyl optionally substituted with 1 to 5 fluorine atoms. Said 4- to 7-membered heterocycloalkyl can be bound via a ring carbon or nitrogen atom to the rest of the molecule.

Examples for monocyclic heterocycloalkyl groups are azetidinyl, oxetanyl, thietanyl, pyrrolidinyl, pyrazolidinyl, imidazolidinyl, tetrahydrofuranyl, thiolanyl, 1,1-dioxidothiolanyl, 1,2-oxazolidinyl, 1,3-oxazolidinyl, 1,3-thiazolidinyl, piperidinyl, piperazinyl, tetrahydropyranyl, tetrahydrothiopyranyl, 1,3-dioxanyl, 1,4-dioxanyl, 1,2-oxazinanyl, morpholinyl, thiomorpholinyl, 1,1-dioxidothiomorpholinyl, azepanyl, 1,4-diazepanyl, and 1,4-oxazepanyl.

Particularly, without being limited thereto, said heterocycloalkyl can be a 4-membered ring, such as an azetidinyl, oxetanyl or thietanyl, or a 5-membered ring, such as tetrahydrofuranyl, dioxolinyl, thiolanyl, pyrrolidinyl, imidazolidinyl, pyrazolidinyl, 1,1-dioxidothiolanyl, 1,2-oxazolidinyl, 1,3-oxazolidinyl or 1,3-thiazolidinyl, or a 6-membered ring such as tetrahydropyranyl, tetrahydrothiopyranyl, piperidinyl, morpholinyl, dithianyl, thiomorpholinyl, piperazinyl, 1,3-dioxanyl, 1,4-dioxanyl or 1,2-oxazinanyl, or a 7-membered ring, such as a azepanyl, 1,4-diazepanyl, or 1,4-oxazepanyl, for example.

The term “bicyclic heterocycloalkyl” includes by definition heterospirocycloalkyl, fused and bridged heterobicycloalkyl groups.

The term “heterospirocycloalkyl” means a bicyclic, saturated heterocycle with 6 or 7 ring atoms in total, in which the two rings share one common ring carbon atom, wherein the “heterospirocycloalkyl” contains one or two identical or different ring heteroatoms or heteroatom-containing groups from the series: NH, —NR⁴, N, O, S, SO and SO₂, wherein R⁴ represents C₁-C₅-alkyl optionally substituted with 1 to 5 fluorine atoms; it being possible for said heterospirocycloalkyl group to be attached to the rest of the molecule via any one of the carbon atoms, except the spiro carbon atom, or, if present, a nitrogen atom.

Said heterospirocycloalkyl group is, for example, azaspiro[2.3]hexyl, azaspiro[2.4]-heptanyl, azaspiro[3.3]heptyl, oxazaspiro[3.3]heptyl, thiazaspiro[3.3]heptyl, oxaspiro[3.3]heptyl, diazaspiro[3.3]heptyl or thiazaspiro[3.3]heptyl, or one of the further homologous scaffolds such as spiro[2.3]-, spiro[2.4]-, spiro[3.3]-.

The term “fused heterocycloalkyl” means a bicyclic, saturated heterocycle with 6 or 7 ring atoms in total, in which the two rings share two adjacent ring atoms, which “fused heterocycloalkyl” contains one or two identical or different ring heteroatoms or heteroatom-containing groups from the series: NH, —NR⁴, N, O, S, SO and SO₂, wherein R⁴ represents C₁-C₅-alkyl optionally substituted with 1 to 5 fluorine atoms; it being possible for said fused heterocycloalkyl group to be attached to the rest of the molecule via any one of the carbon atoms or, if present, a nitrogen atom.

Said fused heterocycloalkyl group is, for example, 3-azabicyclo[3.1.0]hexanyl or 3-azabicyclo[3.2.0]heptanyl.

The term “bridged heterocycloalkyl” means a bicyclic, saturated heterocycle with 6 or 7 ring atoms in total, in which the two rings share two common ring atoms which are not adjacent, which “bridged heterocycloalkyl” contains one or two identical or different ring heteroatoms or heteroatom-containing groups from the series: NH, —NR⁴, N, O, S, SO and SO₂, wherein R⁴ represents C₁-C₅-alkyl optionally substituted with 1 to 5 fluorine atoms; it being possible for said bridged heterocycloalkyl group to be attached to the rest of the molecule via any one of the carbon atoms, except the bridgehead carbon atoms, or, if present, a nitrogen atom.

Said bridged heterocycloalkyl group is, for example, azabicyclo[2.2.1]heptyl, oxazabicyclo[2.2.1]heptyl, thiazabicyclo[2.2.1]heptyl, or diazabicyclo[2.2.1]heptyl.

The term “5- to 7-membered lactam” means cyclic amides of amino carboxylic acids, having a 1-azacycloalkan-2-one structure, or analogues having unsaturation or heteroatoms replacing one or more carbon atoms of the ring having a ring size of 5, 6 or 7 ring system atoms. In particular said “5- to 7-membered lactam” means a γ-lactam (gamma-lactam), a δ-lactam (delta-lactam), and an ε-lactam (epsilon-lactam).

The term “heteroaryl” is understood as meaning a monovalent, monocyclic or bicyclic hydrocarbon ring system with at least one aromatic ring, and wherein at least one ring atom of the monovalent, monocyclic or bicyclic hydrocarbon ring system can be replaced by at least one heteroatom or heteroatom-containing group, like NH, N, O, S, SO, and SO₂. The number of ring system atoms is as specified, e.g. a 5- or 6-membered heteroaryl.

“5- or 6-membered heteroaryl” is understood as meaning a monovalent, monocyclic heteroaryl having 5 or 6 ring atoms and wherein one, two or three ring atoms of a monovalent 5-membered hydrocarbon ring system is/are replaced by one, two or three heteroatoms or heteroatom-containing groups independently selected from S, N, NH and O; and wherein one or two ring atoms of a monovalent 6-membered hydrocarbon ring system is/are replaced by one or two nitrogen atoms.

The said 5-membered heteroaryl can be connected through a carbon or a nitrogen atom, if said nitrogen atom is present.

Said 5- or 6-membered heteroaryl group can be a 5-membered heteroaryl group, such as, for example, thienyl, furanyl, pyrrolyl, oxazolyl, thiazolyl, imidazolyl, pyrazolyl, isoxazolyl, isothiazolyl, oxadiazolyl, triazolyl or thiadiazolyl; or a 6-membered heteroaryl group, such as, for example, pyridinyl, pyridazinyl, pyrimidinyl or pyrazinyl.

In general, and unless otherwise mentioned, the term “heteroaryl” includes all possible isomeric forms thereof, e.g. tautomers and positional isomers with respect to the point of linkage to the rest of the molecule. Thus, to give some illustrative non-restricting examples, the term pyridinyl includes pyridin-2-yl, pyridin-3-yl, and pyridin-4-yl; or the term pyrimidinyl includes pyrimidin-2-yl, pyrimidin-4-yl and pyrimidin-5-yl; or the term pyrazolyl includes 1H-pyrazolyl; or the term imidazolyl includes 1H-imidazolyl and 4H-imidazolyl; the term thiophenyl includes 2-thiophenyl and 3-thiophenyl; or the term thiazolyl includes 1,3-thiazol-5-yl, 1,3-thiazol-4-yl and 1,3-thiazol-2-yl.

“Bicyclic 8- to 10-membered heteroaryl” is understood as meaning a bicyclic, monovalent, fused heteroaryl having 8, 9 or 10 ring atoms with at least one aromatic ring and wherein one, two or three ring atoms of a monovalent, 8- to 10-membered bicyclic hydrocarbon ring system is/are replaced by one, two or three heteroatoms or heteroatom-containing groups independently selected from NH, N, O, S, SO and SO₂.

The said bicyclic 8- to 10-membered heteroaryl can be connected through a carbon or a nitrogen atom, if said nitrogen atom is present.

The term “bicyclic 8- to 10-membered heteroaryl” includes by definition fused and bridged heterobicycloalkyl groups.

Particularly, bicyclic heteroaryl is selected from for example, benzofuranyl, benzothienyl, benzothiazolyl, thienopyridinyl, thienopyrimidinyl, benzoxazolyl, benzisoxazolyl, benzimidazolyl, benzotriazolyl, benzothiadiazolyl, indazolyl, indolyl, isoindolyl, etc. or for example, quinolinyl, quinazolinyl, isoquinolinyl, etc.; indolizinyl, or cinnolinyl, phthalazinyl, quinazolinyl, quinoxalinyl, etc.

The term “C₁-C₃” as used throughout this text is to be understood as meaning a group having a finite number of carbon atoms of 1 to 3, i.e. 1, 2, or 3 carbon atoms, e.g. in the context of the definition of “C₁-C₃-alkyl”, it is to be understood as meaning an alkyl group having a finite number of carbon atoms of 1 to 3, i.e. 1, 2, or 3 carbon atoms. It is to be understood further that said term “C₁-C₃” is to be interpreted as any sub-range comprised therein, e.g. C₁-C₂, or C₂-C₃.

The term “C₁-C₅” as used throughout this text is to be understood as meaning a group having a finite number of carbon atoms of 1 to 5, i.e. 1, 2, 3, 4, or 5 carbon atoms, e.g. in the context of the definition of “C₁-C₅-alkyl”, it is to be understood as meaning an alkyl group having a finite number of carbon atoms of 1 to 5, i.e. 1, 2, 3, 4, or 5 carbon atoms. It is to be understood further that said term “C₁-C₅” is to be interpreted as any sub-range comprised therein, e.g. C₁-C₅, C₂-C₅, C₃-C₄, C₂-C₃, C₂-C₄, or C₁-C₄.

The term “C₁-C₃” as used in the context of the definition “—OC₁-C₃-alkyl” is to be understood as meaning an alkyl group, having a finite number of carbon atoms of 1 to 3, i.e. 1, 2 or 3 carbon atoms.

Similarly, the mentioned above applies to “C₁-C₄-alkyl”, “C₁-C₃-alkyl”, “C₁-C₃-alkoxy”, “C₁-C₂-alkyl” or “C₁-C₂-alkoxy”.

Further, as used herein, the term “C₃-C₇”, as used throughout this text, is to be understood as meaning a group having a finite number of carbon atoms of 3 to 7, i.e. 3, 4, 5, 6 or 7 carbon atoms, e.g. in the context of the definition of “C₃-C₇-cycloalkyl”, it is to be understood as meaning a cycloalkyl group having a finite number of carbon atoms of 3 to 7, i.e. 3, 4, 5, 6 or 7 carbon atoms. It is to be understood further that said term “C₃-C₇” is to be interpreted as any sub-range comprised therein, e.g. C₃-C₆, C₄-C₅, C₃-C₅, C₃-C₄, C₄-C₆, or C₅-C₇; particularly C₃-C₆.

Furthermore, as used herein, the term “C₃-C₅”, as used in the present text, e.g. in the context of the definition of “C₃-C₅-cycloalkyl”, means a cycloalkyl group having a finite number of carbon atoms of 3 to 5, i.e. 3, 4 or 5 carbon atoms.

When a range of values is given, said range encompasses each value and sub-range within said range.

For example:

“C₁-C₆” encompasses Cl, C₂, C₃, C₄, C₅, C₆, C₁-C₆, C₁-C₅, C₁-C₄, C₁-C₃, C₁-C₂, C₂-C₆, C₂-C₅, C₂-C₄, C₂-C₃, C₃-C₆, C₃-C₅, C₃-C₄, C₄-C₆, C₄-C₅, and C₅-C₆;

“C₂-C₆” encompasses C₂, C₃, C₄, C₅, C₆, C₂-C₆, C₂-C₅, C₂-C₄, C₂-C₃, C₃-C₆, C₃-C₅, C₃-C₄, C₄-C₆, C₄-C₅, and C₅-C₆;

“C₃-C₁₀” encompasses C₃, C₄, C₅, C₆, C₇, C₈, C₉, C₁₀, C₃-C₁₀, C₃-C₉, C₃-C₈, C₃-C₇, C₃-C₆, C₃-C₅, C₃-C₄, C₄-C₁₀, C₄-C₉, C₄-C₈, C₄-C₇, C₄-C₆, C₄-C₅, C₅-C₁₀, C₅-C₉, C₅-C₈, C₅-C₇, C₅-C₆, C₆-C₁₀, C₆-C₉, C₆-C₈, C₆-C₇, C₇-C₁₀, C₇-C₉, C₇-C₈, C₈-C₁₀, C₈-C₉ and C₉-C₁₀;

“C₃-C₈” encompasses C₃, C₄, C₅, C₆, C₇, C₈, C₃-C₈, C₃-C₇, C₃-C₆, C₃-C₅, C₃-C₄, C₄- C₈, C₄-C₇, C₄-C₆, C₄-C₅, C₅-C₈, C₅-C₇, C₅-C₆, C₆-C₈, C₆-C₇ and C₇-C₈;

“C₃-C₆” encompasses C₃, C₄, C₅, C₆, C₃-C₆, C₃-C₅, C₃-C₄, C₄-C₆, C₄-C₅, and C₅-C₆;

“C₄-C₈” encompasses C₄, C₅, C₆, C₇, C₈, C₄-C₈, C₄-C₇, C₄-C₆, C₄-C₅, C₅-C₈, C₅-C₇, C₅-C₆, C₆-C₈, C₆-C₇ and C₇-C₈;

“C₄-C₇” encompasses C₄, C₅, C₆, C₇, C₄-C₇, C₄-C₆, C₄-C₅, C₅-C₇, C₅-C₆ and C₆-C₇;

“C₄-C₆” encompasses C₄, C₅, C₆, C₄-C₆, C₄-C₅ and C₅-C₆;

“C₅-C₁₀” encompasses C₅, C₆, C₇, C₈, C₉, C₁₀, C₅-C₁₀, C₅-C₉, C₅-C₈, C₅-C₇, C₅-C₆, C₆- C₁₀, C₆-C₉, C₆-C₈, C₆-C₇, C₇-C₁₀, C₇-C₉, C₇-C₈, C₈-C₁₀, C₈-C₉ and C₉-C₁₀;

“C₆-C₁₀” encompasses C₆, C₇, C₈, C₉, C₁₀, C₆-C₁₀, C₆-C₉, C₆-C₈, C₆-C₇, C₇-C₁₀, C₇-C₉, C₇-C₈, C₈-C₁₀, C₈-C₉ and C₉-C₁₀.

As used herein, the term “leaving group” means an atom or a group of atoms that is displaced in a chemical reaction as stable species taking with it the bonding electrons. In particular, such a leaving group is selected from the group comprising: halide, in particular fluoride, chloride, bromide or iodide, (methylsulfonyl)oxy, [(trifluoromethyl)sulfonyl]oxy, [(nonafluorobutyl)-sulfonyl]oxy, (phenylsulfonyl)oxy, [(4-methylphenyl)sulfonyl]oxy, [(4-bromo-phenyl)sulfonyl]oxy, [(4-nitrophenyl)sulfonyl]oxy, [(2-nitrophenyl)sulfonyl]oxy, [(4-isopropylphenyl)sulfonyl]oxy, [(2,4,6-triisopropylphenyl)sulfonyl]oxy, [(2,4,6-trimethyl-phenyl)sulfonyl]oxy, [(4-tert-butylphenyl)sulfonyl]oxy and [(4-methoxyphenyl)sulfonyl]oxy.

It is possible for the compounds of general formula (I) to exist as isotopic variants. The invention therefore includes one or more isotopic variant(s) of the compounds of general formula (I), particularly deuterium-containing compounds of general formula (I).

The term “Isotopic variant” of a compound or a reagent is defined as a compound exhibiting an unnatural proportion of one or more of the isotopes that constitute such a compound.

The term “Isotopic variant of the compound of general formula (I)” is defined as a compound of general formula (I) exhibiting an unnatural proportion of one or more of the isotopes that constitute such a compound.

The expression “unnatural proportion” means a proportion of such isotope, which is higher than its natural abundance. The natural abundances of isotopes to be applied in this context are described in “Isotopic Compositions of the Elements 1997”, Pure Appl. Chem., 70(1), 217-235, 1998, which is incorporated herein by reference.

Examples of such isotopes include stable and radioactive isotopes of hydrogen, carbon, nitrogen, oxygen, phosphorus, sulfur, fluorine, chlorine, bromine and iodine, such as ²H (deuterium), ³H (tritium), ¹¹C, ¹³C, ¹⁴C, ¹⁵N, ¹⁷O, ¹⁸O, ³²P, ³³P, ³³S, ³⁴S, ³⁵S, ³⁶S, ¹⁸F, ³⁶Cl, ⁸²Br, ¹²³I, ¹²⁴I, ¹²⁵I, ¹²⁹I and ¹³¹I, respectively.

With respect to the treatment and/or prophylaxis of the disorders specified herein the isotopic variant(s) of the compounds of general formula (I) preferably contain deuterium (“deuterium-containing compounds of general formula (I)”). Isotopic variants of the compounds of general formula (I) in which one or more radioactive isotopes, such as ³H or ¹⁴C, are incorporated are useful e.g. in drug and/or substrate tissue distribution studies. These isotopes are particularly preferred for the ease of their incorporation and detectability. Positron emitting isotopes such as ¹⁸F or ¹¹C may be incorporated into a compound of general formula (I). These isotopic variants of the compounds of general formula (I) are useful for in vivo imaging applications. Deuterium-containing and ¹³C-containing compounds of general formula (I) can be used in mass spectrometry analyses in the context of preclinical or clinical studies.

Isotopic variants of the compounds of general formula (I) can generally be prepared by methods known to a person skilled in the art, such as those described in the schemes and/or examples herein, by substituting a reagent for an isotopic variant of said reagent, preferably for a deuterium-containing reagent. Depending on the desired sites of deuteration, in some cases deuterium from D₂O can be incorporated either directly into the compounds or into reagents that are useful for synthesizing such compounds. Deuterium gas is also a useful reagent for incorporating deuterium into molecules. Catalytic deuteration of olefinic bonds and acetylenic bonds is a rapid route for incorporation of deuterium. Metal catalysts (i.e. Pd, Pt, and Rh) in the presence of deuterium gas can be used to directly exchange deuterium for hydrogen in functional groups containing hydrocarbons. A variety of deuterated reagents and synthetic building blocks are commercially available from companies such as for example C/D/N Isotopes, Quebec, Canada; Cambridge Isotope Laboratories Inc., Andover, Mass., USA; and CombiPhos Catalysts, Inc., Princeton, N.J., USA.

The term “deuterium-containing compound of general formula (I)” is defined as a compound of general formula (I), in which one or more hydrogen atom(s) is/are replaced by one or more deuterium atom(s) and in which the abundance of deuterium at each deuterated position of the compound of general formula (I) is higher than the natural abundance of deuterium, which is about 0.015%. Particularly, in a deuterium-containing compound of general formula (I) the abundance of deuterium at each deuterated position of the compound of general formula (I) is higher than 10%, 20%, 30%, 40%, 50%, 60%, 70% or 80%, preferably higher than 90%, 95%, 96% or 97%, even more preferably higher than 98% or 99% at said position(s). It is understood that the abundance of deuterium at each deuterated position is independent of the abundance of deuterium at other deuterated position(s).

The selective incorporation of one or more deuterium atom(s) into a compound of general formula (I) may alter the physicochemical properties (such as for example acidity [C. L. Perrin, et al., J. Am. Chem. Soc., 2007, 129, 4490], basicity [C. L. Perrin et al., J. Am. Chem. Soc., 2005, 127, 9641], lipophilicity [B. Testa et al., Int. J. Pharm., 1984, 19(3), 271] and/or the metabolic profile of the molecule and may result in changes in the ratio of parent compound to metabolites or in the amounts of metabolites formed. Such changes may result in certain therapeutic advantages and hence may be preferred in some circumstances. Reduced rates of metabolism and metabolic switching, where the ratio of metabolites is changed, have been reported (A. E. Mutlib et al., Toxicol. Appl. Pharmacol., 2000, 169, 102). These changes in the exposure to parent drug and metabolites can have important consequences with respect to the pharmacodynamics, tolerability and efficacy of a deuterium-containing compound of general formula (I). In some cases, deuterium substitution reduces or eliminates the formation of an undesired or toxic metabolite and enhances the formation of a desired metabolite (e.g. Nevirapine: A. M. Sharma et al., Chem. Res. Toxicol., 2013, 26, 410; Efavirenz: A. E. Mutlib et al., Toxicol. Appl. Pharmacol., 2000, 169, 102; both incorporated herein by reference). In other cases, the major effect of deuteration is to reduce the rate of systemic clearance. As a result, the biological half-life of the compound is increased. The potential clinical benefits would include the ability to maintain similar systemic exposure with decreased peak levels and increased trough levels. This could result in lower side effects and enhanced efficacy, depending on the particular compound's pharmacokinetic/pharmacodynamic relationship. ML-337 (C. J. Wenthur et al., J. Med. Chem., 2013, 56, 5208; incorporated herein by reference) and Odanacatib (K. Kassahun et al., WO2012/112363; incorporated herein by reference) are examples for this deuterium effect. Still other cases have been reported in which reduced rates of metabolism result in an increase in exposure of the drug without changing the rate of systemic clearance (e.g. Rofecoxib: F. Schneider et al., Arzneim. Forsch./Drug. Res., 2006, 56, 295; Telaprevir: F. Maltais et al., J. Med. Chem., 2009, 52, 7993; incorporated herein by reference). Deuterated drugs showing this effect may have reduced dosing requirements (e.g. lower number of doses or lower dosage to achieve the desired effect) and/or may produce lower metabolite loads. A compound of general formula (I) may have multiple potential sites of attack for metabolism. To optimize the above-described effects on physicochemical properties and metabolic profile, deuterium-containing compounds of general formula (I) having a certain pattern of one or more deuterium-hydrogen exchange(s) can be selected. Particularly, the deuterium atom(s) of deuterium-containing compound(s) of general formula (I) is/are attached to a carbon atom and/or is/are located at those positions of the compound of general formula (I), which are sites of attack for metabolizing enzymes such as e.g. cytochrome P₄₅₀.

Optical isomers can be obtained by resolution of the racemic mixtures according to conventional processes, for example, by the formation of diastereoisomeric salts using an optically active acid or base or formation of covalent diastereomers. Examples of appropriate acids are tartaric, diacetyltartaric, ditoluoyltartaric and camphorsulfonic acid. Mixtures of diastereoisomers can be separated into their individual diastereomers on the basis of their physical and/or chemical differences by methods known in the art, for example, by chromatography or fractional crystallisation. The optically active bases or acids are then liberated from the separated diastereomeric salts. A different process for separation of optical isomers involves the use of chiral chromatography (e.g., chiral HPLC columns), with or without conventional derivatisation, optimally chosen to maximise the separation of the enantiomers. Suitable chiral HPLC columns are manufactured by Daicel, e.g., Chiracel OD and Chiracel OJ among many others, all routinely selectable. Enzymatic separations, with or without derivatisation, are also useful. The optically active compounds of this invention can likewise be obtained by chiral syntheses utilizing optically active starting materials.

In order to limit different types of isomers from each other reference is made to IUPAC Rules Section E (Pure Appl Chem 45, 11-30, 1976), thereby incorporated herein.

Further, the compounds of the present invention may exist as tautomers.

The present invention includes all possible tautomers of the compounds of the present invention as single tautomers, or as any mixture of said tautomers, in any ratio.

The present invention includes all possible stereoisomers of the compounds of the present invention as single stereoisomers, or as any mixture of said stereoisomers, e.g. (R)- or (S)-isomers, in any ratio. Isolation of a single stereoisomer, e.g. a single enantiomer or a single diastereomer, of a compound of the present invention is achieved by any suitable state of the art method, such as chromatography, especially chiral chromatography, for example. The 1,2-cyclopropylamides of the invention have to be understood, unless stated otherwise, as relating to both cis and trans isomers referred to R⁷ and R⁸, as either single entantiomers or a mixture of enantiomers. Preferred are mixtures of trans enantionmers, if not stated otherwise.

The present invention also relates to useful forms of the compounds as disclosed herein, such as hydrates, solvates, and salts, in particular pharmaceutically acceptable salts.

Where the plural form of the word compounds, salts, polymorphs, hydrates, solvates and the like, is used herein, this is taken to mean also a single compound, salt, polymorph, isomer, hydrate, solvate or the like.

By “stable compound” or “stable structure” is meant a compound that is sufficiently robust to survive isolation to a useful degree of purity from a reaction mixture, and formulation into an efficacious therapeutic agent.

The compounds of the present invention can exist as a hydrate, or as a solvate, wherein the compounds of the present invention contain polar solvents, in particular water, methanol or ethanol for example as structural element of the crystal lattice of the compounds. The amount of polar solvents, in particular water, may exist in a stoichiometric or non-stoichiometric ratio. In the case of stoichiometric solvates, e.g. a hydrate, hemi-, (semi-), mono-, sesqui-, di-, tri-, tetra-, penta- etc. solvates or hydrates, respectively, are possible. The present invention includes all such hydrates or solvates.

Further, the compounds of the present invention can exist in free form, e.g. as a free base, or as a free acid, or as a zwitterion, or can exist in the form of a salt. Said salt may be any salt, either an organic or inorganic addition salt, particularly any pharmaceutically acceptable organic or inorganic addition salt, customarily used in pharmacy.

The term “pharmaceutically acceptable salt” refers to a relatively non-toxic, inorganic or organic acid addition salt of a compound of the present invention. For example, see S. M. Berge, et al. “Pharmaceutical Salts,” J. Pharm. Sci. 1977, 66, 1-19, incorporated herein by reference. A suitable pharmaceutically acceptable salt of the compounds of the present invention may be, for example, an acid-addition salt of a compound of the present invention bearing a nitrogen atom, in a chain or in a ring, for example, which is sufficiently basic, such as an acid-addition salt with an inorganic acid, such as hydrochloric, hydrobromic, hydroiodic, sulfuric, bisulfuric, phosphoric, or nitric acid, for example, or with an organic acid, such as formic, acetic, acetoacetic, pyruvic, trifluoroacetic, propionic, butyric, hexanoic, heptanoic, undecanoic, lauric, benzoic, salicylic, 2-(4-hydroxybenzoyl)-benzoic, camphoric, cinnamic, cyclopentanepropionic, digluconic, 3-hydroxy-2-naphthoic, nicotinic, pamoic, pectinic, persulfuric, 3-phenylpropionic, picric, pivalic, 2-hydroxyethanesulfonate, itaconic, sulfamic, trifluoromethanesulfonic, dodecylsulfuric, ethansulfonic, benzenesulfonic, para-toluenesulfonic, methansulfonic, 2-naphthalenesulfonic, naphthalinedisulfonic, camphorsulfonic acid, citric, tartaric, stearic, lactic, oxalic, malonic, succinic, malic, adipic, alginic, maleic, fumaric, D-gluconic, mandelic, ascorbic, glucoheptanoic, glycerophosphoric, aspartic, sulfosalicylic, hemisulfuric, or thiocyanic acid, for example.

Further, another pharmaceutically acceptable salt of a compound of the present invention which is sufficiently acidic, is an alkali metal salt, for example a sodium or potassium salt, an alkaline earth metal salt, for example a calcium or magnesium salt, an ammonium salt or a salt with an organic base which affords a physiologically acceptable cation, for example a salt with N-methyl-glucamine, dimethyl-glucamine, ethyl-glucamine, lysine, dicyclohexylamine, 1,6-hexadiamine, ethanolamine, glucosamine, sarcosine, serinol, tris-hydroxy-methyl-aminomethane, aminopropandiol, sovak-base, 1-amino-2,3,4-butantriol.

Additionally, basic nitrogen containing groups may be quaternised with such agents as lower alkyl halides such as methyl, ethyl, propyl, and butyl chlorides, bromides and iodides; dialkyl sulfates like dimethyl, diethyl, and dibutyl sulfate; and diamyl sulfates, long chain halides such as decyl, lauryl, myristyl and stearyl chlorides, bromides and iodides, aralkyl halides like benzyl and phenethyl bromides and others.

Those skilled in the art will further recognise that acid addition salts of the claimed compounds may be prepared by reaction of the compounds with the appropriate inorganic or organic acid via any of a number of known methods. Alternatively, alkali and alkaline earth metal salts of acidic compounds of the invention are prepared by reacting the compounds of the invention with the appropriate base via a variety of known methods.

The present invention includes all possible salts of the compounds of the present invention as single salts, or as any mixture of said salts, in any ratio.

Unless otherwise indicated, the compounds of the present invention are also referred to isomers, enantiomers, diastereomers, racemates, hydrates, solvates, a salt thereof, or a mixture of same.

As used herein, the term “in vivo hydrolysable ester” is understood as meaning an in vivo hydrolysable ester of a compound of the present invention containing a carboxy or hydroxy group, for example, a pharmaceutically acceptable ester that is hydrolysed in the human or animal body to produce the parent acid or alcohol. Suitable pharmaceutically acceptable esters for carboxy include for example alkyl, cycloalkyl and optionally substituted phenylalkyl, in particular benzyl esters, C₁-C₆ alkoxymethyl esters, e.g. methoxymethyl, C₁-C₆ alkanoyloxymethyl esters, e.g. pivaloyloxymethyl, phthalidyl esters, C₃-C₈ cycloalkoxy-carbonyloxy-C₁-C₆ alkyl esters, e.g. 1-cyclohexylcarbonyloxyethyl; 1,3-dioxolen-2-onylmethyl esters, e.g. 5-methyl-1,3-dioxolen-2-onylmethyl; and C₁-C₆-alkoxycarbonyloxyethyl esters, e.g. 1-methoxycarbonyloxyethyl, and may be formed at any carboxy group in the compounds of this invention. An in vivo hydrolysable ester of a compound of the present invention containing a hydroxy group includes inorganic esters such as phosphate esters and [alpha]-acyloxyalkyl ethers and related compounds which as a result of the in vivo hydrolysis of the ester breakdown to give the parent hydroxy group. Examples of [alpha]-acyloxyalkyl ethers include acetoxymethoxy and 2,2-dimethylpropionyloxymethoxy. A selection of in vivo hydrolysable ester forming groups for hydroxy include alkanoyl, benzoyl, phenylacetyl and substituted benzoyl and phenylacetyl, alkoxycarbonyl (to give alkyl carbonate esters), dialkylcarbamoyl and N-(dialkylaminoethyl)-N-alkylcarbamoyl (to give carbamates), dialkylaminoacetyl and carboxyacetyl. The present invention covers all such esters.

Furthermore, the present invention includes all possible crystalline forms, or polymorphs, of the compounds of the present invention, either as single polymorphs, or as a mixture of more than one polymorph, in any ratio.

The present invention in particular covers carboxylic acid aromatic amides of general formula (I):

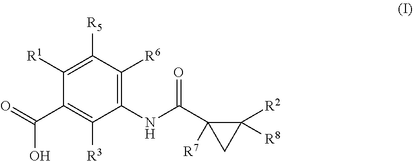

in which
R¹ represents
   5- or 6-membered heteroaryl, wherein said 5-membered heteroaryl contains 1, 2 or 3 heteroatoms selected from the group consisting of S, N, and O, and said 6-membered heteroaryl contains 1 or 2 N,
   wherein said R¹ is optionally substituted at one or more carbon atoms with 1 to 3 substituents R^{1a} which are the same or different wherein R^{1a} represents C₁-C₅-alkyl, C₃-C₅-cycloalkyl, —(C₁-C₃-alkyl)-(C₃-C₅-cycloalkyl), —OC₁-C₅-alkyl, —OC₃-C₅-cycloalkyl, or F and
   if R^{1a} represents C₁-C₅-alkyl, C₃-C₅-cycloalkyl, —(C₁-C₃-alkyl)-(C₃-C₅-cycloalkyl), —OC₁-C₅-alkyl or —OC₃-C₅-cycloalkyl, said C₁-C₅-alkyl, C₃-C₅-cycloalkyl, —(C₁-C₃-alkyl)-(C₃-C₅-cycloalkyl), —OC₁-C₅-alkyl and —OC₃-C₅-cycloalkyl independently are optionally substituted with one or more substituents independently selected from the group consisting of OH and F;
R² represents
   —(CH₂)ₚ-phenyl,
   wherein said R² is optionally substituted at one or more carbon atoms with 1 to 3 substituents R^{2a} which are the same or different and wherein R^{2a} is selected from the group consisting of C₁-C₅-alkyl, —OC₁-C₅-alkyl, halogen, OH and CN, wherein said C₁-C₅-alkyl and —OC₁-C₅-alkyl independently are optionally substituted with (a) substituent(s) independently selected from the group consisting of OH, and 1 to 5 fluorine atoms;
p is 0 or 1;
R³ represents H or fluorine;
R⁴ represents C₁-C₅-alkyl, optionally substituted with 1-5 fluorine atoms;
R⁵ represents H, halogen, CN, OH, C₁-C₅-alkyl, or —OC₁-C₅-alkyl, wherein said C₁-C₅-alkyl and —OC₁-C₅-alkyl are optionally substituted with 1 to 5 fluorine atoms;
R⁶ represents H, halogen, CN, OH, C₁-C₅-alkyl, or —OC₁-C₅-alkyl wherein said C₁-C₅-alkyl and —OC₁-C₅-alkyl are optionally substituted with 1 to 5 fluorine atoms; and
R⁷ and R⁸ independently represent H, or C₁-C₃-alkyl, wherein the C₁-C₃-alkyl is independently optionally substituted with 1 to 3 fluorine atoms;

or an isomer, enantiomer, diastereomer, racemate, hydrate, solvate, or a salt thereof, or a mixture of the same.

In accordance with one aspect, the present invention covers compounds of general formula (I)

wherein
R¹ represents 5- or 6-membered heteroaryl, wherein said 5-membered heteroaryl contains 1, 2 or 3 heteroatoms selected from the group consisting of S, N, and O, and said 6-membered heteroaryl contains 1 or 2 N,
   wherein R¹ is optionally substituted as defined in formula (I).

Also preferred are compounds of general formula (I), wherein
R¹ represents 5- or 6-membered heteroaryl, wherein said 5-membered heteroaryl contains 1, 2 or 3 heteroatoms selected from the group consisting of S, N, and O, and said 6-membered heteroaryl contains 1 or 2 N,
   wherein said R¹ is optionally substituted at one or more carbon atoms with 1 to 3 substituents R^{1a} which are the same or different wherein R^{1a} represents C₁-C₅-alkyl, C₃-C₅-cycloalkyl, —(C₁-C₃-alkyl)-(C₃-C₅-cycloalkyl), —OC₁-C₅-alkyl, —OC₃-C₅-cycloalkyl, or halogen, and
   if R^{1a} represents C₁-C₅-alkyl, C₃-C₅-cycloalkyl, —(C₁-C₃-alkyl)-(C₃-C₅-cycloalkyl), —OC₁-C₅-alkyl or —OC₃-C₅-cycloalkyl, said C₁-C₅-alkyl, C₃-C₅-cycloalkyl, —(C₁-C₃-alkyl)-(C₃-C₅-cycloalkyl), —OC₁-C₅-alkyl and —OC₃-C₅-cycloalkyl independently are optionally substituted with one or more substituents independently selected from the group consisting of OH, OR⁴ and F.

Also preferred are compounds of general formula (I), wherein
R¹ represents 6-membered heteroaryl containing 1 or 2 N, in particular pyridinyl, pyrimidinyl or pyrazinyl,
   wherein said R¹ is optionally substituted at one or more carbon atoms with 1 to 3 substituents R^{1a} which are the same or different wherein R^{1a} represents C₁-C₅-alkyl, C₃-C₅-cycloalkyl, —(C₁-C₃-alkyl)-(C₃-C₅-cycloalkyl), —OC₁-C₅-alkyl, —OC₃-C₅-cycloalkyl, or halogen, and
   if R^{1a} represents C₁-C₅-alkyl, C₃-C₅-cycloalkyl, —(C₁-C₃-alkyl)-(C₃-C₅-cycloalkyl), —OC₁-C₅-alkyl or —OC₃-C₅-cycloalkyl, said C₁-C₅-alkyl, C₃-C₅-cycloalkyl, —(C₁-C₃-alkyl)-(C₃-C₅-cycloalkyl), —OC₁-C₅-alkyl and —OC₃-C₅-cycloalkyl independently are optionally substituted with one or more substituents independently selected from the group consisting of OH, OR⁴ and F.

Also preferred are compounds of general formula (I), wherein
R¹ represents 6-membered heteroaryl containing 2 N, in particular pyridinyl, or pyrimidinyl, wherein said R¹ is optionally substituted at one or more carbon atoms with 1 to 3 substituents R^{1a} which are the same or different wherein R^{1a} represents C₁-C₅-alkyl, C₃-C₅-cycloalkyl, —(C₁-C₃-alkyl)-(C₃-C₅-cycloalkyl), —OC₁-C₅-alkyl, —OC₃-C₅-cycloalkyl, or halogen, and
   if R^{1a} represents C₁-C₅-alkyl, C₃-C₅-cycloalkyl, —(C₁-C₃-alkyl)-(C₃-C₅-cycloalkyl), —OC₁-C₅-alkyl or —OC₃-C₅-cycloalkyl, said C₁-C₅-alkyl, C₃-C₅-cycloalkyl, —(C₁-C₃-alkyl)-(C₃-C₅-cycloalkyl), —OC₁-C₅-alkyl and —OC₃-C₅-cycloalkyl independently are optionally substituted with one or more substituents independently selected from the group consisting of OH, OR⁴ and F.

Also preferred are compounds of general formula (I), wherein
R¹ represents pyridinyl, in particular pyridin-3-yl,
   wherein said R¹ is optionally substituted at one or more carbon atoms with 1 to 3 substituents independently selected from the group consisting of C₁-C₅-alkyl, C₃-C₅-cycloalkyl, —(C₁-C₃-alkyl)-(C₃-C₅-cycloalkyl), —OC₁-C₅-alkyl, —OC₃-C₅-cycloalkyl, or halogen, and
   wherein one of said substituents is preferably positioned para to the carbon atom which links the pyridinyl, in particular pyridin-3-yl, to the rest of the molecule; and wherein said C₁-C₅-alkyl, C₃-C₅-cycloalkyl, —(C₁-C₃-alkyl)-(C₃-C₅-cycloalkyl), —OC₁-C₅-alkyl and —OC₃-C₅-cycloalkyl independently are optionally substituted with one or more substituents independently selected from the group consisting of OH, OR⁴ and F, in particular F; and wherein
R⁴ has the same meaning as defined above in general formula (I).

Also preferred are compounds of general formula (I), wherein
R¹ represents pyridinyl, in particular pyridin-3-yl, wherein said R¹ is substituted at the carbon atom positioned para to the atom to which the pyridinyl is attached to the rest of the molecule with a C₁-C₅-alkyl, more preferably attached to the carbon atom at the 6-position of pyridin-3-yl, and
wherein said C₁-C₅-alkyl is optionally substituted with one or more substituents independently selected from the group consisting of OH, OR⁴ and F, in particular F; and wherein
R⁴ has the same meaning as defined above in general formula (I).

Also preferred are compounds of general formula (I), wherein
R¹ represents
   pyridinyl, in particular pyridin-3-yl,
   wherein said R¹ is independently substituted at one or more carbon atoms with 1 or 2 C₁-C₅-alkyl substituents, which are the same or different, selected from the group consisting of methyl, ethyl, methoxy, ethoxy, trifluoromethyl, difluoromethyl, 1,1-difluoroethyl, 1,1-difluoropropyl and 2,2,2-trifluoroethyl, and
   wherein one of said substituents is preferably positioned para to the atom to which the pyridinyl is attached to the rest of the molecule, more preferably positioned at the carbon atom at the 6-position of pyridin-3-yl.

Also preferred are compounds of general formula (I), wherein
R¹ represents pyridinyl, in particular pyridin-3-yl,
   wherein said R¹ is substituted with an optionally substituted C₁-C₅-alkyl substituent attached at the carbon atom positioned para to the atom to which the pyridinyl is attached to the rest of the molecule, in particular positioned at the carbon atom at the 6-position of pyridin-3-yl,
   wherein said optionally substituted C₁-C₅-alkyl substituent is selected from the group consisting of methyl, ethyl, methoxy, ethoxy, trifluoromethyl, difluoromethyl, 1,1-difluoroethyl, 1,1-difluoropropyl and 2,2,2-trifluoroethyl.

Also preferred are compounds of general formula (I), wherein
R¹ represents 5-membered heteroaryl wherein said 5-membered heteroaryl contains 1, 2 or 3 heteroatoms or heteroatom-containing groups independently selected from the group consisting of S, N, and O, in particular pyrazolyl, thiazolyl, imidazolyl, or thiophenyl, wherein said R¹ is optionally substituted at one or more carbon atoms with 1 or 2 substituents R^{1a} which are the same or different, wherein R^{1a} represents C₁-C₅-alkyl, C₃-C₇-cycloalkyl, —(C₁-C₃-alkyl)-(C₃-C₇-cycloalkyl), —OC₁-C₅-alkyl, —OC₃-C₇-cycloalkyl, halogen, or CN, and
   wherein independently each ring nitrogen atom, if present, of said R¹ is optionally substituted with a substituent R^{1b}, wherein R^{1b} represents C₁-C₅-alkyl, —(C₁-C₃-alkyl)-(C₃-C₇-cycloalkyl), or C₃-C₇-cycloalkyl, and
   if R^{1a} represents C₁-C₅-alkyl, C₃-C₇-cycloalkyl, —(C₁-C₃-alkyl)-(C₃-C₇-cycloalkyl), —OC₁-C₅-alkyl, or —OC₃-C₇-cycloalkyl, and/or if R^{1b} represents C₁-C₅-alkyl, —(C₁-C₃-alkyl)-(C₃-C₇-cycloalkyl) or C₃-C₇-cycloalkyl, said C₁-C₅-alkyl, C₃-C₇-cycloalkyl, —(C₁-C₃-alkyl)-(C₃-C₇-cycloalkyl), —OC₁-C₅-alkyl, and —OC₃-C₇-cycloalkyl independently are optionally substituted with one or more substituents independently selected from the group consisting of methyl, ethyl, OH, OR⁴ and F; and wherein
R⁴ has the same meaning as defined above in general formula (I).

Also preferred are compounds of general formula (I), wherein
R¹ represents pyrazolyl, in particular pyrazol-4-yl, optionally substituted at one or more carbon atoms with 1 or 2 substituents R^{1a} which are the same or different, wherein R^{1a} represents C₁-C₅-alkyl, C₃-C₇-cycloalkyl, —(C₁-C₃-alkyl)-(C₃-C₇-cycloalkyl), —OC₁-C₅-alkyl, —OC₃-C₇-cycloalkyl, halogen, or CN, and
   wherein independently each ring nitrogen atom of said R¹ is optionally substituted with a substituent R^{1b}, wherein R^{1b} represents C₁-C₅-alkyl, —(C₁-C₃-alkyl)-(C₃-C₇-cycloalkyl), or C₃-C₇-cycloalkyl, and
   if R^{1a} represents C₁-C₅-alkyl, C₃-C₇-cycloalkyl, —(C₁-C₃-alkyl)-(C₃-C₇-cycloalkyl), —OC₁-C₅-alkyl, or —OC₃-C₇-cycloalkyl, and/or if R^{1b} represents C₁-C₅-alkyl, —(C₁-C₃-alkyl)-(C₃-C₇-cycloalkyl), or C₃-C₇-cycloalkyl, said C₁-C₅-alkyl, C₃-C₇-cycloalkyl, —(C₁-C₃-alkyl)-(C₃-C₇-cycloalkyl), —OC₁-C₅-alkyl and —OC₃-C₇-cycloalkyl independently are optionally substituted with one or more substituents independently selected from the group consisting of methyl, ethyl, OH, OR⁴ and F; and wherein
R⁴ has the same meaning as defined above in general formula (I).

Also preferred are compounds of general formula (I), wherein
R¹ represents pyrazol-4-yl substituted at the nitrogen atom at position 1 with a substituent R^{1b}, wherein R^{1b} represents C₁-C₅-alkyl, —(C₁-C₃-alkyl)-(C₃-C₇-cycloalkyl) or C₃-C₇-cycloalkyl, wherein said C₁-C₅-alkyl, C₃-C₇-cycloalkyl and —(C₁-C₃-alkyl)-(C₃-C₇-cycloalkyl) is optionally substituted with one or more substituents independently selected from the group consisting of methyl, ethyl, OH, OR⁴ and F; and wherein
R⁴ has the same meaning as defined above in general formula (I).

Additionally preferred are compounds of general formula (I), wherein
R¹ represents pyrazolyl, in particular pyrazol-4-yl, wherein said R¹ is optionally substituted with 1 or 2 R^{1b} which are the same or different, wherein R^{1b} represents C₁-C₅-alkyl, C₃-C₇-cycloalkyl or —(C₁-C₃-alkyl)-(C₃-C₇-cycloalkyl), and wherein one of said substituents R^{1b} is attached to the pyrazolyl nitrogen atom at position 1, preferably attached to the pyrazol-4-yl nitrogen atom at position 1, and wherein said C₁-C₅-alkyl, C₃-C₇-cycloalkyl and —(C₁-C₃-alkyl)-(C₃-C₇-cycloalkyl) independently are optionally substituted with one or more substituents independently selected from the group consisting of methyl, ethyl, OH, OR⁴, and F; and wherein
R⁴ has the same meaning as defined above in general formula (I).

Additionally preferred are compounds of general formula (I), wherein
R¹ represents pyrazol-4-yl substituted at the nitrogen atom at position 1 with C₃-C₇-cycloalkyl, wherein said C₃-C₇-cycloalkyl is optionally substituted with one or more substituents independently selected from the group consisting of methyl, ethyl, OH, OR⁴, and F; and wherein
R⁴ has the same meaning as defined above in general formula (I).

Additionally preferred are compounds of general formula (I), wherein
R¹ represents pyrazol-4-yl substituted at the nitrogen atom at position 1 with C₃-C₇-cycloalkyl, wherein said C₃-C₇-cycloalkyl is optionally substituted with one or more substituents independently selected from the group consisting of methyl, OH, OR⁴, and F; and wherein
R⁴ has the same meaning as defined above in general formula (I).

Also preferred are compounds of general formula (I), wherein
R¹ represents pyrazol-4-yl substituted at the nitrogen atom at position 1 with a substituent selected from the group consisting of methyl, ethyl, propyl, propan-2-yl, 2-methylpropyl, tertbutyl, butan-2-yl, cyclobutyl, 2,2-dimethylpropyl, 3-methylbutan-2-yl, cyclopentyl, cyclohexyl, 1-cyclopropylmethyl, 1-cyclopropylethyl, 1-cyclobutylmethyl, 1-(1-methylcyclopropyl)methyl and 2,2,2-trifluoroethyl, in particular ethyl, propan-2-yl, 2-methylpropyl, butan-2-yl, cyclobutyl, cyclopentyl, 2,2-dimethylpropyl, 1-cyclopropylmethyl, 1-cyclopropylethyl, 1-(1-methylcyclopropyl)methyl, and 1-cyclobutylmethyl.

Particularly preferred are compounds of general formula (I), wherein
R¹ represents pyrazol-4-yl, substituted at the nitrogen atom at position 1 with cyclobutyl.

Additionally preferred are compounds of general formula (I), wherein
R¹ represents pyridinyl, or pyrazolyl,
   wherein said R¹ is optionally substituted at one or more carbon atoms with 1 or 2 substituents R^{1a} which are the same or different selected from the group consisting of C₁-C₅-alkyl, C₃-C₇-cycloalkyl, —(C₁-C₃-alkyl)-(C₃-C₇-cycloalkyl), —OC₁-C₅-alkyl, —OC₃-C₇-cycloalkyl, halogen and CN, and
   wherein independently said R¹ is optionally substituted at a nitrogen atom with 1 substituent R^{1b} selected from the group consisting of C₁-C₅-alkyl, —(C₁-C₃-alkyl)-(C₃-C₇-cycloalkyl) and C₃-C₇-cycloalkyl,
      wherein said C₁-C₅-alkyl, C₃-C₇-cycloalkyl, —(C₁-C₃-alkyl)-(C₃-C₇-cycloalkyl), —OC₁-C₅-alkyl and —OC₃-C₇-cycloalkyl are optionally substituted with one or more substituents independently selected from the group consisting of OH, OR⁴ and fluorine; and wherein
R⁴ has the same meaning as defined above in general formula (I).

Additionally preferred are compounds of general formula (I), wherein
R¹ represents pyridinyl, or pyrazolyl,
   wherein said R¹ is optionally substituted at one carbon atom with a C₁-C₅-alkyl, and wherein independently said R¹ is optionally substituted at a nitrogen atom with a C₃-C₇-cycloalkyl,
      wherein said C₁-C₅-alkyl, and C₃-C₇-cycloalkyl are independently optionally substituted with one or more substituents independently selected from the group consisting of OH, OR⁴ and F; and wherein
      R⁴ has the same meaning as defined above in general formula (I).

Additionally preferred are compounds of general formula (I), wherein
R¹ represents pyridinyl, or pyrazolyl,
   wherein said R¹ is optionally substituted at one carbon atom with an unsubstituted C₁-C₅-alkyl, in particular tert-butyl, and
   wherein independently said R¹ is optionally substituted at a nitrogen atom with an unsubstituted C₃-C₇-cycloalkyl, in particular cyclobutyl.

In accordance with a further aspect, the present invention covers compounds of general formula (I), wherein
R⁴ represents C₁-C₅-alkyl, in particular methyl, ethyl, propyl, or butyl optionally substituted with 1 to 3 fluorine atoms.
   Also preferred are compounds of general formula (I), wherein
R⁴ represents methyl, difluoromethyl, or trifluoromethyl.

In accordance with a further aspect, the present invention covers compounds of general formula (I), wherein
R³ represents H.

In accordance with a further aspect, the present invention covers compounds of general formula (I), wherein
R⁵ represents H, F, Cl, or methyl, in particular H or F.

Particularly preferred are compounds of general formula (I), wherein
R⁵ represents H or F, in particular H.

Particularly preferred are compounds of general formula (I), wherein
R⁶ represents H.

Particularly preferred are compounds of general formula (I), wherein
R³ represents H;
R⁵ represents H; and
R⁶ represents H.

In accordance with a further aspect, the present invention covers compounds of general formula (I), wherein
R² represents
   —(CH₂)ₚ—(C₅-C₇-cycloalkyl),
   —(CH₂)ₚ-phenyl,
   5- or 6-membered heteroaryl wherein said 5-membered heteroaryl contains 1, 2 or 3 heteroatoms or heteroatom-containing groups independently selected from the group consisting of S, N, NH, and O, and wherein said 6-membered heteroaryl contains 1 or 2 N, or
   bicyclic 8- to 10-membered heteroaryl, containing 1, 2 or 3 heteroatoms or heteroatom-containing groups independently selected from NH, N, O, S, SO and SO₂,
   wherein said R² is optionally substituted at one or more carbon atoms with 1 to 3 substituents R^{2a} which are the same or different wherein R^{2a} represents C₁-C₅-alkyl, C₃-C₇-cycloalkyl, —(C₁-C₃-alkyl)-(C₃-C₇-cycloalkyl), —OC₁-C₅-alkyl, —OC₃-C₇-cycloalkyl, halogen, OH or CN, and
   wherein independently, if R² represents 5-membered heteroaryl or bicyclic 8- to 10-membered heteroaryl, each ring nitrogen atom, if present, of said R² is optionally substituted with a substituent R^{2b} wherein R^{2b} represents C₁-C₅-alkyl, C₃-C₇-cycloalkyl or —(C₁-C₃-alkyl)-(C₃-C₇-cycloalkyl), and
   if R^{2a} represents C₁-C₅-alkyl, C₃-C₇-cycloalkyl, —(C₁-C₃-alkyl)-(C₃-C₇-cycloalkyl), —OC₁-C₅-alkyl or —OC₃-C₇-cycloalkyl and/or if R^{2b} represents C₁-C₅-alkyl, C₃-C₇-cycloalkyl or —(C₁-C₃-alkyl)-(C₃-C₇-cycloalkyl),
   said C₁-C₅-alkyl, C₃-C₇-cycloalkyl, —(C₁-C₃-alkyl)-(C₃-C₇-cycloalkyl), —OC₁-C₅-alkyl and —OC₃-C₇-cycloalkyl independently are optionally substituted with one or more substituents independently selected from the group consisting of OH, OR⁴, and 1 to 5 fluorine atoms.

A preferred embodiment of the present invention covers compounds of general formula (I), wherein
R² represents —(CH₂)ₚ-phenyl, optionally substituted at one or more carbon atoms with 1 or 2 substituents R^{2a} which are the same or different wherein R^{2a} represents C₁-C₅-alkyl, C₃-C₇-cycloalkyl, OC₁-C₅-alkyl, halogen or CN,
   wherein said C₁-C₅-alkyl, —OC₁-C₅-alkyl and C₃-C₇-cycloalkyl independently are optionally substituted with OH, OR⁴ or 1 to 5 fluorine atoms; and
p and R⁴ have the same meaning as defined in general formula (I).

Also preferred are compounds of general formula (I), wherein
R² represents phenyl optionally substituted with 1 or 2 substituents R^{2a} which are the same or different wherein R^{2a} represents C₁-C₅-alkyl, C₃-C₇-cycloalkyl, OC₁-C₅-alkyl, halogen or CN,
   wherein said C₁-C₅-alkyl, —OC₁-C₅-alkyl and C₃-C₇-cycloalkyl independently are optionally substituted with OH, OR⁴ or 1 to 5 fluorine atoms, and
R⁴ has the same meaning as defined in general formula (I).

Also preferred are compounds of general formula (I), wherein
R² represents phenyl optionally substituted with 1 or 2 substituents R^{2a} which are the same or different wherein R^{2a} represents C₁-C₅-alkyl, OC₁-C₅-alkyl or Cl,
   wherein said C₁-C₅-alkyl and —OC₁-C₅-alkyl independently are optionally substituted with OH, OR⁴ or 1 to 5 fluorine atoms, and
R⁴ has the same meaning as defined in general formula (I).

Also preferred are compounds of general formula (I), wherein
R² represents phenyl substituted with 1 or 2 substituents R^{2a} which are the same or different wherein R^{2a} represents C₁-C₅-alkyl, OC₁-C₅-alkyl or Cl,
   wherein if the substituent or at least one of said substituents is C₁-C₅-alkyl, or Cl, it is preferably positioned para or meta to the carbon atom which links the phenyl to the rest of the molecule,
   wherein if the substituent or at least one of said substituents is OC₁-C₅-alkyl it is preferably positioned ortho to the carbon atom which links the phenyl to the rest of the molecule, and
   wherein said C₁-C₅-alkyl and —OC₁-C₅-alkyl independently are optionally substituted with 1 to 5 fluorine atoms.

Also preferred are compounds of general formula (I), wherein
R² represents phenyl substituted with 1 substituent R^{2a} selected from the group consisting of C₁-C₅-alkyl, OC₁-C₅-alkyl, F and Cl,
   wherein if the substituent is C₁-C₅-alkyl, or Cl, it is preferably positioned para or meta to the carbon atom which links the phenyl to the rest of the molecule,
   wherein if the substituent or at least one of said substituents is OC₁-C₅-alkyl it is preferably positioned ortho to the carbon atom which links the phenyl to the rest of the molecule, and
   wherein said C₁-C₅-alkyl and —OC₁-C₅-alkyl independently are optionally substituted with 1 to 5 fluorine atoms.

Also preferred are compounds of general formula (I), wherein
R² represents phenyl substituted with 1 substituent R^{2a} selected from the group consisting of methyl, trifluoromethyl, trifluoromethoxy, or Cl,
   wherein if the substituent is Cl, methyl, or trifluoromethyl it is preferably positioned para or meta to the carbon atom which links the phenyl to the rest of the molecule, and
   wherein if the substituent is trifluoromethoxy, it is preferably positioned ortho to the carbon atom which links the phenyl to the rest of the molecule.

Also preferred are compounds of general formula (I), wherein
R² represents phenyl substituted with trifluoromethyl and F,
   wherein the trifluoromethyl is preferably positioned para to the carbon atom which links the phenyl to the rest of the molecule, and
   wherein the F is preferably positioned ortho to the carbon atom which links the phenyl to the rest of the molecule.

In accordance with a further aspect, the present invention covers compounds of general formula (I), wherein
p represents 0.

In accordance with a further aspect, the present invention covers compounds of general formula (I), wherein
R⁴ represents C₁-C₅-alkyl, in particular methyl, ethyl, propyl, or butyl optionally substituted with 1-3 fluorine atoms.

Also preferred are compounds of general formula (I), wherein
R⁴ represents methyl, difluoromethyl or trifluoromethyl.

In accordance with a further aspect, the present invention covers compounds of general formula (I), wherein
R⁷ represents H or CH₃, in particular H; and
R⁸ represents H or CH₃, in particular H.

Also preferred are compounds of general formula (I), wherein
R⁷ represents H; and
R⁸ represents H.
R⁷ and R⁸ may be in cis or trans configuration, in particular in trans. Unless otherwise stated, it is referred to a mixture of enantiomers.

In accordance with a further aspect, the present invention covers compounds of general formula (I), wherein
R¹ represents 5- or 6-membered heteroaryl, wherein said 5-membered heteroaryl contains 1, 2 or 3 heteroatoms selected from the group consisting of S, N, and O, and wherein said 6-membered heteroaryl contains 1 or 2 N,
   wherein R¹ is optionally substituted as defined in formula (I);
R³ represents H or fluoro, in particular H;
R⁵ represents H, fluoro, chloro or methyl, in particular H;
R⁶ represents H, fluoro or OH, in particular H,
R⁷ represents H or CH₃, in particular H; and
R⁸ represents H or CH₃, in particular H.

In accordance with a further aspect, the present invention covers compounds of general formula (I), wherein
R¹ represents a 6-membered heteroaryl containing 1 or 2 N, in particular pyridinyl, pyrimidinyl or pyrazinyl,
   wherein said R¹ is optionally substituted at one or more carbon atoms with 1 to 3 substituents R^{1a} which are the same or different wherein R^{1a} represents C₁-C₅-alkyl, C₃-C₅-cycloalkyl, —(C₁-C₃-alkyl)-(C₃-C₅-cycloalkyl), —OC₁-C₅-alkyl, —OC₃-C₅-cycloalkyl, or halogen, and
   if R^{1a} represents C₁-C₅-alkyl, C₃-C₅-cycloalkyl, —(C₁-C₃-alkyl)-(C₃-C₅-cycloalkyl), —OC₁-C₅-alkyl or —OC₃-C₅-cycloalkyl, said C₁-C₅-alkyl, C₃-C₅-cycloalkyl, —(C₁-C₃-alkyl)-(C₃-C₅-cycloalkyl), —OC₁-C₅-alkyl and —OC₃-C₅-cycloalkyl independently are optionally substituted with one or more substituents independently selected from the group consisting of OH, OR⁴ and F; R⁴ has the same meaning as defined above in general formula (I);
R³ represents H or fluoro, in particular H;
R⁵ represents H, fluoro, chloro or methyl, in particular H;
R⁶ represents H, fluoro or OH, in particular H;
R⁷ represents H or CH₃, in particular H; and
R⁸ represents H or CH₃, in particular H.

In accordance with another aspect, the present invention covers compounds of general formula (I), wherein
R¹ represents 5-membered heteroaryl wherein said 5-membered heteroaryl contains 1, 2 or 3 heteroatoms or heteroatom-containing groups independently selected from the group consisting of S, N, and O, in particular pyrazolyl, thiazolyl, imidazolyl, or thiophenyl, wherein said R¹ is optionally substituted at one or more carbon atoms with 1 or 2 substituents R^{1a} which are the same or different, wherein R^{1a} represents C₁-C₅-alkyl, C₃-C₇-cycloalkyl, —(C₁-C₃-alkyl)-(C₃-C₇-cycloalkyl), —OC₁-C₅-alkyl, —OC₃-C₇-cycloalkyl, halogen, or CN, and
   wherein independently each ring nitrogen atom, if present, of said R¹ is optionally substituted with a substituent R^{1b}, wherein R^{1b} represents C₁-C₅-alkyl, —(C₁-C₃-alkyl)-(C₃-C₇-cycloalkyl), or C₃-C₇-cycloalkyl, and
      if R^{1a} represents C₁-C₅-alkyl, C₃-C₇-cycloalkyl, —(C₁-C₃-alkyl)-(C₃-C₇-cycloalkyl), —OC₁-C₅-alkyl, or —OC₃-C₇-cycloalkyl, and/or if R^{1b} represents C₁-C₅-alkyl, —(C₁-C₃-alkyl)-(C₃-C₇-cycloalkyl) or C₃-C₇-cycloalkyl, said C₁-C₅-alkyl, C₃-C₇-cycloalkyl, —(C₁-C₃-alkyl)-(C₃-C₇-cycloalkyl), —OC₁-C₅-alkyl, and —OC₃-C₇-cycloalkyl independently are optionally substituted with one or more substituents independently selected from the group consisting of methyl, ethyl, OH, OR⁴ and F; and wherein
R⁴ has the same meaning as defined above in general formula (I);
R³ represents H or fluoro, in particular H;
R⁵ represents H, fluoro, chloro or methyl, in particular H;
R⁶ represents H, fluoro or OH, in particular H;
R⁷ represents H or CH₃, in particular H; and
R⁸ represents H or CH₃, in particular H.

In accordance with another aspect, the present invention covers compounds of general formula (I), wherein
R¹ represents pyridinyl, in particular pyridin-3-yl,
   wherein said R¹ is optionally substituted at one or more carbon atoms with 1 to 3 substituents R^{1a} independently selected from the group consisting of C₁-C₅-alkyl, C₃-C₅-cycloalkyl, —(C₁-C₃-alkyl)-(C₃-C₅-cycloalkyl), —OC₁-C₅-alkyl, —OC₃-C₅-cycloalkyl, and halogen, and
   wherein one of said substituents is preferably positioned para to the atom to which the pyridinyl is attached to the rest of the molecule, more preferably attached to the carbon atom at the 6-position of pyridin-3-yl; and
   wherein said C₁-C₅-alkyl, C₃-C₇-cycloalkyl, —(C₁-C₃-alkyl)-(C₃-C₇-cycloalkyl), —OC₁-C₅-alkyl and —OC₃-C₇-cycloalkyl independently are optionally substituted with one or more substituents independently selected from the group consisting of OH, OR⁴ and F, in particular F;
R⁴ has the same meaning as defined above in general formula (I);
R³ represents H or fluoro, in particular H;
R⁵ represents H, fluoro, chloro or methyl, in particular H;
R⁶ represents H, fluoro or OH, in particular H;
R⁷ represents H or CH₃, in particular H; and
R⁸ represents H or CH₃, in particular H.

In accordance with another aspect, the present invention covers compounds of general formula (I), wherein
R¹ represents pyridinyl, in particular pyridin-3-yl,
   wherein said R¹ is substituted at the carbon atom positioned para to the atom to which the pyridinyl is attached to the rest of the molecule with a C₁-C₅-alkyl, more preferably attached to the carbon atom at the 6-position of pyridin-3-yl, and
   wherein said C₁-C₅-alkyl is optionally substituted with one or more substituents independently selected from the group consisting of OH, OR⁴ and F, in particular F;
R⁴ has the same meaning as defined above in general formula (I);
R³ represents H or fluoro, in particular H;
R⁵ represents H, fluoro, chloro or methyl, in particular H;
R⁶ represents H, fluoro or OH, in particular H;
R⁷ represents H or CH₃, in particular H; and
R⁸ represents H or CH₃, in particular H.

In accordance with another aspect, the present invention covers compounds of general formula (I), wherein
R¹ represents pyrazolyl, in particular pyrazol-4-yl, optionally substituted at one or more carbon atoms with 1 or 2 substituents R^{1a} which are the same or different, wherein R^{1a} represents C₁-C₅-alkyl, C₃-C₇-cycloalkyl, —(C₁-C₃-alkyl)-(C₃-C₇-cycloalkyl), —OC₁-C₅-alkyl, —OC₃-C₇-cycloalkyl, halogen, or CN, and
   wherein independently each ring nitrogen atom of said R¹ is optionally substituted with a substituent R^{1b}, wherein R^{1b} represents C₁-C₅-alkyl, —(C₁-C₃-alkyl)-(C₃-C₇-cycloalkyl), or C₃-C₇-cycloalkyl, and
   if R^{1a} represents C₁-C₅-alkyl, C₃-C₇-cycloalkyl, —(C₁-C₃-alkyl)-(C₃-C₇-cycloalkyl), —OC₁-C₅-alkyl, or —OC₃-C₇-cycloalkyl, and/or if R^{1b} represents C₁-C₅-alkyl, —(C₁-C₃-alkyl)-(C₃-C₇-cycloalkyl), or C₃-C₇-cycloalkyl, said C₁-C₅-alkyl, C₃-C₇-cycloalkyl, —(C₁-C₃-alkyl)-(C₃-C₇-cycloalkyl), —OC₁-C₅-alkyl and —OC₃-C₇-cycloalkyl independently are optionally substituted with one or more substituents independently selected from the group consisting of methyl, ethyl, OH, OR⁴ and F;
R⁴ has the same meaning as defined above in general formula (I);
R³ represents H or fluoro, in particular H;
R⁵ represents H, fluoro, chloro or methyl, in particular H;
R⁶ represents H, fluoro or OH, in particular H;
R⁷ represents H or CH₃, in particular H; and
R⁸ represents H or CH₃, in particular H.

A preferred embodiment of the present invention covers compounds of general formula (I), wherein
R¹ represents pyrazol-4-yl substituted at the nitrogen atom at position 1 with a substituent R^{1b}, wherein R^{1b} represents C₁-C₅-alkyl, —(C₁-C₃-alkyl)-(C₃-C₇-cycloalkyl) or C₃-C₇-cycloalkyl, wherein said C₁-C₅-alkyl, C₃-C₇-cycloalkyl and —(C₁-C₃-alkyl)-(C₃-C₇-cycloalkyl) are optionally substituted with one or more substituents independently selected from the group consisting of methyl, ethyl, OH, OR⁴ and F; and
R⁴ has the same meaning as defined above in general formula (I);
R³ represents H or fluoro, in particular H;
R⁵ represents H, fluoro, chloro or methyl, in particular H;
R⁶ represents H, fluoro or OH, in particular H;
R⁷ represents H or CH₃, in particular H; and
R⁸ represents H or CH₃, in particular H.

A preferred embodiment of the present invention covers compounds of general formula (I), wherein
R¹ represents pyrazol-4-yl substituted at the nitrogen atom at position 1 with C₃-C₇-cycloalkyl, wherein said C₃-C₇-cycloalkyl is optionally substituted with one or more substituents independently selected from the group consisting of methyl, ethyl, OH, OR⁴, and F;
R⁴ has the same meaning as defined above in general formula (I);
R³ represents H or fluoro, in particular H;
R⁵ represents H, fluoro, chloro or methyl, in particular H;
R⁶ represents H, fluoro or OH, in particular H;
R⁷ represents H or CH₃, in particular H; and
R⁸ represents H or CH₃, in particular H.

A preferred embodiment of the present invention covers compounds of general formula (I), wherein
R¹ represents pyrazol-4-yl, substituted at the nitrogen atom at position 1 with cyclobutyl;
R³ represents H or fluoro, in particular H;
R⁵ represents H, fluoro, chloro or methyl, in particular H;
R⁶ represents H, fluoro or OH, in particular H;
R⁷ represents H or CH₃, in particular H; and
R⁸ represents H or CH₃, in particular H.

In accordance with a further aspect, the present invention covers compounds of general formula (I), wherein
R² represents —(CH₂)ₚ-phenyl, optionally substituted at one or more carbon atoms with 1 or 2 substituents R^{2a} which are the same or different wherein R^{2a} represents C₁-C₅-alkyl, C₃-C₇-cycloalkyl, OC₁-C₅-alkyl, halogen or CN,
   wherein said C₁-C₅-alkyl, —OC₁-C₅-alkyl and C₃-C₇-cycloalkyl independently are optionally substituted with OH, OR⁴ or 1 to 5 fluorine atoms, and
p and R⁴ have the same meaning as defined in general formula (I);
R³ represents H or fluoro, in particular H;
R⁵ represents H, fluoro, chloro or methyl, in particular H;
R⁶ represents H, fluoro or OH, in particular H;
R⁷ represents H or CH₃, in particular H; and
R⁸ represents H or CH₃, in particular H.

A preferred embodiment of the invention relates to compounds of general formula (I), wherein
R² represents phenyl optionally substituted with 1 or 2 substituents R^{2a} which are the same or different wherein R^{2a} represents C₁-C₅-alkyl, C₃-C₇-cycloalkyl, OC₁-C₅-alkyl, halogen or CN,
   wherein said C₁-C₅-alkyl, —OC₁-C₅-alkyl and C₃-C₇-cycloalkyl independently are optionally substituted with OH, OR⁴ or 1 to 5 fluorine atoms;
R⁴ has the same meaning as defined in general formula (I);
R³ represents H or fluoro, in particular H;
R⁵ represents H, fluoro, chloro or methyl, in particular H;
R⁶ represents H, fluoro or OH, in particular H;
R⁷ represents H or CH₃, in particular H; and
R⁸ represents H or CH₃, in particular H.

A preferred embodiment of the invention relates to compounds of general formula (I), wherein
R² represents phenyl substituted with 1 or 2 substituents R^{2a} which are the same or different
   wherein R^{2a} represents C₁-C₅-alkyl, OC₁-C₅-alkyl or Cl,
   wherein if the substituent or at least one of said substituents is C₁-C₅-alkyl, or Cl, it is preferably positioned para or meta to the carbon atom which links the phenyl to the rest of the molecule,
   wherein if the substituent or at least one of said substituents is OC₁-C₅-alkyl it is preferably positioned ortho to the carbon atom which links the phenyl to the rest of the molecule, and
   wherein said C₁-C₅-alkyl and —OC₁-C₅-alkyl independently are optionally substituted with 1 to 5 fluorine atoms,
R³ represents H or fluoro, in particular H;
R⁵ represents H, fluoro, chloro or methyl, in particular H;
R⁶ represents H, fluoro or OH, in particular H;
R⁷ represents H or CH₃, in particular H; and
R⁸ represents H or CH₃, in particular H.

A preferred embodiment of the invention relates to compounds of general formula (I), wherein
R² represents phenyl substituted with 1 substituent R^{2a} selected from the group consisting of methyl, trifluoromethyl, trifluoromethoxy, or Cl,
   wherein if the substituent is Cl, methyl, or trifluoromethyl it is preferably positioned para or meta to the carbon atom which links the phenyl to the rest of the molecule, and
   wherein if the substituent is trifluoromethoxy, it is preferably positioned ortho to the carbon atom which links the phenyl to the rest of the molecule,
R³ represents H or fluoro, in particular H;
R⁵ represents H, fluoro, chloro or methyl, in particular H;
R⁶ represents H, fluoro or OH, in particular H;
R⁷ represents H or CH₃, in particular H; and
R⁸ represents H or CH₃, in particular H.

A preferred embodiment of the invention relates to compounds of general formula (I), wherein
R² represents phenyl substituted with trifluoromethyl and F,
   wherein the trifluoromethyl is positioned para to the carbon atom which links the phenyl to the rest of the molecule, and
   wherein the F is positioned ortho to the carbon atom which links the phenyl to the rest of the molecule;
R³ represents H or fluoro, in particular H;
R⁵ represents H, fluoro, chloro or methyl, in particular H;
R⁶ represents H, fluoro or OH, in particular H;
R⁷ represents H or CH₃, in particular H; and
R⁸ represents H or CH₃, in particular H.

In accordance with a further aspect, the present invention covers compounds of general formula (I), wherein
R¹ represents 6-membered heteroaryl containing 1 or 2 N, in particular pyridinyl, pyrimidinyl or pyrazinyl,
   wherein said R¹ is optionally substituted at one or more carbon atoms with 1 to 3 substituents R^{1a} which are the same or different wherein R^{1a} represents C₁-C₅-alkyl, C₃-C₅-cycloalkyl, —(C₁-C₃-alkyl)-(C₃-C₅-cycloalkyl), —OC₁-C₅-alkyl, —OC₃-C₅-cycloalkyl, or halogen, and
   if R^{1a} represents C₁-C₅-alkyl, C₃-C₅-cycloalkyl, —(C₁-C₃-alkyl)-(C₃-C₅-cycloalkyl), —OC₁-C₅-alkyl or —OC₃-C₅-cycloalkyl, said C₁-C₅-alkyl, C₃-C₅-cycloalkyl, —(C₁-C₃-alkyl)-(C₃-C₅-cycloalkyl), —OC₁-C₅-alkyl and —OC₃-C₅-cycloalkyl independently are optionally substituted with one or more substituents independently selected from the group consisting of OH, OR⁴ and F;
R² represents —(CH₂)ₚ-phenyl, optionally substituted at one or more carbon atoms with 1 or 2 substituents R^{2a} which are the same or different wherein R^{2a} represents C₁-C₅-alkyl, C₃-C₇-cycloalkyl, OC₁-C₅-alkyl, halogen or CN,
   wherein said C₁-C₅-alkyl, —OC₁-C₅-alkyl and C₃-C₇-cycloalkyl independently are optionally substituted with OH, OR⁴ or 1 to 5 fluorine atoms;
R³ represents H or fluoro, in particular H;
R⁵ represents H, fluoro, chloro or methyl, in particular H;
R⁶ represents H, fluoro or OH, in particular H;
R⁷ represents H or CH₃, in particular H;
R⁸ represents H or CH₃, in particular H; and
p and R⁴ have the same meaning as defined in general formula (I).

A preferred embodiment of the present invention covers compounds of general formula (I), wherein
R¹ represents pyridinyl, in particular pyridin-3-yl,
   wherein said R¹ is optionally substituted at one or more carbon atoms with 1 to 3 substituents R^{1a} independently selected from the group consisting of C₁-C₅-alkyl, C₃-C₅-cycloalkyl, —(C₁-C₃-alkyl)-(C₃-C₅-cycloalkyl), —OC₁-C₅-alkyl, —OC₃-C₅-cycloalkyl, or halogen, and
   wherein one of said substituents is preferably positioned para to the atom to which the pyridinyl is attached to the rest of the molecule, more preferably attached to the carbon atom at the 6-position of pyridin-3-yl; and
   wherein said C₁-C₅-alkyl, C₃-C₇-cycloalkyl, —(C₁-C₃-alkyl)-(C₃-C₇-cycloalkyl), —OC₁-C₅-alkyl and —OC₃-C₇-cycloalkyl independently are optionally substituted with one or more substituents independently selected from the group consisting of OH, OR⁴ and F, in particular F;
R² represents phenyl optionally substituted with 1 or 2 substituents R^{2a} which are the same or different wherein R^{2a} represents C₁-C₅-alkyl, C₃-C₇-cycloalkyl, OC₁-C₅-alkyl, halogen or CN,
   wherein said C₁-C₅-alkyl, —OC₁-C₅-alkyl and C₃-C₇-cycloalkyl independently are optionally substituted with OH, OR⁴ or 1 to 5 fluorine atoms;
R³ represents H or fluoro, in particular H;
R⁵ represents H, fluoro, chloro or methyl, in particular H;
R⁶ represents H, fluoro or OH, in particular H;
R⁷ represents H or CH₃, in particular H;
R⁸ represents H or CH₃, in particular H; and
R⁴ have the same meaning as defined in general formula (I).

A preferred embodiment of the present invention covers compounds of general formula (I), wherein
R¹ represents pyridinyl, in particular pyridin-3-yl,
   wherein said R¹ is substituted at the carbon atom positioned para to the atom to which the pyridinyl is attached to the rest of the molecule with a C₁-C₅-alkyl, more preferably attached to the carbon atom at the 6-position of pyridin-3-yl, and
   wherein said C₁-C₅-alkyl is optionally substituted with one or more substituents independently selected from the group consisting of OH, OR⁴ and F, in particular F,
R² represents phenyl optionally substituted with 1 or 2 substituents R^{2a} which are the same or different wherein R^{2a} represents C₁-C₅-alkyl, C₃-C₇-cycloalkyl, OC₁-C₅-alkyl, halogen or CN,
   wherein said C₁-C₅-alkyl, —OC₁-C₅-alkyl and C₃-C₇-cycloalkyl independently are optionally substituted with OH, OR⁴ or 1 to 5 fluorine atoms;
R³ represents H or fluoro, in particular H;
R⁵ represents H, fluoro, chloro or methyl, in particular H;
R⁶ represents H, fluoro or OH, in particular H;
R⁷ represents H or CH₃, in particular H;
R⁸ represents H or CH₃, in particular H; and
R⁴ has the same meaning as defined in general formula (I).

A preferred embodiment of the present invention covers compounds of general formula (I), wherein
R¹ represents pyridinyl, in particular pyridin-3-yl,
   wherein said R¹ is independently substituted at one or more carbon atoms with 1 or 2 C₁-C₅-alkyl substituents, which are the same or different, selected from the group consisting of methyl, ethyl, methoxy, ethoxy, trifluoromethyl, difluoromethyl, 1,1-difluoroethyl, 1,1-difluoropropyl and 2,2,2-trifluoroethyl, and
   wherein one of said substituents is preferably positioned para to the atom to which the pyridinyl is attached to the rest of the molecule, more preferably positioned at the carbon atom at the 6-position of pyridin-3-yl,
R² represents phenyl optionally substituted with 1 or 2 substituents R^{2a} which are the same or different wherein R^{2a} represents C₁-C₅-alkyl, C₃-C₇-cycloalkyl, OC₁-C₅-alkyl, halogen or CN,
   wherein said C₁-C₅-alkyl, —OC₁-C₅-alkyl and C₃-C₇-cycloalkyl independently are optionally substituted with OH, OR⁴ or 1 to 5 fluorine atoms;
R³ represents H or fluoro, in particular H;
R⁵ represents H, fluoro, chloro or methyl, in particular H;
R⁶ represents H, fluoro or OH, in particular H;
R⁷ represents H or CH₃, in particular H;
R⁸ represents H or CH₃, in particular H; and
R⁴ has the same meaning as defined in general formula (I).

A preferred embodiment of the present invention covers compounds of general formula (I), wherein

R¹ represents pyridinyl, in particular pyridin-3-yl,
wherein said R¹ is substituted with an optionally substituted C₁-C₅-alkyl substituent attached at the carbon atom positioned para to the atom to which the pyridinyl is attached to the rest of the molecule, in particular positioned at the carbon atom at the 6-position of pyridin-3-yl,
wherein said optionally substituted C₁-C₅-alkyl substituent is selected from the group consisting of methyl, ethyl, methoxy, ethoxy, trifluoromethyl, difluoromethyl, 1,1-difluoroethyl, 1,1-difluoropropyl and 2,2,2-trifluoroethyl;
R² represents phenyl optionally substituted with 1 or 2 substituents R^{2a} which are the same or different wherein R^{2a} represents C₁-C₅-alkyl, C₃-C₇-cycloalkyl, OC₁-C₅-alkyl, halogen or CN,
   wherein said C₁-C₅-alkyl, —OC₁-C₅-alkyl and C₃-C₇-cycloalkyl independently are optionally substituted with OH, OR⁴ or 1 to 5 fluorine atoms;
R³ represents H or fluoro, in particular H;
R⁵ represents H, fluoro, chloro or methyl, in particular H;
R⁶ represents H, fluoro or OH, in particular H;
R⁷ represents H or CH₃, in particular H;
R⁸ represents H or CH₃, in particular H; and
R⁴ has the same meaning as defined in general formula (I).

A preferred embodiment of the present invention covers compounds of general formula (I), wherein
R¹ represents pyridinyl, in particular pyridin-3-yl,
   wherein said R¹ is substituted with an optionally substituted C₁-C₅-alkyl substituent attached at the carbon atom positioned para to the atom to which the pyridinyl is attached to the rest of the molecule, in particular positioned at the carbon atom at the 6-position of pyridin-3-yl,
   wherein said optionally substituted C₁-C₅-alkyl substituent is selected from the group consisting of methyl, ethyl, methoxy, ethoxy, trifluoromethyl, difluoromethyl, 1,1-difluoroethyl, 1,1-difluoropropyl and 2,2,2-trifluoroethyl;
R² represents phenyl substituted with 1 substituent R^{2a} selected from the group consisting of methyl, trifluoromethyl, trifluoromethoxy, or Cl,
   wherein if the substituent is Cl, methyl, or trifluoromethyl it is preferably positioned para or meta to the carbon atom which links the phenyl to the rest of the molecule, and
   wherein if the substituent is trifluoromethoxy, it is preferably positioned ortho to the carbon atom which links the phenyl to the rest of the molecule,
R³ represents H or fluoro, in particular H;
R⁵ represents H, fluoro, chloro or methyl, in particular H;
R⁶ represents H, fluoro or OH, in particular H;
R⁷ represents H or CH₃, in particular H; and
R⁸ represents H or CH₃, in particular H.

A preferred embodiment of the present invention covers compounds of general formula (I), wherein
R¹ represents 5-membered heteroaryl wherein said 5-membered heteroaryl contains 1, 2 or 3 heteroatoms or heteroatom-containing groups independently selected from the group consisting of S, N, NH, and O, in particular pyrazolyl, thiazolyl, imidazolyl, or thiophenyl,
   wherein said R¹ is optionally substituted at one or more carbon atoms with 1 or 2 substituents R^{1a} which are the same or different, wherein R^{1a} represents C₁-C₅-alkyl, C₃-C₇-cycloalkyl, —(C₁-C₃-alkyl)-(C₃-C₇-cycloalkyl), —OC₁-C₅-alkyl, —OC₃-C₇-cycloalkyl, halogen, or CN, and
   wherein independently each ring nitrogen atom, if present, of said R¹ is optionally substituted with a substituent R^{1b}, wherein R^{1b} represents C₁-C₅-alkyl, —(C₁-C₃-alkyl)-(C₃-C₇-cycloalkyl), or C₃-C₇-cycloalkyl, and
   if R^{1a} represents C₁-C₅-alkyl, C₃-C₇-cycloalkyl, —(C₁-C₃-alkyl)-(C₃-C₇-cycloalkyl), —OC₁-C₅-alkyl, or —OC₃-C₇-cycloalkyl, and/or if R^{1b} represents C₁-C₅-alkyl, —(C₁-C₃-alkyl)-(C₃-C₇-cycloalkyl) or C₃-C₇-cycloalkyl, said C₁-C₅-alkyl, C₃-C₇-cycloalkyl, —(C₁-C₃-alkyl)-(C₃-C₇-cycloalkyl), —OC₁-C₅-alkyl, and —OC₃-C₇-cycloalkyl independently are optionally substituted with one or more substituents independently selected from the group consisting of methyl, ethyl, OH, OR⁴ and F,
R² represents —(CH₂)ₚ-phenyl, optionally substituted at one or more carbon atoms with 1 or 2 substituents R^{2a} which are the same or different wherein R^{2a} represents C₁-C₅-alkyl, C₃-C₇-cycloalkyl, OC₁-C₅-alkyl, halogen or CN,
   wherein said C₁-C₅-alkyl, —OC₁-C₅-alkyl and C₃-C₇-cycloalkyl independently are optionally substituted with OH, OR⁴ or 1 to 5 fluorine atoms;
R³ represents H or fluoro, in particular H;
R⁵ represents H, fluoro, chloro or methyl, in particular H;
R⁶ represents H, fluoro or OH, in particular H;
R⁷ represents H or CH₃, in particular H;
R⁸ represents H or CH₃, in particular H; and
p and R⁴ have the same meaning as defined in general formula (I).

A preferred embodiment of the present invention covers compounds of general formula (I), wherein
R¹ represents pyrazolyl, in particular pyrazol-4-yl, optionally substituted at one or more carbon atoms with 1 or 2 substituents R^{1a} which are the same or different, wherein R^{1a} represents C₁-C₅-alkyl, C₃-C₇-cycloalkyl, —(C₁-C₃-alkyl)-(C₃-C₇-cycloalkyl), —OC₁-C₅-alkyl, —OC₃-C₇-cycloalkyl, halogen, or CN, and
   wherein independently each ring nitrogen atom of said R¹ is optionally substituted with a substituent R^{1b}, wherein R^{1b} represents C₁-C₅-alkyl, —(C₁-C₃-alkyl)-(C₃-C₇-cycloalkyl), or C₃-C₇-cycloalkyl, and
   if R^{1a} represents C₁-C₅-alkyl, C₃-C₇-cycloalkyl, —(C₁-C₃-alkyl)-(C₃-C₇-cycloalkyl), —OC₁-C₅-alkyl, or —OC₃-C₇-cycloalkyl, and/or if R^{1b} represents C₁-C₅-alkyl, —(C₁-C₃-alkyl)-(C₃-C₇-cycloalkyl), or C₃-C₇-cycloalkyl, said C₁-C₅-alkyl, C₃-C₇-cycloalkyl, —(C₁-C₃-alkyl)-(C₃-C₇-cycloalkyl), —OC₁-C₅-alkyl and —OC₃-C₇-cycloalkyl independently are optionally substituted with one or more substituents independently selected from the group consisting of methyl, ethyl, OH, OR⁴ and F,
R² represents phenyl substituted with 1 or 2 substituents which are the same or different selected from the group consisting of C₁-C₅-alkyl, OC₁-C₅-alkyl, fluoro and chloro,
   wherein said C₁-C₅-alkyl and OC₁-C₅-alkyl are optionally substituted with 1 to 5 fluorine atoms;
R³ represents H or fluoro, in particular H;
R⁵ represents H, fluoro, chloro or methyl, in particular H;
R⁶ represents H, fluoro or OH, in particular H;
R⁷ represents H or CH₃, in particular H;
R⁸ represents H or CH₃, in particular H; and
R⁴ has the same meaning as defined in general formula (I).

A preferred embodiment of the present invention covers compounds of general formula (I), wherein
R¹ represents pyrazol-4-yl substituted at the nitrogen atom at position 1 with a substituent R^{1b}, wherein R^{1b} represents C₁-C₅-alkyl, —(C₁-C₃-alkyl)-(C₃-C₇-cycloalkyl) or C₃-C₇-cycloalkyl, wherein said C₁-C₅-alkyl, C₃-C₇-cycloalkyl and —(C₁-C₃-alkyl)-(C₃-C₇-cycloalkyl) is optionally substituted with one or more substituents independently selected from the group consisting of methyl, ethyl, OH, OR⁴ and F;
R² represents phenyl substituted with 1 or 2 substituents which are the same or different selected from the group consisting of C₁-C₅-alkyl, OC₁-C₅-alkyl, fluoro and chloro,
   wherein said C₁-C₅-alkyl and OC₁-C₅-alkyl are optionally substituted with 1 to 5 fluorine atoms;
R³ represents H or fluoro, in particular H;
R⁵ represents H, fluoro, chloro or methyl, in particular H;
R⁶ represents H, fluoro or OH, in particular H;
R⁷ represents H or CH₃, in particular H;
R⁸ represents H or CH₃, in particular H; and
R⁴ has the same meaning as defined in general formula (I).

A preferred embodiment of the present invention covers compounds of general formula (I), wherein
R¹ represents pyrazolyl, in particular pyrazol-4-yl, wherein said R¹ is optionally substituted with 1 or 2 R^{1b} which are the same or different, wherein R^{1b} represents C₁-C₅-alkyl, C₃-C₇-cycloalkyl or —(C₁-C₃-alkyl)-(C₃-C₇-cycloalkyl), and
   wherein one of said substituents R^{1b} is attached to the pyrazolyl nitrogen atom at position 1, preferably attached to the pyrazol-4-yl nitrogen atom at position 1, and wherein said C₁-C₅-alkyl, C₃-C₇-cycloalkyl and —(C₁-C₃-alkyl)-(C₃-C₇-cycloalkyl) independently are optionally substituted with one or more substituents independently selected from the group consisting of methyl, ethyl, OH, OR⁴, and F,
R² represents phenyl substituted with 1 or 2 substituents which are the same or different selected from the group consisting of C₁-C₅-alkyl, OC₁-C₅-alkyl, fluoro and chloro,
   wherein said C₁-C₅-alkyl and OC₁-C₅-alkyl are optionally substituted with 1 to 5 fluorine atoms;
R³ represents H or fluoro, in particular H;
R⁵ represents H, fluoro, chloro or methyl, in particular H;
R⁶ represents H, fluoro or OH, in particular H;
R⁷ represents H or CH₃, in particular H;
R⁸ represents H or CH₃, in particular H; and
R⁴ has the same meaning as defined in general formula (I).

A preferred embodiment of the present invention covers compounds of general formula (I), wherein
R¹ represents pyrazol-4-yl substituted at the nitrogen atom at position 1 with C₃-C₇-cycloalkyl, wherein said C₃-C₇-cycloalkyl is optionally substituted with one or more substituents independently selected from the group consisting of methyl, ethyl, OH, OR⁴, and F, in particular R¹ represents pyrazol-4-yl substituted with cyclobutyl at position 1;
R² represents phenyl substituted with 1 substituent R^{2a} selected from the group consisting of methyl, trifluoromethyl, trifluoromethoxy, or Cl,
   wherein if the substituent is Cl, methyl, or trifluoromethyl it is preferably positioned para or meta to the carbon atom which links the phenyl to the rest of the molecule, and
   wherein if the substituent is trifluoromethoxy, it is preferably positioned ortho to the carbon atom which links the phenyl to the rest of the molecule,
R³ represents H or fluoro, in particular H;
R⁵ represents H, fluoro, chloro or methyl, in particular H;
R⁶ represents H, fluoro or OH, in particular H;
R⁷ represents H or CH₃, in particular H; and
R⁸ represents H or CH₃, in particular H.

A preferred embodiment of the present invention covers compounds of general formula (I), wherein
R¹ represents pyrazol-4-yl substituted at the nitrogen atom at position 1 with C₃-C₇-cycloalkyl, wherein said C₃-C₇-cycloalkyl is optionally substituted with one or more substituents independently selected from the group consisting of methyl, ethyl, OH, OR⁴, and F;
R² represents phenyl substituted with trifluoromethyl and F,
   wherein the trifluoromethyl is preferably positioned para to the carbon atom which links the phenyl to the rest of the molecule, and
   wherein the F is preferably positioned ortho to the carbon atom which links the phenyl to the rest of the molecule;
R³ represents H or fluoro, in particular H;
R⁵ represents H, fluoro, chloro or methyl, in particular H;
R⁶ represents H, fluoro or OH, in particular H;
R⁷ represents H or CH₃, in particular H;
R⁸ represents H or CH₃, in particular H; and
R⁴ has the same meaning as defined in general formula (I).

A preferred embodiment of the present invention covers compounds of general formula (I), wherein
R¹ represents pyrazol-4-yl substituted at the nitrogen atom at position 1 with cyclobutyl;
R² represents phenyl substituted with trifluoromethyl and F,
   wherein the trifluoromethyl is positioned para to the carbon atom which links the phenyl to the rest of the molecule, and
   wherein the F is positioned ortho to the carbon atom which links the phenyl to the rest of the molecule;
R³ represents H;
R⁵ represents H;
R⁶ represents H;
R⁷ represents H;
R⁸ represents H; and

wherein R⁷ and R⁸ are in trans or cis configuration, in particular in trans configuration.

Most preferred compounds are, namely selected from the group consisting of:
5-({[trans-2-(3-chlorophenyl)cyclopropyl]carbonyl}amino)-2-(1-cyclobutyl-1H-pyrazol-4-yl)benzoic acid;
5-({[trans-2-(4-chlorophenyl)cyclopropyl]carbonyl}amino)-2-(1-cyclobutyl-1H-pyrazol-4-yl)-3-fluorobenzoic acid;
(+)-5-({[trans-2-(4-chlorophenyl)cyclopropyl]carbonyl}amino)-2-(1-cyclobutyl-1H-pyrazol-4-yl)-3-fluorobenzoic acid;
(−)-5-({[trans-2-(4-chlorophenyl)cyclopropyl]carbonyl}amino)-2-(1-cyclobutyl-1H-pyrazol-4-yl)-3-fluorobenzoic acid;
2-(1-cyclobutyl-1H-pyrazol-4-yl)-5-({[trans-2-(3-methylphenyl)cyclopropyl]carbonyl}amino) benzoic acid;
5-({[trans-2-(4-chlorophenyl)cyclopropyl]carbonyl}amino)-2-(1-cyclobutyl-1H-pyrazol-4-yl)benzoic acid;
(+)-5-({[trans-2-(3-chlorophenyl)cyclopropyl]carbonyl}amino)-2-(1-cyclobutyl-1H-pyrazol-4-yl)-3-fluorobenzoic acid;
(−)-5-({[trans-2-(3-chlorophenyl)cyclopropyl]carbonyl}amino)-2-(1-cyclobutyl-1H-pyrazol-4-yl)-3-fluorobenzoic acid;
2-(1-cyclobutyl-1H-pyrazol-4-yl)-5-[({trans-2-[2-(trifluoromethoxy)phenyl]cyclopropyl} carbonyl)amino]benzoic acid;
2-(1-cyclobutyl-1H-pyrazol-4-yl)-5-[({trans-2-[3-(trifluoromethyl)phenyl]cyclopropyl} carbonyl)amino]benzoic acid;
2-(4-tert-butyl-1H-pyrazol-1-yl)-5-({[trans-2-(4-chlorophenyl)cyclopropyl]carbonyl} amino)benzoic acid;
2-(3-tert-butyl-1H-pyrazol-1-yl)-5-({[trans-2-(4-chlorophenyl)cyclopropyl]carbonyl} amino)benzoic acid;
5-({[trans-2-(4-chlorophenyl)cyclopropyl]carbonyl}amino)-2-(6-methylpyridin-3-yl)benzoic acid;
2-[6-(1,1-difluoroethyl)pyridin-3-yl]-5-[({trans-2-[2-(trifluoromethoxy)phenyl]cyclopropyl} carbonyl)amino]benzoic acid;
5-({[trans-2-(3-chlorophenyl)cyclopropyl]carbonyl}amino)-2-[6-(1,1-difluoroethyl)pyridin-3-yl]benzoic acid;
2-[6-(1,1-difluoroethyl)pyridin-3-yl]-5-[({trans-2-[3-(trifluoromethyl)phenyl]cyclopropyl} carbonyl)amino]benzoic acid;
5-({[trans-2-(4-chlorophenyl)cyclopropyl]carbonyl}amino)-2-[6-(1,1-difluoroethyl)pyridin-3-yl]benzoic acid;
2-[6-(1,1-difluoropropyl)pyridin-3-yl]-5-[({trans-2-[2-(trifluoromethoxy)phenyl]cyclopropyl} carbonyl)amino]benzoic acid;
2-[6-(1,1-difluoropropyl)pyridin-3-yl]-5-[({trans-2-[3-(trifluoromethyl)phenyl]cyclopropyl} carbonyl)amino]benzoic acid;
5-({[trans-2-(3-chlorophenyl)cyclopropyl]carbonyl}amino)-2-[6-(1,1-difluoropropyl)pyridin-3-yl]benzoic acid;
5-({[trans-2-(4-chlorophenyl)cyclopropyl]carbonyl}amino)-2-[6-(1,1-difluoropropyl)pyridin-3-yl]benzoic acid;
5-({[trans-2-(4-chlorophenyl)cyclopropyl]carbonyl}amino)-3-fluoro-2-[6-(trifluoromethyl) pyridin-3-yl]benzoic acid; and
5-({[trans-2-(4-chlorophenyl)cyclopropyl]carbonyl}amino)-4-fluoro-2-[6-(trifluoromethyl) pyridin-3-yl]benzoic acid;

or an isomer, enantiomer, diastereomer, racemate, hydrate, solvate, or a salt thereof, or a mixture of same.

Pharmaceutical Compositions of the Compounds of the Invention

It is possible for the compounds according to the invention to have systemic and/or local activity. For this purpose, they can be administered in a suitable manner, such as, for example, via the oral, parenteral, pulmonary, nasal, sublingual, lingual, buccal, rectal, vaginal, dermal, transdermal, conjunctival, otic route or as an implant or stent.

For these administration routes, it is possible for the compounds according to the invention to be administered in suitable administration forms.

For oral administration, it is possible to formulate the compounds according to the invention to dosage forms known in the art that deliver the compounds of the invention rapidly and/or in a modified manner, such as, for example, tablets (uncoated or coated tablets, for example with enteric or controlled release coatings that dissolve with a delay or are insoluble), orally-disintegrating tablets, films/wafers, films/lyophylisates, capsules (for example hard or soft gelatine capsules), sugar-coated tablets, granules, pellets, powders, emulsions, suspensions, aerosols or solutions. It is possible to incorporate the compounds according to the invention in crystalline and/or amorphised and/or dissolved form into said dosage forms.

Parenteral administration can be effected with avoidance of an absorption step (for example intravenous, intraarterial, intracardial, intraspinal or intralumbal) or with inclusion of absorption (for example intramuscular, subcutaneous, intracutaneous, percutaneous or intraperitoneal). Administration forms which are suitable for parenteral administration are, inter alia, preparations for injection and infusion in the form of solutions, suspensions, emulsions, lyophylisates or sterile powders.

Examples which are suitable for other administration routes are pharmaceutical forms for inhalation [inter alia powder inhalers, nebulizers], nasal drops, nasal solutions, nasal sprays; tablets/films/wafers/capsules for lingual, sublingual or buccal administration; suppositories; eye drops, eye ointments, eye baths, ocular inserts, ear drops, ear sprays, ear powders, ear-rinses, ear tampons; vaginal capsules, aqueous suspensions (lotions, mixture agitandae), lipophilic suspensions, emulsions, ointments, creams, transdermal therapeutic systems (such as, for example, patches), milk, pastes, foams, dusting powders, implants or stents.

The compounds according to the invention can be incorporated into the stated administration forms. This can be effected in a manner known per se by mixing with pharmaceutically suitable excipients. Pharmaceutically suitable excipients include, inter alia,
fillers and carriers (for example cellulose, microcrystalline cellulose (such as, for example, Avicel®), lactose, mannitol, starch, calcium phosphate (such as, for example, Di-Cafos®)),
ointment bases (for example petroleum jelly, paraffins, triglycerides, waxes, wool wax, wool wax alcohols, lanolin, hydrophilic ointment, polyethylene glycols),
bases for suppositories (for example polyethylene glycols, cacao butter, hard fat),
solvents (for example water, ethanol, isopropanol, glycerol, propylene glycol, medium chain-length triglycerides fatty oils, liquid polyethylene glycols, paraffins),
surfactants, emulsifiers, dispersants or wetters (for example sodium dodecyl sulfate), lecithin, phospholipids, fatty alcohols (such as, for example, Lanette®), sorbitan fatty acid esters (such as, for example, Span®), polyoxyethylene sorbitan fatty acid esters (such as, for example, Tween®), polyoxyethylene fatty acid glycerides (such as, for example, Cremophor®), polyoxethylene fatty acid esters, polyoxyethylene fatty alcohol ethers, glycerol fatty acid esters, poloxamers (such as, for example, Pluronic®),
buffers, acids and bases (for example phosphates, carbonates, citric acid, acetic acid, hydrochloric acid, sodium hydroxide solution, ammonium carbonate, trometamol, triethanolamine),
isotonicity agents (for example glucose, sodium chloride),
adsorbents (for example highly-disperse silicas),
viscosity-increasing agents, gel formers, thickeners and/or binders (for example polyvinylpyrrolidone, methylcellulose, hydroxypropylmethylcellulose, hydroxypropylcellulose, carboxymethylcellulose-sodium, starch, carbomers, polyacrylic acids (such as, for example, Carbopol®); alginates, gelatine),
disintegrants (for example modified starch, carboxymethylcellulose-sodium, sodium starch glycolate (such as, for example, Explotab®), cross-linked polyvinylpyrrolidone, croscarmellose-sodium (such as, for example, AcDiSol®)),
flow regulators, lubricants, glidants and mould release agents (for example magnesium stearate, stearic acid, talc, highly-disperse silicas (such as, for example, Aerosil®)),
coating materials (for example sugar, shellac) and film formers for films or diffusion membranes which dissolve rapidly or in a modified manner (for example polyvinylpyrrolidones (such as, for example, Kollidon®), polyvinyl alcohol, hydroxypropylmethylcellulose, hydroxypropylcellulose, ethylcellulose, hydroxypropylmethylcellulose phthalate, cellulose acetate, cellulose acetate phthalate, polyacrylates, polymethacrylates such as, for example, Eudragit®)),
capsule materials (for example gelatine, hydroxypropylmethylcellulose),
synthetic polymers (for example polylactides, polyglycolides, poly acrylates, polymethacrylates (such as, for example, Eudragit®), polyvinylpyrrolidones (such as, for example, Kollidon®), polyvinyl alcohols, polyvinyl acetates, polyethylene oxides, polyethylene glycols and their copolymers and blockcopolymers),
plasticizers (for example polyethylene glycols, propylene glycol, glycerol, triacetine, triacetyl citrate, dibutyl phthalate),
penetration enhancers,
stabilisers (for example antioxidants such as, for example, ascorbic acid, ascorbyl palmitate, sodium ascorbate, butylhydroxyanisole, butylhydroxytoluene, propyl gallate),
preservatives (for example parabens, sorbic acid, thiomersal, benzalkonium chloride, chlorhexidine acetate, sodium benzoate),
colourants (for example inorganic pigments such as, for example, iron oxides, titanium dioxide),
flavourings, sweeteners, flavour- and/or odour-masking agents.

The present invention furthermore relates to a pharmaceutical composition which comprises at least one compound according to the invention, conventionally together with one or more pharmaceutically suitable excipient(s), and to their use according to the present invention.

Combination Therapies

The term “combination” in the present invention is used as known to persons skilled in the art and may be present as a fixed combination, a non-fixed combination or kit-of-parts.

A “fixed combination” in the present invention is used as known to persons skilled in the art and is defined as a combination wherein the said first active ingredient and the said second active ingredient are present together in one unit dosage or in a single entity. One example of a “fixed combination” is a pharmaceutical composition wherein the said first active ingredient and the said second active ingredient are present in admixture for simultaneous administration, such as in a formulation. Another example of a “fixed combination” is a pharmaceutical combination wherein the said first active ingredient and the said second active ingredient are present in one unit without being in admixture.

A non-fixed combination or “kit-of-parts” in the present invention is used as known to persons skilled in the art and is defined as a combination wherein the said first active ingredient and the said second active ingredient are present in more than one unit. One example of a non-fixed combination or kit-of-parts is a combination wherein the said first active ingredient and the said second active ingredient are present separately. The components of the non-fixed combination or kit-of-parts may be administered separately, sequentially, simultaneously, concurrently or chronologically staggered.

The compounds of this invention can be administered as the sole pharmaceutical agent or in combination with one or more other pharmaceutical agents where the combination causes no unacceptable adverse effects. The present invention relates also to such combinations.

For example, the compounds of this invention can be combined with known hormonal therapeutical agents.

In particular, the compounds of the present invention can be administered in combination or as comedication with hormonal contraceptives. Hormonal contraceptives are for example Combined Oral Contraceptives (COCs) or Progestin-Only-Pills (POPs) or hormone-containing devices.

COCs include but are not limited to birth control pills or a birth control method that includes a combination of an estrogen (estradiol) and a progestogen (progestin). The estrogenic part is in most of the COCs ethinyl estradiol. Some COCs contain estradiol or estradiol valerate.

Said COCs contain the progestins norethynodrel, norethindrone, norethindrone acetate, ethynodiol acetate, norgestrel, levonorgestrel, norgestimate, desogestrel, gestodene, drospirenone, dienogest, or nomegestrol acetate.

Birth control pills include for example but are not limited to Yasmin, Yaz, both containing ethinyl estradiol and drospirenone; Microgynon or Miranova containing levonorgestrel and ethinyl estradiol; Marvelon containing ethinyl estradiol and desogestrel; Valette containing ethinyl estradiol and dienogest; Belara and Enriqa containing ethinyl estradiol and chlormadinonacetate; Qlaira containing estradiol valerate and dienogest as active ingredients; and Zoely containing estradiol and normegestrol.

POPs are contraceptive pills that contain only synthetic progestogens (progestins) and do not contain estrogen. They are colloquially known as mini pills.

POPs include but are not limited to Cerazette containing desogestrel; and Micronor containing norethindrone.

Other Progeston-Only forms are intrauterine devices (IUDs), for example Mirena containing levonorgestrel or injectables, for example Depo-Provera containing medroxyprogesterone acetate, or implants, for example Implanon containing etonogestrel.

Other hormone-containing devices with contraceptive effect which are suitable for a combination with the compounds of the present invention are vaginal rings like Nuvaring containing ethinyl estradiol and etonogestrel, or transdermal systems like contraceptive patches, for example Ortho-Evra containing ethinyl estradiol and norelgestromin or Apleek (Lisvy) containing ethinyl estradiol and gestodene.

A preferred embodiment of the present invention is the administration of a compound of general formula (I) in combination with a COC or a POP or other Progestin-Only forms, as well as in combination with vaginal rings or contraceptive patches as mentioned above.

Furthermore, the compounds of the present invention can be combined with therapeutic agents or active ingredients, that are already approved or that are still under development for the treatment and/or prophylaxis of diseases which are related to or mediated by the Bradykinin B1 receptor.

For the treatment and/or prophylaxis of urinary tract diseases, the compounds of the present invention can be administered in combination or as co-medication with any substance that can be applied as therapeutic agent in the following indications:

Urinary tract disease states associated with the bladder outlet obstruction; urinary incontinence conditions such as reduced bladder capacity, increased frequency of micturition, urge incontinence, stress incontinence, or bladder hyperreactivity; benign prostatic hypertrophy; prostatic hyperplasia; prostatitis; detrusor hyperreflexia; overactive bladder and symptoms related to overactive bladder wherein said symptoms are in particular increased urinary frequency, nocturia, urinary urgency or urge incontinence; pelvic hypersensitivity; urethritis; prostatitis; prostatodynia; cystitis, in particular interstitial cystitis; idiopathic bladder hypersensitivity.

For the treatment and/or prophylaxis of overactive bladder and symptoms related to overactive bladder, the compounds of the present invention can be administered in combination or as co-medication in addition to behavioural therapy like diet, lifestyle or bladder training with anticholinergics like oxybutynin, tolterodine, propiverine, solifenacin, darifenacin, trospium, fesoterdine; ß-3 agonists like mirabegron; neurotoxins like onabutolinumtoxin A; or antidepressants like imipramine, duloxetine.

For the treatment and/or prophylaxis of interstitial cystitis, the compounds of the present invention can be administered in combination or as co-medication in addition to behavioural therapy like diet, lifestyle or bladder training with pentosans like elmiron; antidepressants like amitriptyline, imipramine; or antihistamines like loratadine.

For the treatment and/or prophylaxis of gynaecological diseases, the compounds of the present invention can be administered in combination or as co-medication with any substance that can be applied as therapeutic agent in the following indications:

dysmenorrhea, including primary and secondary; dyspareunia; endometriosis; endometriosis-associated pain; endometriosis-associated symptoms, such as and in particular dysmenorrhea, dyspareunia, dysuria, or dyschezia.

For the treatment and/or prophylaxis of dysmenorrhea, including primary and secondary; dyspareunia; endometriosis and endometriosis-associated pain, the compounds of the present invention can be administered in combination with ovulation inhibiting treatment, in particular COCs as mentioned above or contraceptive patches like Ortho-Evra or Apleek (Lisvy); or with progestogenes like dienogest (Visanne); or with GnRH analogous, in particular GnRH agonists and antagonists, for example leuprorelin, nafarelin, goserelin, cetrorelix, abarelix, ganirelix, degarelix; or with androgens: danazol.

For the treatment and/or prophylaxis of diseases, which are associated with pain, or pain syndromes, the compounds of the present invention can be administered in combination or as co-medication with any substance that can be applied as therapeutic agent in the following indications: pain-associated diseases or disorders like hyperalgesia, allodynia, functional bowel disorders (such as irritable bowel syndrome) and arthritis (such as osteoarthritis, rheumatoid arthritis and ankylosing spondylitis), burning mouth syndrome, burns, migraine or cluster headache, nerve injury, traumatic nerve injury, post-traumatic injuries (including fractures and sport injuries), neuritis, neuralgia, poisoning, ischemic injury, interstitial cystitis, viral, trigeminal neuralgia, small fiber neuropathy, diabetic neuropathy, chronic arthritis and related neuralgias, HIV and HIV treatment-induced neuropathy.

The compounds of the present invention can be combined with other pharmacological agents and compounds that are intended to treat inflammatory diseases, inflammatory pain or general pain conditions.

In addition to well-known medicaments which are already approved and on the market, the compounds of the present invention can be administered in combination with inhibitors of the P2X purinoceptor family (P2X3, P2X4), with inhibitors of IRAK4 and with antagonists of the prostanoid EP4 receptor.

In particular, the compounds of the present invention can be administered in combination with pharmacological endometriosis agents, intended to treat inflammatory diseases, inflammatory pain or general pain conditions and/or interfering with endometriotic proliferation and endometriosis associated symptoms, namely with inhibitors of Aldo-keto-reductase1C3 (AKR1C3) and with functional blocking antibodies of the prolactin receptor.

The compounds of the present invention can be combined with other pharmacological agents and compounds that are intended for the treatment, prevention or management of cancer.

In particular, the compounds of the present invention can be administered in combination with 131I-chTNT, abarelix, abiraterone, aclarubicin, ado-trastuzumab emtansine, afatinib, aflibercept, aldesleukin, alemtuzumab, Alendronic acid, alitretinoin, altretamine, amifostine, aminoglutethimide, Hexyl aminolevulinate, amrubicin, amsacrine, anastrozole, ancestim, anethole dithiolethione, angiotensin II, antithrombin III, aprepitant, arcitumomab, arglabin, arsenic trioxide, asparaginase, axitinib, azacitidine, basiliximab, belotecan, bendamustine, belinostat, bevacizumab, bexarotene, bicalutamide, bisantrene, bleomycin, bortezomib, buserelin, bosutinib, brentuximab vedotin, busulfan, cabazitaxel, cabozantinib, calcium folinate, calcium levofolinate, capecitabine, capromab, carboplatin, carfilzomib, carmofur, carmustine, catumaxomab, celecoxib, celmoleukin, ceritinib, cetuximab, chlorambucil, chlormadinone, chlormethine, cidofovir, cinacalcet, cisplatin, cladribine, clodronic acid, clofarabine, copanlisib, crisantaspase, cyclophosphamide, cyproterone, cytarabine, dacarbazine, dactinomycin, darbepoetin alfa, dabrafenib, dasatinib, daunorubicin, decitabine, degarelix, denileukin diftitox, denosumab, depreotide, deslorelin, dexrazoxane, dibrospidium chloride, dianhydrogalactitol, diclofenac, docetaxel, dolasetron, doxifluridine, doxorubicin, doxorubicin+estrone, dronabinol, eculizumab, edrecolomab, elliptinium acetate, eltrombopag, endostatin, enocitabine, enzalutamide, epirubicin, epitiostanol, epoetin alfa, epoetin beta, epoetin zeta, eptaplatin, eribulin, erlotinib, esomeprazole, estradiol, estramustine, etoposide, everolimus, exemestane, fadrozole, fentanyl, filgrastim, fluoxymesterone, floxuridine, fludarabine, fluorouracil, flutamide, folinic acid, formestane, fosaprepitant, fotemustine, fulvestrant, gadobutrol, gadoteridol, gadoteric acid meglumine, gadoversetamide, gadoxetic acid, gallium nitrate, ganirelix, gefitinib, gemcitabine, gemtuzumab, Glucarpidase, glutoxim, GM-CSF, goserelin, granisetron, granulocyte colony stimulating factor, histamine dihydrochloride, histrelin, hydroxycarbamide, I-125 seeds, lansoprazole, ibandronic acid, ibritumomab tiuxetan, ibrutinib, idarubicin, ifosfamide, imatinib, imiquimod, improsulfan, indisetron, incadronic acid, ingenol mebutate, interferon alfa, interferon beta, interferon gamma, iobitridol, iobenguane (123I), iomeprol, ipilimumab, irinotecan, Itraconazole, ixabepilone, lanreotide, lapatinib, lasocholine, lenalidomide, lenograstim, lentinan, letrozole, leuprorelin, levamisole, levonorgestrel, levothyroxine sodium, lisuride, lobaplatin, lomustine, lonidamine, masoprocol, medroxyprogesterone, megestrol, melarsoprol, melphalan, mepitiostane, mercaptopurine, mesna, methadone, methotrexate, methoxsalen, methylaminolevulinate, methylprednisolone, methyltestosterone, metirosine, mifamurtide, miltefosine, miriplatin, mitobronitol, mitoguazone, mitolactol, mitomycin, mitotane, mitoxantrone, mogamulizumab, molgramostim, mopidamol, morphine hydrochloride, morphine sulfate, nabilone, nabiximols, nafarelin, naloxone+pentazocine, naltrexone, nartograstim, nedaplatin, nelarabine, neridronic acid, nivolumabpentetreotide, nilotinib, nilutamide, nimorazole, nimotuzumab, nimustine, nitracrine, nivolumab, obinutuzumab, octreotide, ofatumumab, omacetaxine mepesuccinate, omeprazole, ondansetron, oprelvekin, orgotein, orilotimod, oxaliplatin, oxycodone, oxymetholone, ozogamicine, p53 gene therapy, paclitaxel, palifermin, palladium-103 seed, palonosetron, pamidronic acid, panitumumab, pantoprazole, pazopanib, pegaspargase, PEG-epoetin beta (methoxy PEG-epoetin beta), pembrolizumab, pegfilgrastim, peginterferon alfa-2b, pemetrexed, pentazocine, pentostatin, peplomycin, Perflubutane, perfosfamide, Pertuzumab, picibanil, pilocarpine, pirarubicin, pixantrone, plerixafor, plicamycin, poliglusam, polyestradiol phosphate, polyvinylpyrrolidone+sodium hyaluronate, polysaccharide-K, pomalidomide, ponatinib, porfimer sodium, pralatrexate, prednimustine, prednisone, procarbazine, procodazole, propranolol, quinagolide, rabeprazole, racotumomab, radium-223 chloride, radotinib, raloxifene, raltitrexed, ramosetron, ramucirumab, ranimustine, rasburicase, razoxane, refametinib, regorafenib, risedronic acid, rhenium-186 etidronate, rituximab, romidepsin, romiplostim, romurtide, roniciclib, samarium (153Sm) lexidronam, sargramostim, satumomab, secretin, sipuleucel-T, sizofiran, sobuzoxane, sodium glycididazole, sorafenib, stanozolol, streptozocin, sunitinib, talaporfin, tamibarotene, tamoxifen, tapentadol, tasonermin, teceleukin, technetium (99mTc) nofetumomab merpentan, 99mTc-HYNIC-[Tyr3]-octreotide, tegafur, tegafur+gimeracil+oteracil, temoporfin, temozolomide, temsirolimus, teniposide, testosterone, tetrofosmin, thalidomide, thiotepa, thymalfasin, thyrotropin alfa, tioguanine, tocilizumab, topotecan, toremifene, tositumomab, trabectedin, tramadol, trastuzumab, trastuzumab emtansine, treosulfan, tretinoin, trifluridine+tipiracil, trilostane, triptorelin, trametinib, trofosfamide, thrombopoietin, tryptophan, ubenimex, valatinib, valrubicin, vandetanib, vapreotide, vemurafenib, vinblastine, vincristine, vindesine, vinflunine, vinorelbine, vismodegib, vorinostat, vorozole, yttrium-90 glass microspheres, zinostatin, zinostatin stimalamer, zoledronic acid, or zorubicin.

Furthermore, the compounds of the present invention can be combined with active ingredients, which are well known for the treatment of cancer-related pain and chronic pain. Such combinations include, but are not limited to step II opiods like codeine phosphate, dextropropoxyphene, dihydro-codeine, Tramadol), step III opiods like morphine, fentanyl, buprenorphine, oxymorphone, oxycodone and hydromorphone; and other medications used for the treatment of cancer pain like steroids as Dexamethasone and methylprednisolone; bisphosphonates like Etidronate, Clodronate, Alendronate, Risedronate, and Zoledronate; tricyclic antidepressants like Amitriptyline, Clomipramine, Desipramine, Imipramine and Doxepin; class I antiarrhythmics like mexiletine and lidocaine; anticonvulsants like carbamazepine, Gabapentin, oxcarbazepine, phenytoin, pregabalin, topiramate, alprazolam, diazepam, flurazepam, pentobarbital and phenobarbital.

In addition to those mentioned above, the inventive Bradykinin B1 inhibitors can also be combined with any of the following active ingredients:

active ingredients for Alzheimer's therapy, for example acetylcholinesterase inhibitors (e.g. donepezil, rivastigmine, galantamine, tacrine), NMDA (N-methyl-D-aspartate) receptor antagonists (e.g. memantine); L-DOPA/carbidopa (L-3,4-dihydroxyphenylalanine), COMT (catechol-O-methyltransferase) inhibitors (e.g. entacapone), dopamine agonists (e.g. ropinrole, pramipexole, bromocriptine), MAO-B (monoaminooxidase-B) inhibitors (e.g. selegiline), anticholinergics (e.g. trihexyphenidyl) and NMDA antagonists (e.g. amantadine) for treatment of Parkinson's; beta-interferon (IFN-beta) (e.g. IFN beta-1b, IFN beta-1a Avonex® and Betaferon®), glatiramer acetate, immunoglobulins, natalizumab, fingolimod and immunosuppressants such as mitoxantrone, azathioprine and cyclophosphamide for treatment of multiple sclerosis; substances for treatment of pulmonary disorders, for example beta-2-sympathomimetics (e.g. salbutamol), anticholinergics (e.g. glycopyrronium), methylxanthines (e.g. theophylline), leukotriene receptor antagonists (e.g. montelukast), PDE-4 (phosphodiesterase type 4) inhibitors (e.g. roflumilast), methotrexate, IgE antibodies, azathioprine and cyclophosphamide, cortisol-containing preparations; substances for treatment of osteoarthritis such as non-steroidal anti-inflammatory substances (NSAIDs). In addition to the two therapies mentioned, methotrexate and biologics for B-cell and T-cell therapy (e.g. rituximab, abatacept) should be mentioned for rheumatoid disorders such as rheumatoid arthritis and juvenile idiopathic arthritis. Neurotrophic substances such as acetylcholinesterase inhibitors (e.g. donepezil), MAO (monoaminooxidase) inhibitors (e.g. selegiline), interferons and anticonvulsives (e.g. gabapentin); active ingredients for treatment of cardiovascular disorders such as beta-blockers (e.g. metoprolol), ACE inhibitors (e.g. benazepril), diuretics (e.g. hydrochlorothiazide), calcium channel blockers (e.g. nifedipine), statins (e.g. simvastatin); anti-diabetic drugs, for example metformin and glibenclamide, sulphonylureas (e.g. tolbutamide) and insulin therapy for treatment of diabetes and metabolic syndrome. Active ingredients such as mesalazine, sulfasalazine, azathioprine, 6-mercaptopurine or methotrexate, probiotic bacteria (Mutaflor, VSL#3®, Lactobacillus GG, Lactobacillus plantarum, L. acidophilus, L. casei, Bifidobacterium infantis 35624, Enterococcus fecium SF68, Bifidobacterium longum, Escherichia coli Nissle 1917), antibiotics, for example ciprofloxacin and metronidazole, anti-diarrhoea drugs, for example loperamide, or laxatives (bisacodyl) for treatment of chronic-inflammatory bowel disorders. Immunosuppressants such as glucocorticoids and non-steroidale anti-inflammatory substances (NSAIDs), cortisone, chloroquine, cyclosporine, azathioprine, belimumab, rituximab, cyclophosphamide for treatment of lupus erythematosus. By way of example but not exclusively, calcineurin inhibitors (e.g. tacrolimus and ciclosporin), cell division inhibitors (e.g. azathioprine, mycophenolate mofetil, mycophenolic acid, everolimus or sirolimus), rapamycin, basiliximab, daclizumab, anti-CD3 antibodies, anti-T-lymphocyte globulin/anti-lymphocyte globulin for organ transplants, Vitamin D3 analogues, for example calcipotriol, tacalcitol or calcitriol, salicylic acid, urea, ciclosporine, methotrexate, or efalizumab for dermatological disorders.

Methods of Treating

The present invention relates to a method for using the compounds of the present invention and compositions thereof, to inhibit the Bradykinin B1 receptor.

The present invention relates to a method for using the compounds of the present invention and compositions thereof, to treat mammalian disorders and diseases which include but are not limited to:

Diseases related to pain and inflammation, in particular selected from the group consisting of
visceral pain e.g. related to pancreatitis, interstitial cystitis, renal colic, or prostatitis, chronic pelvic pain, or pain related to infiltrating endometriosis;
neuropathic pain such as post herpetic neuralgia, acute zoster pain, pain related to nerve injury, the dynias, including vulvodynia, phantom limb pain, pain related to root avulsions, pain related to radiculopathy, painful traumatic mononeuropathy, painful entrapment neuropathy, pain related to carpal tunnel syndrome, ulnar neuropathy, pain related to tarsal tunnel syndrome, painful diabetic neuropathy, painful polyneuropathy, trigeminal neuralgia, or pain related to familial amyloid polyneuropathy;
central pain syndromes potentially caused by virtually any lesion at any level of the nervous system including but not limited to pain related to stroke, multiple sclerosis, and spinal cord injury;
postsurgical pain syndromes (including postmastectomy pain syndrome, postthoracotomy pain syndrome, stump pain), bone and joint pain (osteoarthritis), spine pain (including acute and chronic low back pain, neck pain, pain related to spinal stenosis), shoulder pain, repetitive motion pain, dental pain, pain related to sore throat, cancer pain, burn pain including sun-burn, myofascial pain (pain related to muscular injury, fibromyalgia) postoperative, and perioperative pain (including but not limited to general surgery, orthopaedic, and gynaecological surgery); and
acute and chronic pain, chronic pelvic pain, endometriosis associated pain, dysmenorrhea associated pain (primary and secondary), pain associated with uterine fibroids, vulvodynia associated pain, as well as pain associated with angina, or inflammatory pain of varied origins (including but not limited to pain associated with osteoarthritis, rheumatoid arthritis, rheumatic disease, tenosynovitis, gout, ankylosing spondylitis, and bursitis);
and diseases like or related to a disease selected from related to the group consisting of:
gynaecological disorders and/or diseases, or effects and/or symptoms which negatively influence women health including endometriosis, uterine fibroids, pre-eclampsia, hormonal deficiency, spasms of the uterus, or heavy menstrual bleeding;
the respiratory or excretion system including any of inflammatory hyperreactive airways, inflammatory events associated with airways disease like chronic obstructive pulmonary disease, asthma including allergic asthma (atopic or non-atopic) as well as exercise-induced bronchoconstriction, occupational asthma, viral or bacterial exacerbation of asthma, other non-allergic asthmas and wheezy-infant syndrome, chronic obstructive pulmonary disease including emphysema, adult respiratory distress syndrome, bronchitis, pneumonia, cough, lung injury, lung fibrosis, allergic rhinitis (seasonal and perennial), vasomotor rhinitis, angioedema (including hereditary angioedema and drug-induced angioedema including that caused by angiotensin converting enzyme (ACE) or ACE/neutral endopeptidase inhibitors like omepatrilat), pneumoconiosis, including aluminosis, anthracosis, asbestosis, chalicosis, ptilosis, siderosis, silicosis, tabacosis and byssinosis, bowel disease including Crohn's disease and ulcerative colitis, irritable bowel syndrome, pancreatitis, nephritis, cystitis (interstitial cystitis), kidney fibrosis, kidney failure, hyperactive bladder, and overactive bladder;
dermatology including pruritus, itch, inflammatory skin disorders including psoriasis, eczema, and atopic dermatitis;
affection of the joints or bones including rheumatoid arthritis, gout, osteoporosis, osteoarthritis, and ankylosing spondylitis;
affection of the central and peripheral nervous system including neurodegenerative diseases including Parkinson's and Alzheimer's disease, amyotrophic lateral sclerosis (ALS), epilepsy, dementia, headache including cluster headache, migraine including prophylactic and acute use, stroke, closed head trauma, and multiple sclerosis;
infection including HIV infection, and tuberculosis;
trauma associated with oedema including cerebral oedema, burns, sunburns, and sprains or fracture;
poisoning including aluminosis, anthracosis, asbestosis, chalicosis, ptilosis, siderosis, silicosis, tabacosis, and byssinosis uveitis;
diabetes cluster or metabolism like diabetes type 1, diabetes type 2, diabetic vasculopathy, diabetic neuropathy, diabetic retinopathy, post capillary resistance or diabetic symptoms associated with insulitis (e.g. hyperglycaemia, diuresis, proteinuria and increased nitrite and kallikrein urinary excretion), diabetic macular oedema, metabolic syndrome, insulin resistance, obesity, or fat or muscle metabolism;
cachexia associated with or induced by any of cancer, AIDS, coeliac disease, chronic obstructive pulmonary disease, multiple sclerosis, rheumatoid arthritis, congestive heart failure, tuberculosis, familial amyloid polyneuropathy, mercury poisoning (acrodynia), and hormonal deficiency;
cardio-vascular system including congestive heart failure, atherosclerosis, congestive heart failure, myocardial infarct, and heart fibrosis; and
other conditions including septic shock, sepsis, muscle atrophy, spasms of the gastrointestinal tract, benign prostatic hyperplasia, and liver diseases such as non-alcoholic and alcoholic fatty liver disease, non-alcoholic and alcoholic steatohepatitis, liver fibrosis, or liver cirrhosis.

A preferred embodiment of the present invention relates to a method for using the compounds of the present invention and compositions thereof, to treat a gynaecological disease, preferably dysmenorrhea, dyspareunia or endometriosis, endometriosis-associated pain, or other endometriosis-associated symptoms, wherein said symptoms include dysmenorrhea, dyspareunia, dysuria, or dyschezia. Additionally the present invention relates to a method for using the compounds of the present invention and compositions thereof, to treat osteoarthritis, rheumatoid arthritis, gout, neuropathic pain, asthma, cough, lung injury, lung fibrosis, pneumonia, kidney fibrosis, kidney failure pruritus, irritable bowel disease, overactive urinary bladder, diabetes type 1, diabetes type 2, diabetic neuropathy, diabetic retinopathy, diabetic macular oedema, metabolic syndrome, obesity, heart fibrosis, cachexia, muscle atrophy, Alzheimer's disease, and interstitial cystitis.

These disorders have been well characterized in humans, but also exist with a similar etiology in other mammals, and can be treated by administering pharmaceutical compositions of the present invention.

The term “treating” or “treatment” as stated throughout this document is used conventionally, e.g., the management or care of a subject for the purpose of combating, alleviating, reducing, relieving, improving the condition of, etc., of a disease or disorder, such as a gynaecological disease.

Dose and Administration

Based upon standard laboratory techniques known to evaluate compounds useful for the treatment of disorders and/or diseases which are mediated by Bradykinin B1 receptor, by standard toxicity tests and by standard pharmacological assays for the determination of treatment of the conditions identified above in mammals, and by comparison of these results with the results of known medicaments that are used to treat these conditions, the effective dosage of the compounds of this invention can readily be determined for treatment of each desired indication. The amount of the active ingredient to be administered in the treatment of one of these conditions can vary widely according to such considerations as the particular compound and dosage unit employed, the mode of administration, the period of treatment, the age and sex of the patient treated, and the nature and extent of the condition treated.

The total amount of the active ingredient to be administered will generally range from about 0.001 mg/kg to about 200 mg/kg body weight per day, preferably from about 0.01 mg/kg to about 20 mg/kg body weight per day. A preferred administration of the compound of the present invention includes but is not limited to 0.1 mg/kg to about 10 mg/kg body weight per day. Clinically useful dosing schedules will range from one to three times a day dosing to once every four weeks dosing. In addition, “drug holidays” in which a patient is not dosed with a drug for a certain period of time, may be beneficial to the overall balance between pharmacological effect and tolerability. A unit dosage may contain from about 0.5 mg to about 1500 mg of active ingredient, and can be administered one or more times per day or less than once a day. A preferred oral unit dosage for administration of the compounds of the present invention includes but is not limited to 0.1 mg/kg to about 10 mg/kg body weight one to three times a day to once a week. The average daily dosage for administration by injection, including intravenous, intramuscular, subcutaneous and parenteral injections, and use of infusion techniques will preferably be from 0.01 to 200 mg/kg of total body weight. The average daily rectal dosage regimen will preferably be from 0.01 to 200 mg/kg of total body weight. The average daily vaginal dosage regimen will preferably be from 0.01 to 200 mg/kg of total body weight. The average daily topical dosage regimen will preferably be from 0.1 to 200 mg administered between one to four times daily. The transdermal concentration will preferably be that required to maintain a daily dose of from 0.01 to 200 mg/kg of total body weight. The average daily inhalation dosage regimen will preferably be from 0.01 to 100 mg/kg of total body weight.

Of course the specific initial and continuing dosage regimen for each patient will vary according to the nature and severity of the condition as determined by the attending diagnostician, the activity of the specific compound employed, the age and general condition of the patient, time of administration, route of administration, rate of excretion of the drug, drug combinations, and the like. The desired mode of treatment and number of doses of a compound of the present invention or a pharmaceutically acceptable salt or ester or composition thereof can be ascertained by those skilled in the art using conventional treatment tests.

Preferably, the diseases treated with said method are gynaecological disorders, more preferably dysmenorrhea, dyspareunia or endometriosis, endometriosis-associated pain, or other endometriosis-associated symptoms, wherein said symptoms include dysmenorrhea, dyspareunia, dysuria, or dyschezia. Further diseases which can be treated with said method are osteoarthritis, rheumatoid arthritis, gout, neuropathic pain, asthma, cough, lung injury, lung fibrosis, pneumonia, kidney fibrosis, kidney failure pruritus, irritable bowel disease, overactive urinary bladder, diabetes type 1, diabetes type 2, diabetic neuropathy, diabetic retinopathy, diabetic macular oedema, metabolic syndrome, obesity, heart fibrosis, cachexia, muscle atrophy, Alzheimer's disease, and interstitial cystitis.

Preferably, the method of treating the diseases mentioned above is not limited to the treatment of said disease but also includes the treatment of pain related to or associated with said diseases.

The compounds of the present invention can be used in particular in therapy and prevention, i.e. prophylaxis, of genitourinary, gastrointestinal, respiratory or pain-related disease, condition or disorder.

Methods of testing for a particular pharmacological or pharmaceutical property are well known to persons skilled in the art.

The example testing experiments described herein serve to illustrate the present invention and the invention is not limited to the examples given.

It is to be understood that the present invention relates also to any combination of the preferred embodiments described above.

Synthesis of Compounds of General Formula (I) of the Present Invention

Compounds of general formula (I) with the meaning of R¹, R², R³, R⁵, R⁶, R⁷ and R⁸ as defined in general formula (I), can be synthesised according to various general procedures.

Scheme 1 depicts the synthesis starting from synthons of the formula (II), wherein Hal stands for Cl, Br or I, Br being preferred; and wherein ALK stands for C₁-C₅-alkyl, methyl, ethyl and propyl being preferred. The aryl halides of the general formula (II) can be cross-coupled with boronic acids of the general formula (III) or alternatively with their respective pinacol esters to yield compounds of general formula (IV) by Pd-mediated reactions (Suzuki coupling) known to those skilled in the art. A suitable solvent (for example N,N-dimethylformamide, tetrahydrofuran, 1,4-dioxane, 1,2-dimethoxyethane and optionally water) is used and a base (such as triethylamine, potassium carbonate, caesium carbonate) and a catalyst-ligand mixture, for example of palladium(II) acetate/triphenylphosphine, tetrakis(triphenylphosphine)palladium(0), bis(triphenylphosphine)palladium(II) dichloride, bis(diphenylphosphino)ferrocenedichloropalladium (II) is utilised at temperatures between 20° C. and 120° C., preferred at 100° C. Aromatic amines of general formula (IV) may react with cis-, trans- or cis-/trans-mixtures of carboxylic acids of general formula (V) by methods known to those skilled in the art to give the amide compounds of general formula (VI). The reaction is mediated by activating a carboxylic acid of general formula (V) with reagents such as dicyclohexylcarbodiimide (DCC), 1-(3-dimethylaminopropyl)-3-ethylcarbodiimide (EDCI), N-hydroxybenzotriazole (HOBT), N-[(dimethylamino)-(3H-[1,2,3]triazolo[4,5-b]pyridin-3-yloxy)methy den]-N-methylmethanaminium hexafluorophosphate (HATU) or propylphosphonic anhydride (T3P). For example, the reaction with HATU takes place in an inert solvent, such as N,N-dimethylformamide, dichloromethane or dimethyl sulfoxide in the presence of the appropriate aniline general formula (IV) and a tertiary amine (such as triethylamine or diisopropylethylamine) at temperatures between −30° C. and +60° C.

It is also possible to convert a carboxylic acid of the general formula (V) into the corresponding carboxylic acid chloride with an inorganic acid chloride (such as phosphorus pentachloride, phosphorus trichloride or thionyl chloride) and then into the amide compounds of general formula (VI), in pyridine or a solvent (such as dichloromethane, or N,N-dimethylformamide), in the presence of the appropriate amine formula (IV) and a tertiary amine (for example triethylamine) at temperatures between −30° C. and +60° C. The ester moiety of compounds of general formula (VI) are then converted to the final target compounds of general formula (I) by ester group saponification in a solvent (such as tetrahydrofuran, methanol or N,N-dimethylformamide) using an appropriate base (for example aqueous lithium hydroxide or aqueous sodium hydroxide) at temperatures between 0° C. and +80° C. Alternatively, the ester compounds of general formula (VI) can be converted to the final target compounds of general formula (I) by ester group saponification using an appropriate inorganic acid (for example hydrochloric acid or sulfuric acid) at temperatures between 0° C. and +80° C., usually at circa +60° C.

Aryl halides of the general formula (II) are either commercially available or can be synthesised by those skilled in the art from the corresponding carboxylic acid compound. For example by reacting the corresponding carboxylic acid with an alcohol (such as methanol, ethanol or propanol) in inorganic acid (for example hydrochloric acid or sulfuric acid) at temperatures between 0° C. and 100° C.

The starting materials of the general formula (II) are either commercially available or can be synthesized via methods known to those skilled in the art from appropriate precursors. For example, the amino group may be obtained by reduction of the corresponding nitro group with hydrogen in the presence of a palladium catalyst in solvents like ethanol, ethyl acetate or mixtures thereof. Alternatively, the nitro group may be reduced using iron powder in solvents like methanol or ethanol in the presence of acid (such as hydrochloric or acetic acid). The nitro group may be introduced by classical methods like treatment with nitric acid/sulphuric acid or potassium nitrate/sulphuric acid (with appropriate concentration and volume ratio) at temperatures between 0° C. and 25° C. The sequence of reaction steps (nitro reduction, Suzuki reaction, amide formation, nitrile hydrolysis) may be changed as appropriate.

The carboxylic acids of the general formula (V) are either commercially available or can be synthesized via methods known to those skilled in the art from appropriate precursors. For example, arylcyclopropanecarboxylic acids may be prepared from the corresponding arylacetonitrile by cyclopropanation with 1-bromo-2-chloroethane (1.5 eq) in aqueous base (such as sodium hydroxide solution) in the presence of benzyltriethylammonium chloride (0.02 eq.) and subsequent acidic or basic hydrolysis of the nitrile with e.g. lithium hydroxide in water or concentrated hydrochloric acid at temperatures between 20° C. and 100° C.

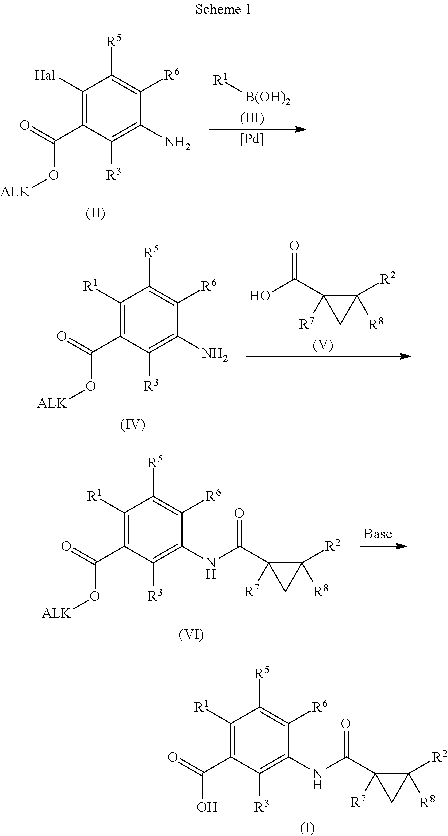

Scheme 2 depicts the synthesis starting from synthons of the formula (VII), wherein Hal stands for Cl, Br or I, Br being preferred. The aryl halides of the general formula (VII) can be cross-coupled with boronic acids of the general formula (III) or alternatively with their respective pinacol esters to yield compounds of general formula (VIII) by Pd-mediated reactions (Suzuki coupling) known to those skilled in the art. A suitable solvent (for example N,N-dimethylformamide, tetrahydrofuran, 1,4-dioxane, 1,2-dimethoxyethane and optionally water) is used and a base (such as triethylamine, potassium carbonate, or caesium carbonate) and a catalyst-ligand mixture, for example of palladium(II) acetate/triphenylphosphine, tetrakis(triphenylphosphine)palladium(0), bis(triphenylphosphine)-palladium(II) dichloride, bis(diphenylphosphino)ferrocenedichloropalladium (II) is utilised at temperatures between 20° C. and 120° C., preferred at 100° C. The nitro group of a compound of general formula (VIII) is then reduced to the corresponding aniline of general formula (IX) by reaction under a hydrogen atmosphere in the presence of a palladium catalyst (for example 5-10% palladium on carbon) in an appropriate solvent (for example ethanol or ethyl acetate) at temperatures between 0° C. and 100° C. In analogy to the procedures described for Scheme 1, amide coupling gives compounds of the general formula (X). The ester moiety of a compound of general formula (X) is hydrolysed to carboxylic acid of general formula (I) by reaction with either an inorganic base (for example aqueous lithium hydroxide or aqueous sodium hydroxide) or an inorganic acid (for example hydrochloric acid or sulfuric acid) optionally in an inert solvent (such as tetrahydrofuran, 1,4-dioxane or N,N-dimethylformamide) at temperatures between 10° C. and 100° C.

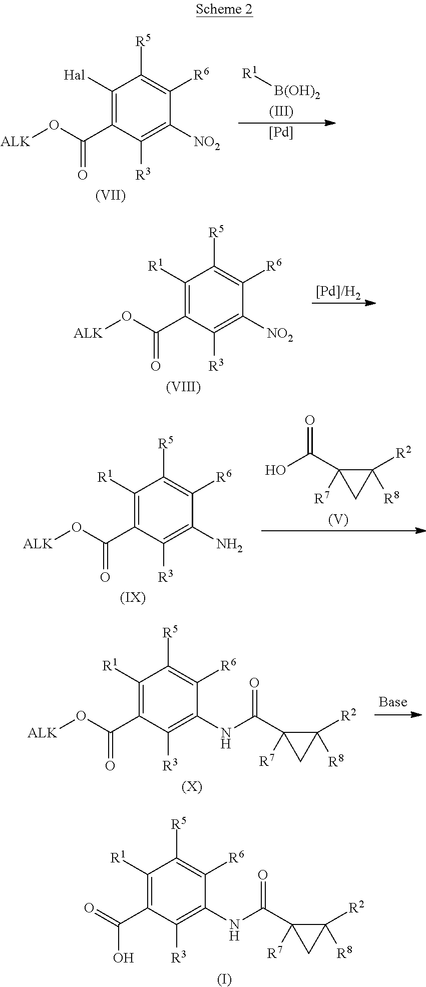

Scheme 3 shows an alternative approach to synthesis compounds of general formula (I) where R¹ is an N-linked optionally substituted 5-membered heteroaryl group, for example pyrazolyl or imidazolyl, or alternatively R¹ is an N-linked optionally substituted bicyclic 8- to 10-membered heteroaryl group, for example indole. Starting from synthons of the general formula (VII) (wherein Hal stands for F, Cl or Br) the aryl halide can first be substituted by a nucleophile of general formula (XXIV) to yield a compound of general formula (VIII). The substitution takes place in a dipolar aprotic solvent such as acetonitrile, dimethylsulfoxide or N,N-dimethylformamide and in the presence of an appropriate base (for example potassium carbonate) at temperatures between 20° C. and 100° C., preferably at 60° C. In analogy to the procedures described for Scheme 2, nitro group reduction followed by amide formation gives compounds of general formula (X) that are subsequently converted to the final targets of general formula (I) by ester group hydrolysis.

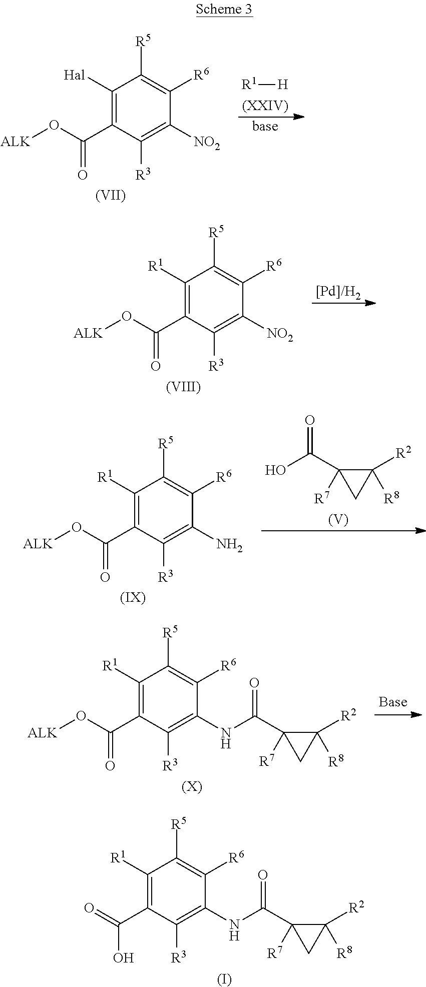

In Scheme 3 general formula (XXIV) represents R¹-H wherein R¹ is an N-linked optionally substituted 5-membered heteroaryl group, for example pyrazolyl or imidazolyl, or alternatively R¹ is an N-linked optionally substituted bicyclic 8- to 10-membered heteroaryl group.

Scheme 4 shows an alternative approach in which the sequence of reaction steps is changed and the nitrile moiety of general formula (IX) is transformed into an ester group in two steps (wherein ALK stands for a C₁-C₅-alkyl, such as methyl, ethyl or propyl) and later revealed as the carboxylic acid group. Starting from synthons of general formula (IX), first the nitrile is converted in to the carboxylic acid of general formula (XI) and then reacted with a suitable alcohol (such as methanol, ethanol or propanol) in inorganic acid (for example hydrochloric acid or sulfuric acid) at temperatures between 0° C. and 100° C. to form ester compounds of general formula (XII). In analogy to the procedures described for Scheme 1, amide coupling gives compounds of the general formula (XIII), followed by ester group saponification to yield the target compounds of general formula (I).

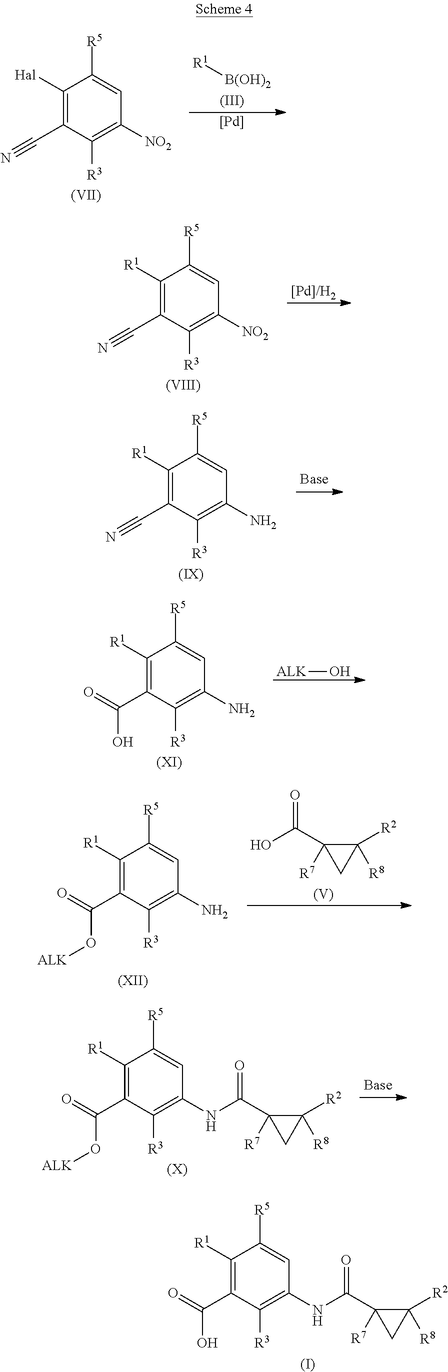

Scheme 5 shows an alternative approach to synthesize compounds of general formula (I) where R¹ is an N-linked optionally substituted 5-membered heteroaryl group, for example pyrazolyl, or imidazolyl. Starting from synthons of the general formula (II) (wherein Hal stands for F, Cl or Br; and wherein ALK stands C₁-C₅-alkyl, such as for methyl, ethyl or propyl) the aryl halide can first be substituted by a nucleophile of general formula (XXIV) to yield a compound of general formula (IV). In analogy to the procedures described for Scheme 1, amide coupling gives compounds of the general formula (VI), followed by ester group saponification to yield the target compounds of general formula (I).

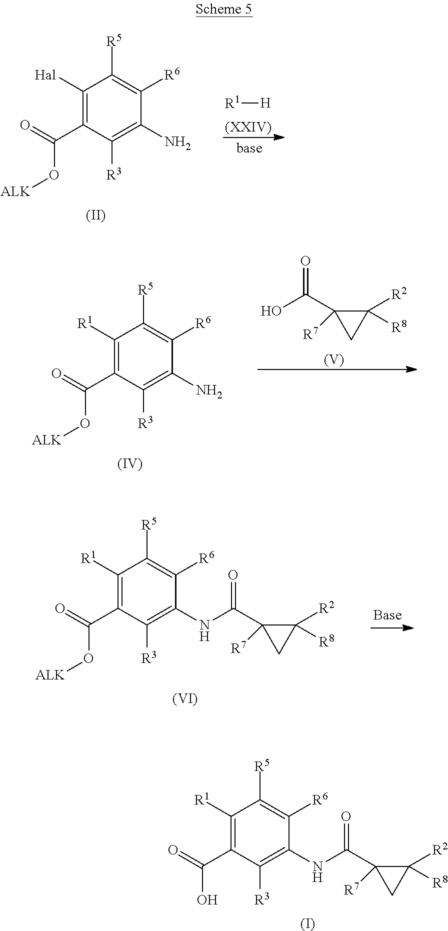

In Scheme 5 general formula (XXIV) represents R¹-H wherein R¹ is a 5-membered heteroaryl linked through a nitrogen atom.

Scheme 6 depicts the synthesis starting from synthons of the formula (XXV), wherein Hal stands for Br or I, Br being preferred. The aryl halides of the general formula (XXV) can be cross-coupled with boronic acids of the general formula (III) or alternatively with their respective pinacol esters to yield compounds of general formula (XXVI) by Pd-mediated reactions (Suzuki coupling) known to those skilled in the art. A suitable solvent (for example N,N-dimethylformamide, tetrahydrofuran, 1,4-dioxane, 1,2-dimethoxyethane and optionally water) is used and a base (such as triethylamine, potassium carbonate, caesium carbonate) and a catalyst-ligand mixture, for example of palladium(II) acetate/triphenylphosphine, tetrakis(triphenylphosphine)palladium(0), bis(triphenylphosphine)-palladium(II) dichloride, bis(diphenylphosphino)ferrocenedichloropalladium (II) is utilised at temperatures between 20° C. and 120° C., preferred at 100° C. The chloro group of a compound of general formula (XXVI) is converted to a nitrile group to yield compounds of general formula (IX), by Pd-mediated cyanation reactions with potassium ferrocyanide known to those skilled in the art. A suitable solvent mixture (for example 1,4-dioxane or tetrahydrofuran and optionally water) is used and a base (such as potassium acetate) and a catalyst-ligand mixture (for example tris [dibenzylideneacetone]dipalladium/dicyclohexyl [2′,4′,6′-tri(propan-2-yl)biphenyl-2-yl]phosphane) is utilised at temperatures between 20° C. and 120° C., usually 100° C. In analogy to the procedures described for Scheme 4, the nitrile moiety of general formula (IX) is converted in to the carboxylic acid of general formula (XI) and then reacted with a suitable alcohol (such as methanol, ethanol or propanol; wherein ALK stands for C₁-C₅-alky, such as methyl, ethyl or propyl) in inorganic acid (for example hydrochloric acid or sulfuric acid) at temperatures between 0° C. and 100° C. to form ester compounds of general formula (XII). In analogy to the procedures described for Scheme 1, amide coupling gives compounds of the general formula (XIII), followed by ester group saponification to yield the target compounds of general formula (I). The sequence of reaction steps (nitrile hydrolysis, amide formation) may be changed as appropriate. In analogy with Scheme 4, final compounds of general formula (I) can be accessed directly via nitrile group hydrolysis carried out as a final transformation.

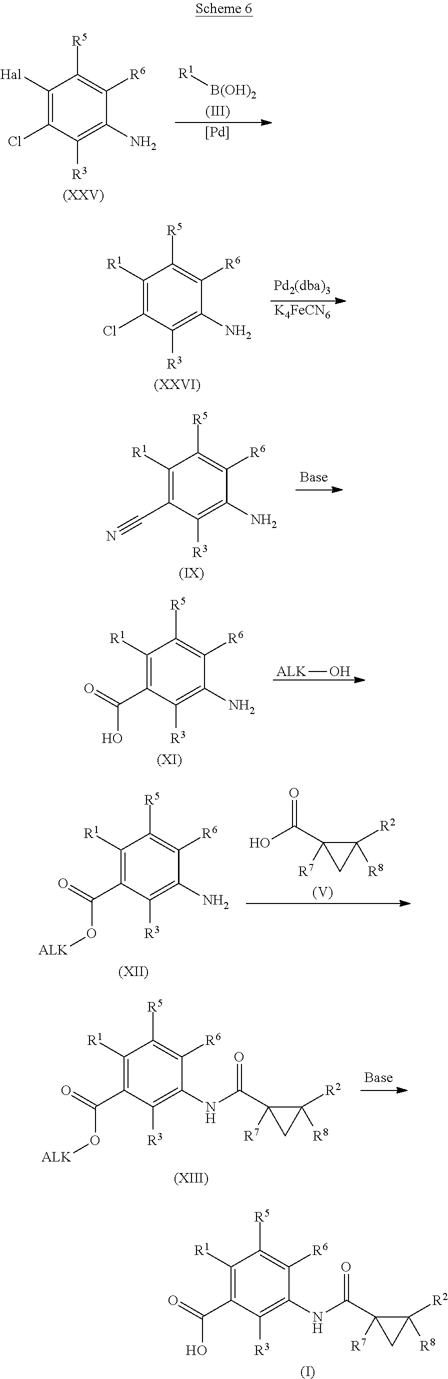

### EXPERIMENTAL SECTION

The example testing experiments described herein serve to illustrate the present invention and the invention is not limited to the examples given.

The following table lists the abbreviations used in this paragraph, and in the examples section.

| Abbreviation | Meaning |
|---|---|
| BSA | Bovine Serum Albumin |
| Cₛ₂CO₃ | Cesium carbonate |
| Cu(I)Cl | Copper(I) chloride |
| ca. | circa |
| DCE | 1,2-Dichloroethane |
| DCM | Dichloromethane |
| DIAD | Diisopropyl azodicarboxylate |
| DIPEA | N-Ethyl-N-isopropylpropan-2-amine |
| DMAP | N,N-Dimethylpyridin-4-amine |
| DME | 1,2-Dimethoxyethane |
| DMF | N,N-Dimethylformamide |
| DMSO | Dimethyl sulfoxide |
| DP | Desired product |
| EE | Ethyl acetate |
| h | Hour |
| HATU | N-[(Dimethylamino)(3H[1,2,3]triazolo[4,5-b]pyridin-3- |
| | yloxy)methylene]-N-methylmethanaminium hexafluorophosphate |
| HBr | Hydrogen bromide |
| HCl | Hydrochloric acid |
| hex | n-Hexane |
| HPLC | high performance liquid chromatography |
| HNO₃ | Nitric acid |
| H₂SO₄ | Sulfuric acid |
| Int | Intermediate |
| IPC | In process check |
| K₂CO₃ | Potassium carbonate |
| LC-MS | liquid chromatography-mass spectrometry |
| LCMS | liquid chromatography-mass spectrometry |
| LiOH | Lithium hydroxide |
| M | Molar |
| μW | Microwave |
| MeCN | Acetonitrile |
| MeOH | Methanol |
| MgSO₄ | Magnesium sulfate |
| min | Minute(s) |
| N | Normal |
| Na₂CO₃ | Sodium carbonate |
| NaH | Sodium hydride |
| NaHCO₃ | Sodium bicarbonate |
| NaI | Sodium iodide |
| NaOH | Sodium hydroxide |
| Na₂SO₄ | Sodium sulfate |
| NH₄Cl | Ammonium chloride |
| NMR | nuclear magnetic resonance spectroscopy |
| PdCl₂(PPh₃)₂ | Bis(triphenylphosphine)palladium(II) dichloride |
| Pd(dppf)Cl₂ | [1,1′-Bis(diphenylphosphino)ferrocene]dichloropalladium(II) |
| Pd(dppf)Cl₂• | 1,1′-Bis(diphenylphosphino)ferrocene-palladium(II)dichloride |
| CH₂Cl₂ | dichloromethane complex |
| PPh₃ | Triphenylphosphine |
| ppm | parts per million |
| Py | Pyridine |
| RT | Room temperature |
| rt | Retention time |
| Rt | Retention time |
| sat. | Saturated |
| SEM | 2-(trimethylsilyl)ethoxymethyl |
| SM | Starting material |
| STAB | Sodium triacetoxyborohydride |
| HSnBu₃ | Tributyltin hydride |
| TMS-azide | Azidotrimethylsilane |
| TMS-N₃ | Azidotrimethylsilane |
| T3P | Propylphosphonic anhydride |
| TBAB | Tetra-N-butylammonium bromide |
| TBAI | Tetra-N-butylammonium iodide |
| TBME | tert-Butyl methyl ether |
| TEA | Triethylamine |
| TFA | Trifluoroacetic acid |
| (Tf)₂O | Trifluoromethanesulfonic anhydride |
| TfO— | Trifluoromethanesulfonate |
| THF | Tetrahydrofuran |

Analysis Methods

Analytical LCMS Methods

Method 1: Instrument: Waters Acquity Platform ZQ4000; column: Waters BEHC 18, 50 mm×2.1 mm, 1.7 μm; eluent A: water/0.05% formic acid, eluent B: acetonitrile/0.05% formic acid; gradient: 0.0 min 98% A→0.2 min: 98% A→1.7 min: 10% A→1.9 min: 10% A→2 min: 98% A→2.5 min: 98% A; flow: 1.3 ml/min; column temperature: 60° C.; UV-detection: 200-400 nm.

Method 2: Instrument: Waters Acquity LCT; column: Phenomenex Kinetex C18, 50 mm×2.1 mm, 2.6 μm; eluent A: water/0.05% formic acid, eluent B: acetonitrile/0.05% formic acid; gradient: 0.0 min 98% A→0.2 min: 98% A→1.7 min: 10% A→1.9 min: 10% A→2 min: 98% A→2.5 min: 98% A; flow: 1.3 ml/min; column temperature: 60° C.; UV-detection: 200-400 nm.

Method 3: Instrument: Waters Acquity UPLCMS SingleQuad; Column: Acquity UPLC BEH C18 1.7 μm, 50×2.1 mm; eluent A: water+0.1 vol % formic acid (99%), eluent B: acetonitrile; gradient: 0-1.6 min 1-99% B, 1.6-2.0 min 99% B; flow 0.8 ml/min; temperature: 60° C.; DAD scan: 210-400 nm.

Method 4: Instrument: Waters Acquity UPLCMS SingleQuad; Column: Acquity UPLC BEH C18 1.7 μm, 50×2.1 mm; eluent A: water+0.2 vol % aqueous ammonia (32%), eluent B: acetonitrile; gradient: 0-1.6 min 1-99% B, 1.6-2.0 min 99% B; flow 0.8 ml/min; temperature: 60° C.; DAD scan: 210-400 nm.

Method 5: Instrument MS: Waters ZQ; instrument HPLC: Waters UPLC Acquity; column: Acquity BEH C18 (Waters), 50 mm×2.1 mm, 1.7 μm; eluent A: water+0.1% formic acid, eluent B: acetonitrile (Lichrosolv Merck); gradient: 0.0 min 99% A—1.6 min 1% A—1.8 min 1% A—1.81 min 99% A—2.0 min 99% A; oven: 60° C.; flow: 0.800 ml/min; UV-detection PDA 210-400 nm.

LC-MS, Analytical Method A: Routine High Throughput Analysis

Column: Kinetex Core-Shell C18, 2.1×50 mm, 5 μm; Eluent A: Water+0.1% Formic acid, Eluent B: Acetonitrile+0.1% Formic acid; Gradient 0.00 mins 95% A→1.20 mins 100% B→1.30 mins 100% B→1.31 mins 95% A; column temperature: 40° C.; flow rate 1.2 ml/min; injection volume: 3 μl; UV-detection range: 210-420 nm.

LC-MS, Analytical Method B: Routine High Throughput Analysis

Column: Waters Atlantis dC18, 2.1×50 mm, 3 μm; Eluent A: Water+0.1% Formic acid, Eluent B: Acetonitrile+0.1% Formic acid; Gradient 0.00 mins 95% A→2.5 mins 100% B→2.7 mins 100% B→2.71 mins 5% A→3.5 mins 5% A; column temperature: 40° C.; flow rate 1.0 ml/min; injection volume: 3 μl; UV-detection range: 210-420 nm.

LC-MS, Analytical Method C: Routine High Throughput Analysis at High pH

Column: Phenomenex Gemini-NX C18, 2.0×50 mm, 3 μm; Eluent A: 2 mM ammonium bicarbonate, buffered to pH10, Eluent B: Acetonitrile; Gradient 0.00 mins 99% A→1.80 mins 100% B→2.10 mins 100% B→2.30 mins 99% A→3.50 mins 99% A; column temperature: 40° C.; flow rate 1.0 ml/min; injection volume: 3 μl; UV-detection range: 210-420 nm.

LC-MS, Analytical Method D:

Column: Waters Atlantis dC18, 2.1×100 mm, 3 μm; Eluent A: Water+0.1% Formic acid, Eluent B: Acetonitrile+0.1% Formic acid; Gradient 0.00 mins 95% A→5.00 mins 100% B→5.40 mins 100% B→5.42 mins 95% A→7.00 mins 95% A; column temperature: 40° C.; flow rate 0.6 ml/min; injection volume: 3 μl; UV-detection range: 210-420 nm.

LC-MS, Analytical Method E: High pH

Column: Phenomenex Gemini-NX C18, 2.0×100 mm, 3 μm; Eluent A: 2 mM ammonium bicarbonate, buffered to pH10, Eluent B: Acetonitrile; Gradient 0.00 mins 95% A→5.50 mins 100% B→5.90 mins 100% B→5.92 mins 95% A→7.00 mins 95% A; column temperature: 40° C.; flow rate 0.5 ml/min; injection volume: 3 μl; UV-detection range: 210-420 nm.

LC-MS, Analytical Method F:

Column: Phenomenex Kinetix-XB C18, 2.1×100 mm, 1.7 μm; Eluent A: Water+0.1% Formic acid, Eluent B: Acetonitrile+0.1% Formic acid; Gradient 0.00 mins 95% A→5.30 mins 100% B→5.80 mins 100% B→5.82 mins 95% A→7.00 mins 95% A; column temperature: 40° C.; flow rate 0.6 ml/min; injection volume: 1 μl; UV-detection range: 200-400 nm.

Purification Methods:

Biotage Isolera™ chromatography system using pre-packed silica and pre-packed modified silica cartridges.

Preparative HPLC, Method A: High pH

Column: Waters Xbridge C18, 30×100 mm, 10 μm; Solvent A: Water+0.2% Ammonium hydroxide, Solvent B: Acetonitrile+0.2% Ammonium hydroxide; Gradient 0.00 mins 90% A→0.55 mins 90% A→14.44 mins 95% B→16.55 mins 95% B→16.75 90% A; column temperature: room temperature; flow rate 40 ml/min; injection volume: 1500 μl; Detection: UV 215 nm.

Preparative HPLC, Method B: Low pH

Column: Waters Sunfire C18, 30×100 mm, 10 μm; Solvent A: Water+0.1% Formic acid, Solvent B: Acetonitrile+0.1% Formic acid; Gradient 0.00 mins 90% A→0.55 mins 90% A→14.44 mins 95% B→16.55 mins 95% B→16.75 90% A; column temperature: room temperature; flow rate 40 ml/min; injection volume: 1500 μl; Detection: UV 215 nm.

Preparative HPLC Methods

Preparative HPLC, Method 1:

System: Waters autopurification system: Pump 2545, Sample Manager 2767, CFO, DAD 2996, ELSD 2424, SQD; Column: XBrigde C18 5 μm 100×30 mm; Solvent: A=H2O+0.1% Vol. formic acid (99%), B=acetonitrile; Gradient: 0-8 min 10-100% B, 8-10 min 100% B; Flow: 50 mL/min; temperature: room temp.; Solution: Max. 250 mg/max. 2.5 mL DMSO or DMF; Injection: 1×2.5 mL; Detection: DAD scan range 210-400 nm; MS ESI+, ESI−, scan range 160-1000 m/z.

Preparative HPLC, Method 2:

System: Waters autopurification system: Pump 2545, Sample Manager 2767, CFO, DAD 2996, ELSD 2424, SQD; Column: XBrigde C18 5 μm 100×30 mm; Solvent: A=H2O+0.1% Vol. ammonia (99%), B=acetonitrile; Gradient: 0-8 min 10-100% B, 8-10 min 100% B; Flow: 50 mL/min; temperature: room temp.; Solution: Max. 250 mg/max. 2.5 mL DMSO or DMF; Injection: 1×2.5 mL; Detection: DAD scan range 210-400 nm; MS ESI+, ESI−, scan range 160-1000 m/z.

### EXAMPLES

Chemical Naming of the Examples and Intermediates was Performed Using ACD Software by ACD/LABS or Marvin Software by ChemAxon.

Reaction times are either specified explicitly in the protocols of the experimental section, or reactions were run until completion. Chemical reactions were monitored and their completion was judged using methods well known to the person skilled in the art, such as thin layer chromatography, e.g. on plates coated with silica gel, or by LCMS methods.

### Intermediate 1A: Methyl 5-amino-2-bromobenzoate

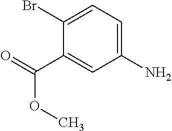

To a 0° C. solution of 5-amino-2-bromobenzoic acid (10 g, 46.3 mmol) in methanol (103 mL) was added thionyl chloride (1.1 eq., 3.7 mL, 50.9 mmol) dropwise. The resulting mixture was stirred at 70° C. for 16 h. The mixture was evaporated to dryness. The resulting grey solid was used without further purification.

LCMS (method 3): Rₜ=0.90 min; MS (ESIPos) m/z=230/232 (M+H)⁺, Br isotope pattern.

### Intermediate 2A: Methyl 5-amino-2-(1-cyclobutyl-1H-pyrazol-4-yl)benzoate

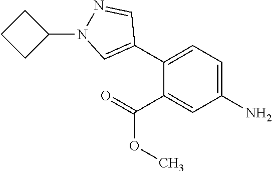 [SMILES: COC(=O)c1cc(N)ccc1-c1cnn(C2CCC2)c1]

Under an atmosphere of nitrogen to a mixture of methyl 5-amino-2-bromobenzoate (Int. 1A, 11.5 g, 50.38 mmol), 1-cyclobutyl-4-(4,4,5,5-tetramethyl-1,3,2-dioxaborolan-2-yl)-1H-pyrazole (15.0 g, 60.45 mmol) and potassium carbonate (22.98 g, 166.24 mmol) in 1,2-dimethoxyethane (183 mL) and water (91 mL) was added Pd(PPh₃)₂Cl₂ (425 mg, 0.61 mmol) and the reaction mixture heated at 90° C. until completion. The reaction was cooled to RT, diluted with water (200 mL) and extracted with ethyl acetate (150 mL). The organic layer was washed with water (100 mL), brine (100 mL), dried (Na₂SO₄), filtered and concentrated at reduced pressure. The residue was purified by Biotage Isolera™ chromatography (using a gradient of eluents; 0-40% EE in heptane) to give the title compound (9.61 g, 70% yield) as a golden oil.

¹H NMR (500 MHz, DMSO-d6) δ [ppm] 7.73 (d, J=0.7 Hz, 1H), 7.37 (d, J=0.6 Hz, 1H), 7.11 (d, J=8.3 Hz, 1H), 6.80 (d, J=2.5 Hz, 1H), 6.70 (dd, J=8.3, 2.5 Hz, 1H), 5.31 (s, 2H), 4.86-4.73 (m, 1H), 3.69 (s, 3H), 2.48-2.31 (m, 4H), 1.83-1.71 (m, 2H).

LCMS (method 3): Rₜ=0.88 min; MS (ESIPos) m/z=272.8 (M+H)⁺.

### Intermediate 3A: 5-Bromo-2-(1,1-difluoroethyl)pyridine

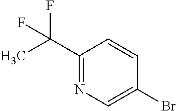

Three pressure tubes were each charged with 1-(5-bromopyridin-2-yl)ethanone (1.0 g, 5.0 mmol) and [bis(2-methoxyethyl)amino]sulfur trifluoride, (2.7M; 50 wt. %) solution in toluene (5.55 mL, 15 mmol). The pressure tubes were sealed and heated at 80° C. for 5 hours. After cooling to RT, the reaction mixtures were combined and diluted with TBME (100 mL), washed with 2M aqueous potassium carbonate solution (2×50 mL) and brine (30 mL), dried (Na₂SO₄) and concentrated at reduced pressure. The residue was purified by Biotage Isolera™ chromatography (using a gradient of eluents; 0-20% TBME in heptane) to afford the title compound (2.4 g, 66% yield) as a pale yellow oil.

¹H NMR (500 MHz, Chloroform-d) δ [ppm] 8.71-8.68 (m, 1H), 7.93 (dd, J=8.4, 2.3 Hz, 1H), 7.55 (dd, J=8.4, 0.6 Hz, 1H), 2.00 (t, J=18.6 Hz, 3H).

LCMS (Analytical Method A): Rₜ=1.25 min; MS (ESIPos) m/z=221.7/223.7 (M+H)⁺, Br isotope pattern.

### Intermediate 4A: 2-(1,1-Difluoroethyl)-5-(4,4,5,5-tetramethyl-1,3,2-dioxaborolan-2-yl)pyridine

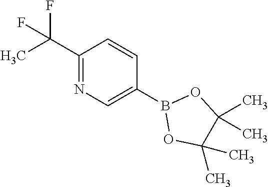 [SMILES: CC(F)(F)c1ccc(B2OC(C)(C)C(C)(C)O2)cn1]

To a solution of 5-bromo-2-(1,1-difluoroethyl)pyridine (2.40 g, 9.836 mmol) and bis(pinacolato)diboron (2.748 g, 10.82 mmol) in 1,4-dioxane (40 mL) was added and potassium acetate (2.896 g, 29.5 mmol) at RT. Nitrogen gas was bubbled through the mixture for 5 min and 1,1′-bis(diphenylphosphino)ferrocenepalladium(II) chloride (125 mg, 0.153 mmol) was then added. The mixture was heated at 100° C. for 2 hours. The reaction mixture was diluted with EE (50 mL), filtered over Celite® and washed with EE (50 mL). The filtrate was concentrated at reduced pressure and the residue was purified by Biotage Isolera™ chromatography (using a gradient of eluents; 0-100% EE in heptane) to afford the title compound (2.24 g, 80% yield) as a white solid.

¹H NMR (500 MHz, Chloroform-d) δ [ppm] 8.96 (br. s, 1H), 8.17 (dd, J=7.8, 1.4 Hz, 1H), 7.62 (d, J=7.8 Hz, 1H), 2.01 (t, J=18.6 Hz, 3H), 1.36 (s, 12H).

LCMS (Analytical Method A): Rₜ=0.81 min; product did not ionise by LCMS.

### Intermediate 5A: 1-(5-Bromopyridin-2-yl)propan-1-one

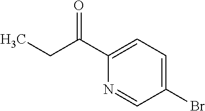

5-Bromopyridine-2-carbonitrile (14.0 g, 76.5 mmol) was dissolved in dry tetrahydrofuran (280 mL) and cooled to −20° C. Ethylmagnesium bromide (31.9 mL of a 3M solution in diethyl ether, 95.6 mmol) was added dropwise at this temperature, with the reaction mixture allowed to warm to RT over 2 hours. After this time 1M aqueous hydrogen chloride solution was then added slowly at 0° C. and the mixture was allowed to re-warm to room temperature and was diluted with ethyl acetate. The organic layer was isolated, washed with saturated aqueous sodium chloride solution, dried (MgSO₄), filtered and concentrated in vacuo, after which time the golden oil crystallised to afford the title compound (15.51 g, 90% yield). No further purification was necessary.

¹H NMR (250 MHz, Chloroform-d) δ [ppm] 8.74 (d, J=1.6 Hz, 1H), 8.02-7.89 (m, 2H), 3.22 (q, J=7.3 Hz, 2H), 1.23 (t, J=7.3 Hz, 3H).

LCMS (Analytical Method A): Rₜ=1.13 min; MS (ESIPos) m/z=213.8/215.8 (M+H)⁺, Br isotope pattern.

### Intermediate 6A: 5-Bromo-2-(1,1-difluoropropyl)pyridine

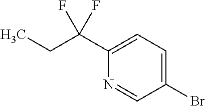

To a solution of 1-(5-bromopyridin-2-yl)propan-1-one (15.51 g, 72.5 mmol) dissolved in 1,2-dichloroethane (181 mL) under nitrogen was added diethylaminosulfur trifluoride (38.29 mL, 289.81 mmol) dropwise giving an orange solution. The reaction was then warmed to 60° C. and stirred at this temperature for 16 hours. After this time, the reaction mixture was cooled to 0° C. and was diluted with 2M aqueous sodium hydroxide solution dropwise (CAUTION: vigourous reaction). The organic layer was removed and washed with saturated aqueous sodium chloride solution, dried (MgSO₄), filtered and concentrated in vacuo. The residue was purified by Biotage Isolera™ chromatography (using a gradient of eluents; 0-20% TBME in heptane). Attempted separation failed, with ˜20% SM remaining. The residual material was dissolved in 1,2-dichloroethane (182 mL) and diethylaminosulfur trifluoride (7.65 mL, 19.6 mmol) dropwise. The reaction was then warmed to 60° C. and stirred at this temperature for 24 hours. After this time, the reaction mixture was cooled to 0° C. and was diluted with 2M aqueous sodium hydroxide solution dropwise (CAUTION: vigourous reaction). The organic layer was removed and washed with saturated aqueous sodium chloride solution, dried (MgSO₄), filtered and concentrated in vacuo. The residue was purified by Biotage Isolera™ chromatography (using a gradient of eluents; 0-20% TBME in heptane) to afford the title compound (11.8 g, 62% yield) as a pale brown oil.

¹H NMR (250 MHz, Chloroform-d) δ [ppm] 8.74 (d, J=2.1 Hz, 1H), 7.95 (dd, J=8.4, 2.3 Hz, 1H), 7.55 (d, J=8.4 Hz, 1H), 2.34 (m, 2H), 1.02 (t, J=7.5 Hz, 3H).

LCMS (Analytical Method A): Rₜ=1.21 min; MS (ESIPos) m/z=235.8/237.8 (M+H)⁺.

### Intermediate 7A: 2-(1,1-Difluoropropyl)-5-(4,4,5,5-tetramethyl-1,3,2-dioxaborolan-2-yl)pyridine

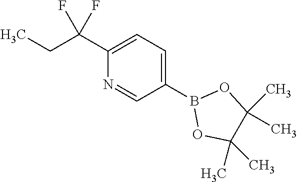 [SMILES: CCC(F)(F)c1ccc(B2OC(C)(C)C(C)(C)O2)cn1]

A solution of 5-bromo-2-(1,1-difluoropropyl)pyridine (Int. 6A, 11.76 g, 44.84 mmol), bis(pinacolato)diboron (12.52 g, 49.32 mmol), potassium acetate (13.20 g, 134.50 mmol) and 1,1′-bis(diphenylphosphino)ferrocenepalladium(II) chloride (820 mg, 1.21 mmol) in 1,4-dioxane (250 mL) was degassed via nitrogen gas balloon for 5 minutes. The nitrogen inlet was removed, a condenser equipped with the reaction mixture heated at 100° C. for 16 hours. After this time, further 1,1′-bis(diphenylphosphino)ferrocenepalladium(II) chloride (820 mg, 1.21 mmol) was added and the solution degassed for 5 minutes. The nitrogen inlet was removed, a condenser equipped with the reaction mixture heated at 100° C. for 6 hours. After this time, the reaction mixture was allowed to cool to RT, diluted with EE, filtered through a plug of Celite® and concentrated in vacuo. The residue was purified by Biotage Isolera™ chromatography (using a gradient of eluents; 0-30% EE in heptane) to afford the title compound as a golden oil that crystallised upon standing (11.25 g, 89% yield).

¹H NMR (250 MHz, Chloroform-d) δ [ppm] 9.00 (br. s, 1H), 8.19 (dd, J=7.8, 1.5 Hz, 1H), 7.62 (dd, J=7.8, 0.8 Hz, 1H), 2.35 (m, 2H), 1.38 (s, 12H), 1.00 (t, J=7.5 Hz, 3H).

LCMS (Analytical Method A): Rₜ=0.89 min; MS (ESIPos) m/z=201.95 (M+H-C₆H₁₂)⁺.

### Intermediate 8A: Methyl 2-(1-cyclobutyl-1H-pyrazol-4-yl)-5-nitrobenzoate

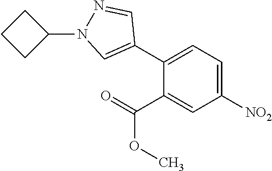 [SMILES: COC(=O)c1cc([N+](=O)[O-])ccc1-c1cnn(C2CCC2)c1]

1-Cyclobutyl-4-(4,4,5,5-tetramethyl-1,3,2-dioxaborolan-2-yl)-1H-pyrazole (1.50 g, 6.0 mmol) and methyl 2-bromo-5-nitrobenzoate (1.66 g, 6.0 mmol) in DME (30 mL) and water (15 mL) was degassed with nitrogen for 5 minutes. Pd(PPh₃)₂Cl₂ (127 mg, 0.18 mmol) and K₂CO₃ (2.5 g, 18.1 mmol) were then added and the reaction was heated to 100° C. for 2 hours. The reaction was then cooled to RT and diluted with water (30 mL) and extracted with EE (50 mL). The organic layer was then washed with brine (2×30 mL), dried (Na₂SO₄), filtered and concentrated at reduced pressure. The residue was purified by Biotage Isolera™ chromatography (using a gradient of eluents; 0-50% EE in heptane) to afford the title compound (1.72 g, 72% yield) as a yellow oil.

¹H NMR (500 MHz, DMSO-d6) δ 8.43 (d, J=2.5 Hz, 1H), 8.33 (dd, J=8.7, 2.6 Hz, 1H), 8.20 (s, 1H), 7.83 (d, J=8.7 Hz, 1H), 7.72 (s, 1H), 4.97-4.81 (m, 1H), 3.85 (s, 3H), 2.45-2.33 (m, 4H), 1.87-1.76 (m, 2H).

LCMS (Analytical Method A): Rₜ=1.19 min; MS (ESIPos) m/z=302 (M+H)⁺.

In analogy to the procedure described for Intermediate 8A, the following intermediates were prepared using methyl 2-bromo-5-nitrobenzoate and the appropriate boronic acids or, respectively, the corresponding pinacol boronic esters as starting materials.

| Int. | Structure | Name | Analytical Data |
|---|---|---|---|
| 9A | 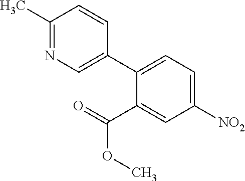 [SMILES: COC(=O)c1cc([N+](=O)[O-])ccc1-c1ccc(C)nc1] | Methyl 2-(6- methylpyridin- 3-yl)-5- nitrobenzoate | ¹H NMR (500 MHz, DMSO-d6) δ [ppm] 8.60 (d, J = 2.5 Hz, 1H), 8.46 (dd, J = 8.5, 2.5 Hz, 1H), 8.43 (d, J = 2.3 Hz, 1H), 7.77 (d, J = 8.5 Hz, 1H), 7.71 (dd, J = 8.0, 2.4 Hz, 1H), 7.36 (d, J = 8.0 Hz, 1H), 3.72 (s, 3H), 2.54 (s, 3H). LCMS (Analytical Method A): Rₜ = 0.96 min; MS (ESIPos) m/z = 273.0 (M + H)⁺. |
| 10A | 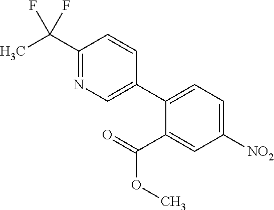 [SMILES: COC(=O)c1cc([N+](=O)[O-])ccc1-c1ccc(C(C)(F)F)nc1] | Methyl 2-[6- (1,1- difluoroethyl) pyridin-3-yl]-5- nitrobenzoate | ¹H NMR (500 MHz, DMSO-d6) δ [ppm] 8.69-8.66 (m, 2H), 8.51 (dd, J = 8.5, 2.5 Hz, 1H), 8.01 (dd, J = 8.1, 2.3 Hz, 1H), 7.83-7.79 (m, 2H), 3.74 (s, 3H), 2.06 (t, J = 19.1 Hz, 3H). LCMS (Analytical Method A): Rₜ = 1.19 min; MS (ESIPos) m/z = 323.0 (M + H)⁺. |
| 11A | 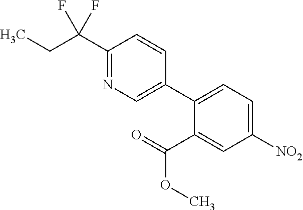 [SMILES: CCC(F)(F)c1ccc(-c2ccc([N+](=O)[O-])cc2C(=O)OC)cn1] | Methyl 2-[6- (1,1- difluoropropyl) pyridin-3-yl]-5- nitrobenzoate | ¹H NMR (250 MHz, DMSO-d6) δ [ppm] 8.68-8.69 (m, 2H), 8.52 (dd, J = 8.5, 2.5 Hz, 1H), 8.02 (dd, J = 8.1, 2.2 Hz, 1H), 7.84 (d, J = 8.5 Hz, 1H), 7.79 (d, J = 8.1 Hz, 1H), 3.73 (s, 3H), 2.47-2.30 (m, 2H), 0.97 (t, J = 7.5 Hz, 3H). LCMS (Analytical Method A): Rₜ = 1.24 min; MS (ESIPos) m/z = 337.0 (M + H)⁺. |

### Intermediate 12A: Methyl 2-(3-tert-butyl-1H-pyrazol-1-yl)-5-nitrobenzoate

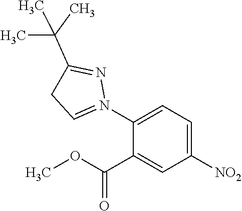

To a pressure tube were added 3-tert-butyl-1H-pyrazole (500 mg, 4.026 mmol), methyl 2-fluoro-5-nitrobenzoate (882 mg, 4.429 mmol), acetonitrile (20 mL) and K₂CO₃ (1.67 g, 12.08 mmol) at RT. The tube was sealed and the mixture heated at 90° C. for 29 h. The reaction mixture was diluted with EE, filtered and the filtrate concentrated under reduced pressure. The residue was purified by Biotage Isolera™ chromatography (using a gradient of eluents; 0-30% EE in heptane) to give the title compound (1.219 g, 86% yield) as a yellow oil.

¹H NMR (500 MHz, Chloroform-d) δ [ppm] 8.52 (d, J=2.6 Hz, 1H), 8.37 (dd, J=8.9, 2.6 Hz, 1H), 7.74 (d, J=2.6 Hz, 1H), 7.62 (d, J=8.9 Hz, 1H), 6.40 (d, J=2.6 Hz, 1H), 3.86 (s, 3H), 1.34 (s, 9H).

LCMS (Analytical Method D): Rₜ=4.61 min; MS (ESIPos) m/z=304.1 (M+H)⁺.

In analogy to the procedure described for Intermediate 8A, the following intermediates were prepared using methyl 2-bromo-5-nitrobenzoate and the appropriate boronic acids or, respectively, the corresponding pinacol boronic esters as starting materials.

| Int. | Structure | Name | Analytical Data |
|---|---|---|---|
| 13A | 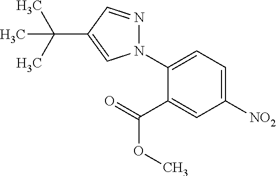 [SMILES: COC(=O)c1cc([N+](=O)[O-])ccc1-n1cc(C(C)(C)C)cn1] | Methyl 2-(4- tert-butyl-1H- pyrazol-1-yl)-5- nitrobenzoate | ¹H NMR (500 MHz, Chloroform-d) δ [ppm] 8.55 (d, J = 2.6 Hz, 1H), 8.38 (dd, J = 8.9, 2.6 Hz, 1H), 7.70-7.64 (m, 2H), 7.57 (d, J = 0.6 Hz, 1H), 3.84 (s, 3H), 1.32 (s, 9H). LCMS (Analytical Method D): Rₜ = 4.55 min; MS (ESIPos) m/z = 304.1 (M + H)⁺. |

### Intermediate 14A: Methyl 5-amino-2-(1-cyclobutyl-1H-pyrazol-4-yl)benzoate

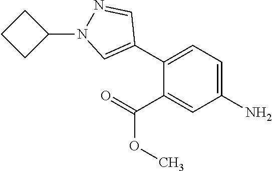 [SMILES: COC(=O)c1cc(N)ccc1-c1cnn(C2CCC2)c1]

Methyl 2-(1-cyclobutyl-1H-pyrazol-4-yl)-5-nitrobenzoate (Int. 8A, 1.32 g, 4.4 mmol) was dissolved in methanol (30 mL) and palladium on carbon (10% w/w; 45 mg) was added. The resulting mixture was stirred under a hydrogen atmosphere overnight. The following day the mixture was filtered over Celite® and washed with methanol (50 mL) and then concentrated at reduced pressure. The residue was purified by Biotage Isolera™ chromatography (using a gradient of eluents; 5-80% EE in heptane) giving the desired product (920 mg, 77% yield) as a yellow oil.

¹H NMR (500 MHz, DMSO-d6) δ [ppm] 7.73 (d, J=0.7 Hz, 1H), 7.37 (d, J=0.6 Hz, 1H), 7.11 (d, J=8.3 Hz, 1H), 6.80 (d, J=2.5 Hz, 1H), 6.70 (dd, J=8.3, 2.5 Hz, 1H), 5.31 (s, 2H), 4.86-4.73 (m, 1H), 3.69 (s, 3H), 2.48-2.31 (m, 4H), 1.83-1.71 (m, 2H).

LCMS (Analytical Method A): Rₜ=1.00 min; MS (ESIPos) m/z=272 (M+H)⁺.

In analogy to the procedure described for Intermediate 14A, the following intermediates were prepared using Pd/C hydrogenation from the corresponding nitrobenzene as starting material.

| Int. | Structure | Name | Analytical Data |
|---|---|---|---|
| 15A | 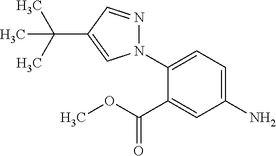 [SMILES: COC(=O)c1cc(N)ccc1-n1cc(C(C)(C)C)cn1] | Methyl 5- amino-2-(4-tert- butyl-1H- pyrazol-1- yl)benzoate | ¹H NMR (250 MHz, DMSO-d6) δ [ppm] 7.65 (d, J = 0.8 Hz, 1H), 7.46 (d, J = 0.7 Hz, 1H), 7.18 (d, J = 8.5 Hz, 1H), 6.84 (d, J = 2.6 Hz, 1H), 6.74 (dd, J = 8.5, 2.6 Hz, 1H), 5.54 (s, 2H), 3.53 (s, 3H), 1.25 (s, 9H). LCMS (Analytical Method D): Rₜ 3.87 min; MS (ESIPos) m/z = 274.0 (M + H)⁺. |
| 16A | 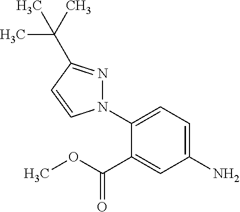 [SMILES: COC(=O)c1cc(N)ccc1-n1ccc(C(C)(C)C)n1] | Methyl 5- amino-2-(3-tert- butyl-1H- pyrazol-1- yl)benzoate | ¹H NMR (250 MHz, DMSO-d6) δ [ppm] 7.76 (d, J = 2.4 Hz, 1H), 7.17 (d, J = 8.5 Hz, 1H), 6.82 (d, J = 2.5 Hz, 1H), 6.73 (dd, J = 8.5, 2.6 Hz, 1H), 6.24 (d, J = 2.4 Hz, 1H), 5.51 (s, 2H), 3.54 (s, 3H), 1.23 (s, 9H). LCMS (Analytical Method D): Rₜ 3.85 min; MS (ESIPos) m/z = 274.1 (M + H)⁺. |
| 17A | 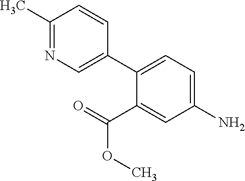 [SMILES: COC(=O)c1cc(N)ccc1-c1ccc(C)nc1] | Methyl 2-(6- methylpyridin- 3-yl)-5- nitrobenzoate | ¹H NMR (500 MHz, Chloroform-d) δ [ppm] 8.37 (d, J = 2.3 Hz, 1H), 7.45 (dd, J = 7.9, 2.4 Hz, 1H), 7.17 (d, J = 2.5 Hz, 1H), 7.12 (d, J = 7.9 Hz, 1H), 7.08 (d, J = 8.2 Hz, 1H), 6.81 (dd, J = 8.2, 2.6 Hz, 1H), 3.90 (s, 2H), 3.64 (s, 3H), 2.56 (s, 3H). LCMS (Analytical Method A): Rₜ = 0.72 min; MS (ESIPos) m/z = 243 (M + H)⁺. |
| 18A | 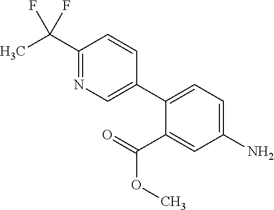 [SMILES: COC(=O)c1cc(N)ccc1-c1ccc(C(C)(F)F)nc1] | Methyl 5- amino-2-[6-(1,1- difluoroethyl) pyridin-3- yl]benzoate | ¹H NMR (500 MHz, Chloroform-d) δ [ppm] 8.58-8.47 (m, 1H), 7.68 (dd, J = 8.1, 2.2 Hz, 1H), 7.63 (d, J = 8.1 Hz, 1H), 7.25 (d, J = 2.5 Hz, 1H), 7.12 (d, J = 8.2 Hz, 1H), 6.87 (dd, J = 8.2, 2.6 Hz, 1H), 3.91 (s, 2H), 3.67 (s, 3H), 2.06 (t, J = 18.6 Hz, 3H). LCMS (Analytical Method A): Rₜ = 1.06 min; MS (ESIPos) m/z = 293 (M + H)⁺. |
| 19A | 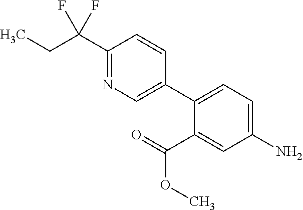 [SMILES: CCC(F)(F)c1ccc(-c2ccc(N)cc2C(=O)OC)cn1] | Methyl 5- amino-2-[6-(1,1- difluoropropyl) pyridin-3- yl]benzoate | ¹H NMR (250 MHz, DMSO-d6) δ [ppm] 8.48 (br. s, 1H), 7.76 (dd, J = 8.1, 2.2 Hz, 1H), 7.64 (d, J = 8.1 Hz, 1H), 7.15 (d, J = 8.3 Hz, 1H), 7.09 (d, J = 2.4 Hz, 1H), 6.83 (dd, J = 8.3, 2.4 Hz, 1H), 5.65 (s, 2H), 3.61 (s, 3H), 2.44-2.27 (m, 2H), 0.95 (t, J = 7.5 Hz, 3H). LCMS (Analytical Method A): Rₜ = 1.11 min; MS (ESIPos) m/z = 307.5 (M + H)⁺. |

### Intermediate 20A—2-Bromo-3-fluoro-5-nitrobenzoic acid

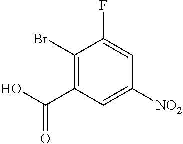 [SMILES: O=C(O)c1cc([N+](=O)[O-])cc(F)c1Br]

To a cooled solution of 2-bromo-3-fluoro-benzoic acid (5.00 g, 22.83 mmol) in sulfuric acid (45.5 mL) at 0° C. was added potassium nitrate portionwise (2.31 g, 22.83 mmol) over 5 minutes. The resulting solution turned yellow and was stirred at ambient temperature for 3 hours. The reaction mixture was poured onto ice and the resultant off-white precipitate was filtered, washed with water and dried in vacuo overnight to afford the title compound (1.50 g, 24% yield) as an off-white solid. ¹H NMR (250 MHz, DMSO-d6) δ [ppm] 8.43 (dd, J=8.2 & 2.1 Hz, 1H), 8.38-8.36 (m, 1H).

LCMS (Analytical Method A): Rₜ=0.87 min; MS (ESIneg) m/z=261.9/263.9 (M−H)⁻, Br isotope pattern.

### Intermediate 21A—Ethyl 2-bromo-3-fluoro-5-nitrobenzoate

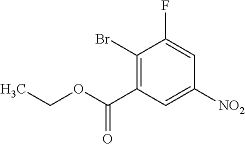 [SMILES: CCOC(=O)c1cc([N+](=O)[O-])cc(F)c1Br]

A solution of 2-bromo-3-fluoro-5-nitrobenzoic acid (Int. 20A, 1.5 g, 5.68 mmol) and sulfuric acid (0.3 mL) in EtOH (12.4 mL) was heated at 100° C. for 16 h. After this time the reaction mixture cooled to room temperature and then diluted with EE and 2M aqueous sodium hydroxide solution.

The organic phase isolated and the aqueous layer back-extracted with further EE. The organic layers were combined, washed with saturated aqueous sodium chloride solution, dried (MgSO₄), filtered and concentrated in vacuo to afford the title compound (1.45 g, 80% yield) as an orange solid.

¹H NMR (250 MHz, DMSO-d6) δ [ppm] 8.49 (dd, J=8.3, 2.6 Hz, 1H), 8.40 (dd, J=2.6, 1.4 Hz, 1H), 4.41 (q, J=7.1 Hz, 2H), 1.36 (t, J=7.1 Hz, 3H).

LCMS (Analytical Method A): Rₜ=1.22 min; no mass ion observed.

### Intermediate 22A—Ethyl 5-amino-2-bromo-3-fluorobenzoate

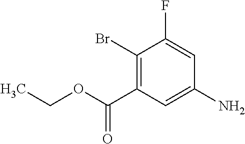

A mixture of ethyl 2-bromo-3-fluoro-5-nitrobenzoate (Int. 21A, 0.80 g, 2.74 mmol) and palladium on carbon (10% w/w; 146 mg) in EE/EtOH (27 mL; 8:2 v:v) were stirred under a hydrogen atmosphere for 16 hours. The reaction mixture was filtered through Celite® and concentrated at reduced pressure. The residue was purified by Biotage Isolera™ chromatography (using a gradient of eluents; 0-40% EE in heptane) giving the title compound (330 mg, 43% yield) as an orange oil. ¹H NMR (250 MHz, DMSO-d6) δ [ppm] 6.79 (dd, J=2.6, 1.0 Hz, 1H), 6.62 (dd, J=11.4, 2.6 Hz, 1H), 5.87 (s, 2H), 4.30 (q, J=7.1 Hz, 2H), 1.31 (t, J=7.1 Hz, 3H).

LCMS (Analytical Method A): Rₜ=1.11 min; MS (ESIPos) m/z=261.8/263.8 (M+H)⁺, Br isotope pattern.

### Intermediate 23A—Ethyl 5-amino-3-fluoro-2-[6-(trifluoromethyl)pyridin-3-yl]benzoate

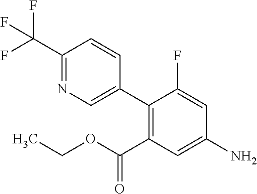 [SMILES: CCOC(=O)c1cc(N)cc(F)c1-c1ccc(C(F)(F)F)nc1]

To a pressure tube was added [6-(trifluoromethyl)pyridin-3-yl]boronic acid (361 mg, 1.89 mmol), ethyl 5-amino-2-bromo-3-fluorobenzoate (Int. 22A, 330 mg, 1.26 mmol), palladium(II) acetate (14 mg, 0.06 mmol), 2-dicyclohexylphosphino-2′,6′-dimethoxybiphenyl (52 mg, 0.13 mmol) and potassium phosphate (802 mg, 3.78 mmol) in tetrahydrofuran (6.3 mL). The tube was degassed with nitrogen, sealed and the reaction mixture heated at 60° C. for 16 h. The reaction mixture was cooled to room temperature and then partitioned between EE and saturated aqueous sodium hydrogen carbonate solution. The aqueous layer was removed and the organic layer was washed with saturated aqueous sodium chloride solution, dried (MgSO₄), filtered and concentrated at reduced pressure. The residue was purified by Biotage Isolera™ chromatography (using a gradient of eluents; 0-50% EE in heptane) giving the title compound (475 mg, 77% yield) as a pale yellow solid.

¹H NMR (250 MHz, DMSO-d6) δ [ppm] 8.57 (s, 1H), 7.92-7.91 (m, 2H), 7.02 (d, J=2.1 Hz, 1H), 6.66 (dd, J=12.4, 2.3 Hz, 1H), 6.02 (s, 2H), 4.01 (q, J=7.1 Hz, 2H), 0.92 (t, J=7.1 Hz, 3H). LCMS (Analytical Method A): Rₜ=1.19 min; MS (ESIPos) m/z=329.0 (M+H)⁺.

In analogy to Intermediate 23A, the following intermediates were prepared using the corresponding aryl bromide and appropriate boronic acids or, respectively, the corresponding pinacol boronic esters as starting materials.

| Int. | Structure | Name | Analytical Data |
|---|---|---|---|
| 24A | 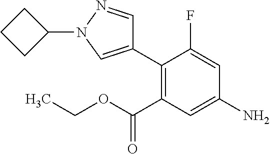 [SMILES: CCOC(=O)c1cc(N)cc(F)c1-c1cnn(C2CCC2)c1] | Ethyl 5-amino- 2-(1-cyclobutyl- 1H-pyrazol-4- yl)-3- fluorobenzoate | ¹H NMR (250 MHz, DMSO-d6) δ [ppm] 7.70 (s, 1H), 7.33 (s, 1H), 6.66 (d, J = 2.3 Hz, 1H), 6.53 (dd, J = 12.6, 2.3 Hz, 1H), 5.67 (s, 2H), 4.83 (m, 1H), 4.11 (q, J = 7.1 Hz, 2H), 2.45-2.28 (m, 4H), 1.87-1.69 (m, 2H), 1.07 (t, J = 7.1 Hz, 3H). LCMS (Analytical Method A): Rₜ = 1.11 min; MS (ESIPos) m/z = 304.1 (M + H)⁺. |

Alternatively, Intermediate 24A can be synthesized by the procedure described below via Intermediates 25A through 28A.

### Intermediate 25A—3-Fluoro-2-hydroxy-5-nitrobenzoic acid

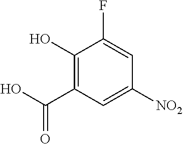 [SMILES: O=C(O)c1cc([N+](=O)[O-])cc(F)c1O]

3-Fluoro-2-hydroxybenzoic acid (24 g, 153 mmol) was dissolved in concentrated sulfuric acid (240 mL) and cooled to 0° C. Concentrated nitric acid (11.5 mL, 181 mmol, 69% solution) was then added dropwise over 30 minutes and the internal temperature was maintained below 10° C. After stirring at 0° C. for a further 60 minutes the mixture was poured onto ice water and the desired product precipitated as an off white solid. This was filtered, washed with water (500 mL) and dried in the vacuum oven giving the desired product (26.0 g, 84% yield) as a tan solid.

¹H NMR (250 MHz, DMSO-d6) δ [ppm] 8.39 (dd, J=2.8, 1.3 Hz, 1H), 8.23 (dd, J=10.8, 2.9 Hz, 1H).

LCMS (Analytical Method A): Rₜ=0.93 min; MS (ESINeg) m/z=200 (M−H)⁻.

### Intermediate 26A—Ethyl 3-fluoro-2-hydroxy-5-nitrobenzoate

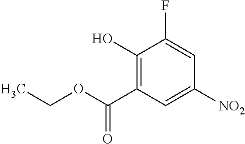 [SMILES: CCOC(=O)c1cc([N+](=O)[O-])cc(F)c1O]

3-Fluoro-2-hydroxy-5-nitrobenzoic acid (Int. 25A, 26.0 g, 129 mmol) was dissolved in ethanol (520 mL) and concentrated sulfuric acid (7.0 mL, 129 mmol) was added and the resulting solution was heated at reflux for 4 days. The mixture was then allowed to cool to room temperature and product precipitated as a white solid. This was filtered and washed with heptane (150 mL) giving the desired product (9.0 g, 30% yield) as white needles. The filtrate was concentrated at reduced pressure and the residue obtained was dissolved in TBME (300 mL) and washed with water (100 mL) and brine (2×100 mL), dried (Na₂SO₄), filtered and concentrated at reduced pressure. The residue was purified by Biotage Isolera™ chromatography (using a gradient of eluents; 5-30% TBME in heptane) giving further product (12.0 g, 41% yield) as an off-white solid. The fractions were combined to give the title compound (21.0 g, 71% yield) as an off-white solid.

¹H NMR (500 MHz, DMSO-d6) δ [ppm] 8.42-8.32 (m, 2H), 4.40 (q, J=7.1 Hz, 2H), 1.36 (t, J=7.1 Hz, 3H).

LCMS (Analytical Method A): Rₜ=1.25 min; MS (ESIneg) m/z=228 (M−H)⁻.

### Intermediate 27A—Ethyl 3-fluoro-5-nitro-2-{[(trifluoromethyl)sulfonyl]oxy}benzoate

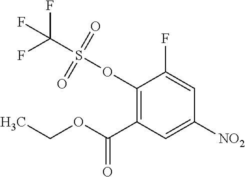 [SMILES: CCOC(=O)c1cc([N+](=O)[O-])cc(F)c1OS(=O)(=O)C(F)(F)F]

Ethyl 3-fluoro-2-hydroxy-5-nitrobenzoate (Int. 26A, 15.0 g, 65.4 mmol) was stirred in dichloromethane (300 mL) and cooled to 0° C. and triethylamine (11.0 mL, 78.5 mmol) was added giving a bright yellow solution. Trifluoromethanesulfonic anhydride (12.2 mL, 72.0 mmol) was then added dropwise and the internal temperature was maintained below 10° C. and upon complete addition a colourless solution was observed. Further triethylamine (2.7 mL, 19.6 mmol) followed by trifluoromethanesulfonic anhydride (2.2 mL, 13.0 mmol) were added at 0° C. and the resulting mixture was stirred for 10 minutes. The mixture was allowed to warm to room temperature then washed with 1M aqueous HCl (2×150 mL) and brine (100 mL), dried (Na₂SO₄), filtered and concentrated at reduced pressure. The residue was purified by Biotage Isolera™ chromatography (using a gradient of eluents; 2-20% TBME in heptane) giving the title compound (21.6 g, 91% yield) as a pale yellow oil.

¹H NMR (500 MHz, DMSO-d6) δ [ppm] 8.88 (dd, J=9.6, 2.8 Hz, 1H), 8.56 (dd, J=2.7, 1.9 Hz, 1H), 4.44 (q, J=7.1 Hz, 2H), 1.36 (t, J=7.1 Hz, 3H).

LCMS (Analytical Method A): Rₜ=1.33 min; no mass ion observed.

### Intermediate 28A—Ethyl 2-(1-cyclobutyl-1H-pyrazol-4-yl)-3-fluoro-5-nitrobenzoate

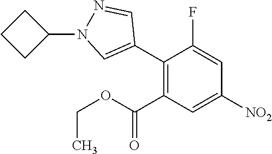 [SMILES: CCOC(=O)c1cc([N+](=O)[O-])cc(F)c1-c1cnn(C2CCC2)c1]

A biphasic mixture of 1-cyclobutyl-4-(4,4,5,5-tetramethyl-1,3,2-dioxaborolan-2-yl)-1H-pyrazole (7.56 g, 30.45 mmol), Pd(PPh₃)₂Cl₂ (388 mg, 0.55 mmol), K₂CO₃ (7.65 g, 55.37 mmol) and ethyl 3-fluoro-5-nitro-2-{[(trifluoromethyl)sulfonyl]oxy}benzoate (10.00 g, 27.68 mmol) was split equally between 8 pressure tubes and dissolved in DME/water (10:1, 8×17.3 mL) and the resulting solutions were degassed with nitrogen for 5 minutes. The reaction vessels were sealed and heated to 100° C. for 1 hour. The reaction mixtures were then cooled to room temperature, combined and diluted with ethyl acetate and washed with 1M aqueous sodium hydroxide solution, then saturated aqueous sodium chloride solution, dried (MgSO₄), filtered and concentrated at reduced pressure. The residue was purified by Biotage Isolera™ chromatography (using a gradient of eluents; 0-15% EE in heptane) giving the title compound (8.65 g, 94% yield) as a pale yellow oil.

¹H NMR (250 MHz, Chloroform-d) δ [ppm] 8.34 (dd, J=2.3, 1.2 Hz, 1H), 8.10 (dd, J=9.6, 2.4 Hz, 1H), 7.77 (d, J=2.2 Hz, 1H), 7.68 (s, 1H), 4.84 (m, 1H), 4.35 (q, J=7.1 Hz, 2H), 2.71-2.47 (m, 4H), 2.04-1.84 (m, 2H), 1.30 (t, J=7.1 Hz, 3H).

LCMS (Analytical Method A): Rₜ=1.30 min; MS (ESIPos) m/z=334.0 (M+H)⁺.

### Intermediate 24A: Ethyl 5-amino-2-(1-cyclobutyl-1H-pyrazol-4-yl)-3-fluorobenzoate

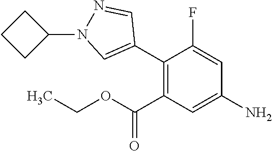 [SMILES: CCOC(=O)c1cc(N)cc(F)c1-c1cnn(C2CCC2)c1]

A mixture of ethyl 2-(1-cyclobutyl-1H-pyrazol-4-yl)-3-fluoro-5-nitrobenzoate (Int. 28A, 8.65 g, 25.95 mmol) and palladium on carbon (10% w/w; 1.38 g) in EE/EtOH (230 mL; 8:2 v:v) was stirred under a hydrogen atmosphere for 16 hours. The reaction mixture was then filtered through Celite® (washing with ethyl acetate) and concentrated at reduced pressure. The residual pale yellow oil was allowed to crystallise and the solid material was triturated with diethyl ether and isolated by filtration to afford the title compound (5.85 g, 73% yield) as an off-white solid. The filtrate obtained was concentrated at reduced pressure and the residue was purified by Biotage Isolera™ chromatography (using a gradient of eluents; 0-40% EE in heptane) and the pale yellow crystalline solid obtained was triturated with diethyl ether to remove the coloration to afford the title compound (1.35 g, 17% yield) as an off-white solid. The fractions were combined to give the title compound (7.2 g, 90% yield) as an off-white solid.

¹H NMR (250 MHz, DMSO-d6) δ [ppm] 7.70 (s, 1H), 7.33 (s, 1H), 6.66 (s, 1H), 6.59-6.48 (m, 1H), 5.67 (s, 2H), 4.84 (m, 1H), 4.11 (q, J=7.1 Hz, 2H), 2.47-2.29 (m, 4H), 1.87-1.69 (m, 2H), 1.08 (t, J=7.1 Hz, 3H).

LCMS (Analytical Method A): Rₜ=1.17 min; MS (ESIPos) m/z=304.0 (M+H)⁺.

### Intermediate 29A: 2-Bromo-4-fluoro-5-nitrobenzoic acid

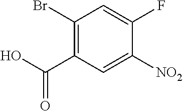 [SMILES: O=C(O)c1cc([N+](=O)[O-])c(F)cc1Br]

To a cooled solution of 2-bromo-4-fluorobenzoic acid (5.00 g, 22.83 mmol) in sulfuric acid (42.5 mL) at 0° C. was added potassium nitrate portionwise (2.31 g, 22.83 mmol) over 5 minutes with the resulting solution stirred at ambient temperature for 3 h. After this time the reaction mixture was poured onto ice and the resultant precipitate was filtered, washing with water and dried in vacuo for 60 h to afford a mixture of regioisomers favouring the title compound (4:1; 5.03 g, 70% yield) as an off-white solid.

¹H NMR (250 MHz, DMSO-d6) δ [ppm] 8.51 (d, J=8.0 Hz, 1H), 8.17 (d, J=10.9 Hz, 1H).

LCMS (Analytical Method A): Rₜ=0.91 min; MS (ESIPos) m/z=261.8/263.8 (M−H)⁻, Br isotope pattern.

### Intermediate 30A: 5-Amino-2-bromo-4-fluorobenzoic acid

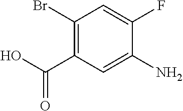 [SMILES: Nc1cc(C(=O)O)c(Br)cc1F]

To a solution of 2-bromo-4-fluoro-5-nitrobenzoic acid (Int. 29A, 1.56 g, 5.91 mmol) in EE/EtOH (59 mL; 8:2 v:v) under N₂ (evacuated under vacuum and purged with nitrogen thrice) was added palladium on carbon (10% w/w; 314 mg). The reaction flask was evacuated and charged with hydrogen (repeat two further times), after which the reaction flask was isolated under an atmosphere of hydrogen and allowed to stir for 16 h. After this time the reaction flask was evacuated and charged with nitrogen (thrice), with the reaction mixture filtered through Celite® (washing with ethyl acetate) and the reaction mixture concentrated in vacuo. The residue was purified by Biotage Isolera™ chromatography (using a gradient of eluents; 0-30% EE in heptane) to afford the desired product (1.40 g, 62% yield) as an orange oil.

¹H NMR (250 MHz, DMSO-d6) δ [ppm] 7.37 (d, J=11.0 Hz, 1H), 7.25 (d, J=9.4 Hz, 1H).

LCMS (Analytical Method A): Rₜ=0.89 min; MS (ESIPos) m/z=233.7/235.7 (M+H)⁺, Br isotope pattern.

### Intermediate 31A: Ethyl 5-amino-2-bromo-4-fluorobenzoate

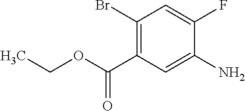

A solution of 5-amino-2-bromo-4-fluorobenzoic acid (Int. 30A, 1.4 g, 3.65 mmol) and sulfuric acid (0.20 mL) in EtOH (8 mL) was heated at 100° C. for 16 h. After this time the reaction mixture was diluted with EE and 2M aqueous sodium hydroxide solution, with the organic phase isolated and the aqueous layer back-extracted with further EE. The organic layers were combined, washed with saturated aqueous sodium chloride solution, dried (MgSO₄), filtered and concentrated in vacuo. The residue was purified by Biotage Isolera™ chromatography (using a gradient of eluents; 0-20% EE in heptane) to afford the title compound (443 mg, 40% yield) as a pale pink crystalline solid.

¹H NMR (500 MHz, Chloroform-d) δ [ppm] 7.32-7.28 (m, 2H), 4.39 (q, J=7.1 Hz, 2H), 3.85 (s, 2H), 1.41 (t, J=7.1 Hz, 3H).

LCMS (Analytical Method A) Rₜ=1.24 min; MS (ESIPos) m/z=261.70/263.70 (M+H)⁺, Br isotope pattern.

### Intermediate 32A: Ethyl 5-amino-4-fluoro-2-[6-(trifluoromethyl)pyridin-3-yl]benzoate

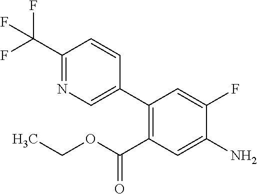 [SMILES: CCOC(=O)c1cc(N)c(F)cc1-c1ccc(C(F)(F)F)nc1]

A mixture of [6-(trifluoromethyl)pyridin-3-yl]boronic acid (484 mg, 2.54 mmol), ethyl 5-amino-2-bromo-3-fluorobenzoate (Int. 31A, 509 mg, 1.69 mmol, 87% purity), palladium(II) acetate (19 mg, 0.09 mmol), 2-dicyclohexylphosphino-2′,6′-dimethoxybiphenyl (69 mg, 0.17 mmol) and potassium phosphate (1.08 g, 5.07 mmol) in tetrahydrofuran (8.4 mL) in a pressure tube was degassed with nitrogen (via balloon) for 5 minutes. After this time, the balloon was removed, the tube sealed and the reaction mixture warmed to 60° C. for 16 h. After this time, the reaction mixture was allowed to cool to RT and was partitioned between EE and saturated aqueous sodium hydrogen carbonate solution. The aqueous layer was removed and the organic layer was washed with saturated aqueous sodium chloride solution, dried (MgSO₄), filtered and concentrated in vacuo. The residue was purified by Biotage Isolera™ chromatography (using a gradient of eluents; 0-50% EE in heptane) to afford the title compound (510 mg, 51% yield) as a pale tan solid.

¹H NMR (250 MHz, Chloroform-d) δ [ppm] 8.62 (d, J=1.6 Hz, 1H), 7.78 (dd, J=8.1, 1.6 Hz, 1H), 7.71 (d, J=8.1 Hz, 1H), 7.49 (d, J=8.9 Hz, 1H), 6.98 (d, J=11.1 Hz, 1H), 4.13 (q, J=7.1 Hz, 2H), 4.02 (s, 2H), 1.07 (t, J=7.1 Hz, 3H).

LCMS (Analytical Method A): Rₜ=1.17 min; MS (ESIPos) m/z=329.0 (M+H)⁺.

### Intermediate 33A: Ethyl trans 2-(3-chlorophenyl)cyclopropanecarboxylate

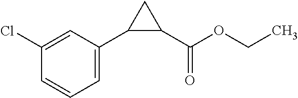

A mixture of [Rh(OAc)₂]₂ (57 mg, 0.13 mmol) and 3-chlorostyrene (10.0 mL, 78.6 mmol) in DCM (100 mL) was cooled to 0° C. Ethyl diazoacetate (3.2 mL, 26.2 mmol) was then added dropwise over 10 minutes. The reaction was stirred for overnight, until nitrogen evolution ceased, then the reaction mixture was concentrated in vacuo. The trans:cis (2:1) mixture was purified twice by Biotage Isolera™ chromatography (using a gradient of eluents; 0-10% EE in heptane) to give the title compound as a single diastereomer (1.48 g, 25% yield) as a colourless oil.

¹H NMR (500 MHz, Chloroform-d) δ [ppm] 7.24-7.13 (m, 2H), 7.09-7.05 (m, 1H), 7.01-6.96 (m, 1H), 4.17 (q, J=7.1 Hz, 2H), 2.55-2.43 (m, 1H), 1.93-1.85 (m, 1H), 1.65-1.57 (m, 1H), 1.33-1.23 (m, 4H).

LCMS (Analytical Method A): Rₜ=1.32 min; no ionisation was observed.

### Intermediate 34A: trans-2-(3-Chlorophenyl)cyclopropanecarboxylic acid

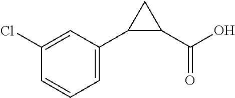 [SMILES: O=C(O)C1CC1c1cccc(Cl)c1]

To a solution of ethyl trans 2-(3-chlorophenyl)cyclopropanecarboxylate (Int. 33A, 1.48 g, 6.6 mmol) in tetrahydrofuran (20 mL) was added 2M aqueous sodium hydroxide (15 mL) and the reaction stirred at 65° C. for 5 h. After this time, the reaction was cooled to room temperature, acidified with 2M aqueous HCl (15 mL), extracted with EE (50 mL). The organics were then washed with brine (25 mL), dried (Na₂SO₄), filtered and concentrated in vacuo to give the title compound (1.23 g, 94% yield) as a pale yellow solid.

¹H NMR (500 MHz, DMSO-d6) δ [ppm] 12.36 (s, 1H), 7.33-7.19 (m, 3H), 7.15-7.08 (m, 1H), 2.45-2.37 (m, 1H), 1.89-1.79 (m, 1H), 1.46-1.30 (m, 2H).

LCMS (Analytical Method A): Rₜ=1.10 min; no ionisation was observed.

In analogy to Intermediate 33A and 34A, the following ester and carboxylic acid intermediates were prepared:

| Int. | Structure | Name | Analytical Data |
|---|---|---|---|
| 35A | 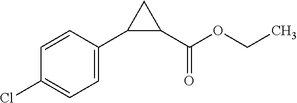 | Ethyl trans-2- (4- chlorophenyl) cyclopropane- carboxylate | ¹H NMR (500 MHz, Chloroform-d) δ [ppm] 7.24-7.13 (m, 2H), 7.09-7.05 (m, 1H), 7.01-6.96 (m, 1H), 4.17 (q, J = 7.1 Hz, 2H), 2.55-2.43 (m, 1H), 1.93-1.85 (m, 1H), 1.65-1.57 (m, 1H), 1.33-1.23 (m, 4H). LCMS (Analytical Method A) Rₜ = 1.32 min; MS (ESIPos) m/z = 224.9/226.9 (M + H)⁺, Cl isotope pattern. |
| 36A | 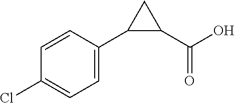 [SMILES: O=C(O)C1CC1c1ccc(Cl)cc1] | trans-2-(4- chlorophenyl) cyclopropane- carboxylic acid | ¹H NMR (500 MHz, Chloroform-d) δ [ppm] 7.27-7.24 (m, 2H), 7.06-7.02 (m, 2H), 2.57 (ddd, J = 9.4, 6.6, 4.1 Hz, 1H), 1.87 (ddd, J = 8.5, 5.2, 4.2 Hz, 1H), 1.70-1.62 (m, 1H), 1.37 (ddd, J = 8.4, 6.7, 4.7 Hz, 1H). LCMS (Analytical Method F): Rₜ = 2.78 min; no ionisation was observed. |

700 mg of Intermediate 34A was separated into enantiomers by preparative chiral HPLC (>95% e.e.). The absolute stereochemistry of both enantiomers is unknown.

Preparative conditions: Instrument: Berger Prep SFC; Stationary Phase: Chiralpak IC 5 μm, 250×20 mm; Mobile phase: CO₂/isopropanol+0.5% isopropylamine 88/12; Flowrate: 70 mL/min; UV detection: @=210 nm; Temperature: 40° C.; Pressure: 100 bars

Analytical conditions: Instrument: SFC Berger; Column: Chiralpak IC 5 μm, 4.6×300 mm; Mobile phase: CO₂/isopropanol+0.5% isopropylamine 92/8; Flowrate: 2.4 mL/min; Temperature: 40° C.; UV detection: @=210 nm; Pressure: 100 bars; Injection: 10 L of a 1 mg/mL solution in acetonitrile

These separated trans enantiomers were used in further steps as:

### Intermediate 37A (First Trans Enantiomer)

220 mg—NMR showed 60 mol % of isopropylamine present

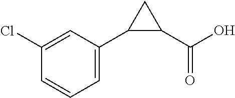 [SMILES: O=C(O)C1CC1c1cccc(Cl)c1]

Rₜ=27.4 min 100.0% purity

### Intermediate 38A (Second Trans Enantiomer)

134 mg—NMR showed 60 mol % of isopropylamine present.

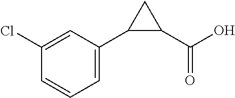 [SMILES: O=C(O)C1CC1c1cccc(Cl)c1]

Rₜ=30.2 min 100.0% purity 800 mg of Intermediate 36A were separated into enantiomers by preparative chiral HPLC (>95% ee). The absolute stereochemistry of both enantiomers is unknown.

Preparative conditions: Instrument: Waters SFC 200 Prep; Stationary Phase: Chiralpak AD-H 5 μm, 250×20 mm; Mobile phase: CO₂/methanol+0.5% isopropylamine 75/25; Flowrate: 50 g/min; UV detection: A=210 nm; Temperature: 40° C.; Pressure: 100 bars

Analytical conditions: Instrument: SFC Berger; Column: Chiralpak AD-H 5 μm, 4.6×250 mm; Mobile phase: CO₂/methanol+0.5% isopropylamine 75/25; Flowrate: 2.4 mL/min; Temperature: 40° C.; UV detection: A=210 nm; Pressure: 100 bars

These separated enantiomers were used in further steps as:

### Intermediate 39A (First Trans Enantiomer)

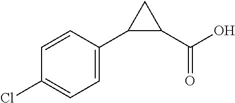 [SMILES: O=C(O)C1CC1c1ccc(Cl)cc1]

Rₜ=2.5 min 100.0% purity

To remove residual isopropylamine, the material was dissolved in ethyl acetate (50 mL) and washed with 1M aqueous HCl (2×20 mL) and brine (20 mL), then dried (Na₂SO₄), filtered and concentrated at reduced pressure giving the product (309 mg) as a white solid.

### Intermediate 40A (Second Trans Enantiomer)

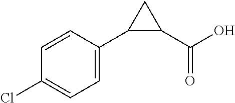 [SMILES: O=C(O)C1CC1c1ccc(Cl)cc1]

Rₜ=2.8 min 98.5% purity

To remove residual isopropylamine, the material was dissolved in ethyl acetate (50 mL) and washed with 1M aqueous HCl (2×20 mL) and brine (20 mL), then dried (Na₂SO₄), filtered and concentrated at reduced pressure giving the product (307 mg) as a white solid.

### Intermediate 41A: Methyl 5-({[2-(3-chlorophenyl)cyclopropyl]carbonyl}amino)-2-(1-cyclobutyl-1H-pyrazol-4-yl)benzoate, as a Mixture of Trans Enantiomers

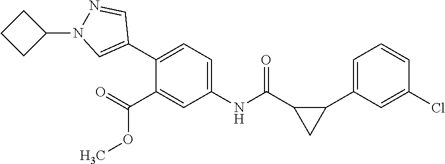 [SMILES: COC(=O)c1cc(NC(=O)C2CC2c2cccc(Cl)c2)ccc1-c1cnn(C2CCC2)c1]

Methyl 5-amino-2-(1-cyclobutyl-1H-pyrazol-4-yl)benzoate (Intermediate 14A, 69 mg, 0.25 mmol) and trans-2-(3-chlorophenyl)cyclopropanecarboxylic acid (Int. 34A, 60 mg, 0.31 mmol) were dissolved in DMF (2 mL) and N,N-diisopropylethylamine (0.09 mL, 0.51 mmol) and HATU (116 mg, 0.31 mmol) was added giving a light brown solution. This was stirred at 80° C. for 3 h. The mixture was partitioned between EE and water. The aqueous layer was extracted with EE (3×15 mL). The combined organic layers were washed with 2M aqueous HCl (10 mL) and brine (10 mL), dried (Na₂SO₄), filtered and concentrated. The residue was purified by Biotage Isolera™ chromatography to give the title compound (94 mg, 82% yield) as a white solid.

¹H NMR (400 MHz, DMSO-d6) δ [ppm] 1.42-1.54 (m, 2H), 1.72-1.82 (m, 2H), 2.07-2.11 (m, 1H), 2.33-2.48 (m, 5H), 3.75 (s, 3H), 4.84 (m, 1H), 7.18 (m, 1H), 7.25-7.34 (m, 3H), 7.43 (d, 1H), 7.50 (s, 1H), 7.67 (dd, 1H), 7.92 (s, 1H), 7.95 (d, 1H), 10.46 (s, 1H).

LCMS (method 1): Rₜ=1.33 min; MS (ESIPos) m/z=450 (M+H)⁺.

In analogy to Intermediate 41A, the following examples were prepared using the corresponding amine and carboxylic acid as starting materials:

| Int. | Structure | Name | Analytical Data |
|---|---|---|---|
| 42A | 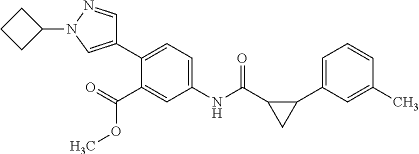 [SMILES: COC(=O)c1cc(NC(=O)C2CC2c2cccc(C)c2)ccc1-c1cnn(C2CCC2)c1] | Methyl 2-(1- cyclobutyl-1H- pyrazol-4-yl)-5- ({[2-(3- methylphenyl) cyclopropyl] carbonyl}amino) benzoate, as a mixture of trans enantiomers | ¹H NMR (400 MHz, DMSO-d6) δ [ppm] 1.35-1.39 (m, 1H), 1.46-1.51 (m, 1H), 1.73-1.82 (m, 2H), 2.02-2.07 (m, 1H), 2.28 (s, 3H), 2.33-2.48 (m, 5H), 3.75 (s, 3H), 4.83 (m, 1H), 6.97-7.02 (m, 3H), 7.18 (m, 1H), 7.43 (d, 1H), 7.50 (s, 1H), 7.67 (dd, 1H), 7.91 (s, 1H), 7.95 (d, 1H), 10.44 (s, 1H). LCMS (method 1): Rₜ = 1.31 min; MS (ESIPos) m/z = 430 (M + H)⁺. |
| 43A | 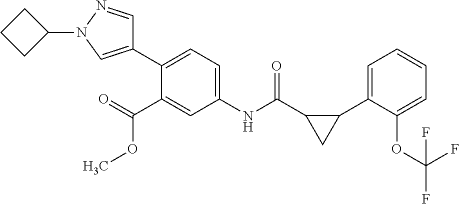 [SMILES: COC(=O)c1cc(NC(=O)C2CC2c2ccccc2OC(F)(F)F)ccc1-c1cnn(C2CCC2)c1] | Methyl 2-(1- cyclobutyl-1H- pyrazol-4-yl)-5- [(-2-[2- (trifluoromethoxy) phenyl] cyclopropyl} carbonyl) amino]benzoate, as a mixture of trans enantiomers | ¹H NMR (250 MHz, Chloroform-d) δ [ppm] 7.88 (s, 1H), 7.72-7.50 (m, 4H), 7.34 (d, J = 8.4 Hz, 1H), 7.26-7.18 (m, 3H), 7.10-6.97 (m, 1H), 4.85-4.66 (m, 1H), 3.78 (s, 3H), 2.85-2.69 (m, 1H), 2.69-2.36 (m, 4H), 2.00-1.65 (m, 4H), 1.46-1.31 (m, 1H). LCMS (Analytical Method A): Rₜ = 1.34 min; MS (ESIPos) m/z = 500 (M + H)⁺. |
| 44A | 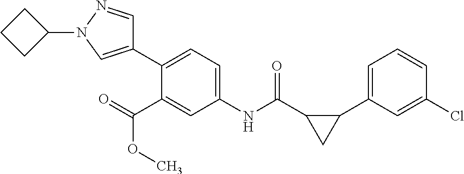 [SMILES: COC(=O)c1cc(NC(=O)C2CC2c2cccc(Cl)c2)ccc1-c1cnn(C2CCC2)c1] | Methyl 5-({[2- (3- chlorophenyl) cyclopropyl] carbonyl} amino)-2- (1-cyclobutyl- 1H-pyrazol-4- yl)benzoate, as a single trans enantiomer | ¹H NMR (500 MHz, Chloroform-d) δ [ppm] 7.88 (s, 1H), 7.72-7.61 (m, 1H), 7.61-7.54 (m, 2H), 7.54-7.43 (m, 1H), 7.38-7.32 (m, 1H), 7.25-7.15 (m, 2H), 7.11-7.06 (m, 1H), 7.06- 6.99 (m, 1H), 4.85-4.69 (m, 1H), 3.79 (s, 3H), 2.67-2.44 (m, 5H), 1.96-1.79 (m, 2H), 1.79-1.70 (m, 2H), 1.45- 1.32 (m, 1H). LCMS (Analytical Method A): Rₜ = 1.33 min; MS (ESIPos) m/z = 450 (M + H)⁺. |
| 45A | 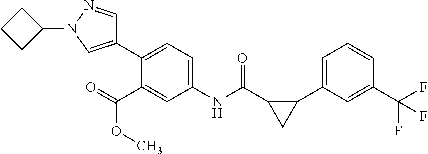 [SMILES: COC(=O)c1cc(NC(=O)C2CC2c2cccc(C(F)(F)F)c2)ccc1-c1cnn(C2CCC2)c1] | Methyl 2-(1- cyclobutyl-1H- pyrazol-4-yl)-5- [({(2-[3- (trifluoromethyl) phenyl] cyclopropyl} carbonyl) amino]benzoate, as a mixture of trans enantiomers | ¹H NMR (500 MHz, Chloroform-d) δ [ppm] 7.88 (s, 1H), 7.72-7.63 (m, 1H), 7.61-7.53 (m, 2H), 7.52-7.45 (m, 2H), 7.45-7.38 (m, 1H), 7.38-7.30 (m, 3H), 4.83-4.71 (m, 1H), 3.79 (s, 3H), 2.72-2.64 (m, 1H), 2.63-2.53 (m, 2H), 2.53-2.45 (m, 2H), 1.93- 1.83 (m, 2H), 1.82-1.76 (m, 2H), 1.45- 1.37 (m, 1H). LCMS (Analytical Method A): Rₜ = 1.37 min; MS (ESIPos) m/z = 484 (M + H)⁺. |
| 46A | 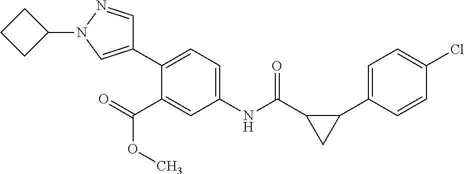 [SMILES: COC(=O)c1cc(NC(=O)C2CC2c2ccc(Cl)cc2)ccc1-c1cnn(C2CCC2)c1] | Methyl 5-({[2- (4- chlorophenyl) cyclopropyl] carbonyl} amino)-2- (1-cyclobutyl- 1H-pyrazol-4- yl)benzoate, as a mixture of trans enantiomers | ¹H NMR (400 MHz, DMSO-d6) δ [ppm] 1.37-1.42 (m, 1H), 1.49-1.53 (m, 1H), 1.73-1.82 (m, 2H), 2.03-2.07 (m, 1H), 2.33-2.50 (m, 5H), 3.75 (s, 3H), 4.84 (quint, 1H), 7.21-7.25 (m, 2H), 7.34- 7.37 (m, 2H), 7.43 (d, 1H), 7.50 (s, 1H), 7.68 (dd, 1H), 7.92 (s, 1H), 7.95 (d, 1H), 10.47 (s, 1H). LCMS (method 1): Rₜ = 1.32 min; MS (ESIPos) m/z = 450 (M + H)⁺. |
| 47A | 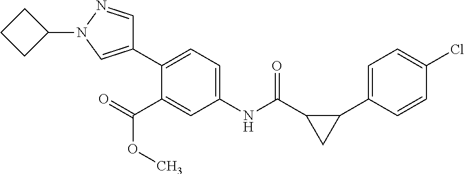 [SMILES: COC(=O)c1cc(NC(=O)C2CC2c2ccc(Cl)cc2)ccc1-c1cnn(C2CCC2)c1] | Methyl 5-({[2- (4- chlorophenyl) cyclopropyl] carbonyl} amino)-2- (1-cyclobutyl- 1H-pyrazol-4- yl)benzoate, as a single trans enantiomer | ¹H NMR (250 MHz, Chloroform-d) δ [ppm] 8.11 (s, 1H), 7.96-7.82 (m, 1H), 7.71-7.48 (m, 3H), 7.35-7.12 (m, 3H), 6.99 (d, J = 8.5 Hz, 2H), 4.85- 4.64 (m, 1H), 3.75 (s, 3H), 2.68-2.34 (m, 5H), 1.97-1.61 (m, 4H), 1.35- 1.17 (m, 1H). LCMS (Analytical Method A): Rₜ = 1.42 min; MS (ESIPos) m/z = 450 (M + H)⁺. |
| 48A | 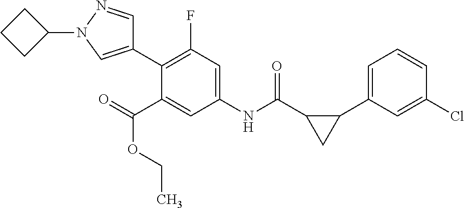 [SMILES: CCOC(=O)c1cc(NC(=O)C2CC2c2cccc(Cl)c2)cc(F)c1-c1cnn(C2CCC2)c1] | Ethyl 5-({[2-(3- chlorophenyl) cyclopropyl] carbonyl} amino)-2- (1-cyclobutyl- 1H-pyrazol-4- yl)-3- fluorobenzoate, as single trans enantiomer 1 | ¹H NMR (500 MHz, Chloroform-d) δ [ppm] 7.81-7.73 (m, 1H), 7.61-7.57 (m, 1H), 7.56-7.52 (m, 2H), 7.44- 7.41 (m, 1H), 7.21-7.19 (m, 2H), 7.09 (s, 1H), 7.04-7.03 (m, 1H), 4.82-4.69 (m, 1H), 4.25-4.18 (m, 2H), 2.64- 2.47 (m, 5H), 1.95-1.80 (m, 2H), 1.80- 1.72 (m, 2H), 1.42-1.36 (m, 1H), 1.22-1.19 (m, 3H). LCMS (Analytical Method A): Rₜ = 1.39 min; MS (ESIPos) m/z = 482 (M + H)⁺. |
| 49A | 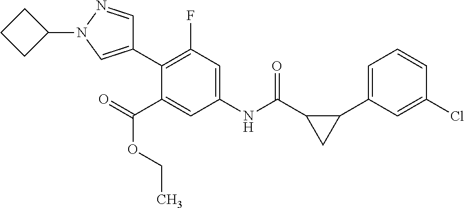 [SMILES: CCOC(=O)c1cc(NC(=O)C2CC2c2cccc(Cl)c2)cc(F)c1-c1cnn(C2CCC2)c1] | Ethyl 5-({[2-(3- chlorophenyl) cyclopropyl] carbonyl} amino)-2- (1-cyclobutyl- 1H-pyrazol-4- yl)-3- fluorobenzoate, as single trans enantiomer 2 | ¹H NMR (500 MHz, Chloroform-d) δ [ppm] 7.81-7.71 (m, 1H), 7.65-7.51 (m, 3H), 7.45-7.41 (m, 1H), 7.22- 7.19 (m, 2H), 7.09-7.06 (m, 1H), 7.05- 7.03 (m, 1H), 4.83-4.68 (m, 1H), 4.22 (q, J = 7.1 Hz, 2H), 2.65-2.47 (m, 5H), 1.98-1.79 (m, 2H), 1.79-1.68 (m, 2H), 1.46-1.36 (m, 1H), 1.21- 1.18 (m, 3H). LCMS (Analytical Method A): Rₜ = 1.38 min; MS (ESIPos) m/z = 482 (M + H)⁺. |
| 50A | 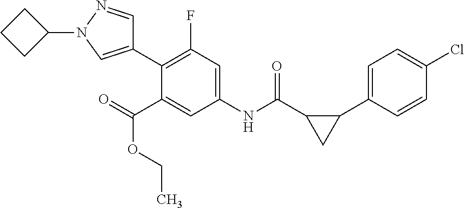 [SMILES: CCOC(=O)c1cc(NC(=O)C2CC2c2ccc(Cl)cc2)cc(F)c1-c1cnn(C2CCC2)c1] | Ethyl 5-({[2-(4- chlorophenyl) cyclopropyl] carbonyl} amino)-2- (1-cyclobutyl- 1H-pyrazol-4- yl)-3- fluorobenzoate, as a mixture of trans enantiomers | ¹H NMR (250 MHz, Chloroform-d) δ [ppm] 7.69 (d, J = 10.8 Hz, 1H), 7.61 (s, 1H), 7.51 (d, J = 1.3 Hz, 1H), 7.46 (s, 1H), 7.36 (s, 1H), 7.19 (d, J = 8.5 Hz, 2H), 6.97 (d, J = 8.5 Hz, 2H), 4.78-4.61 (m, 1H), 4.14 (q, J = 7.1 Hz, 2H), 2.59- 2.34 (m, 4H), 1.90-1.74 (m, 2H), 1.72- 1.58 (m, 2H), 1.55-1.52 (m, 1H), 1.34- 1.22 (m, 1H), 1.11 (t, J = 7.1 Hz, 3H). LCMS (Analytical Method A) Rₜ = 1.41 min; MS (ESIPos) m/z = 482.05 (M + H)⁺. |
| 50B | 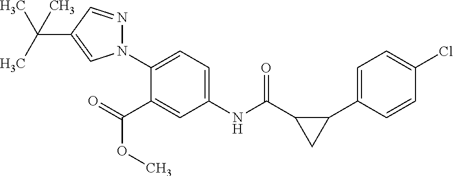 [SMILES: COC(=O)c1cc(NC(=O)C2CC2c2ccc(Cl)cc2)ccc1-n1cc(C(C)(C)C)cn1] | Methyl 2-(4-tert- butyl-1H- pyrazol-1-yl)-5- ({[2-(4- chlorophenyl) cyclopropyl] carbonyl} amino) benzoate, as a mixture of trans enantiomers | ¹H NMR (500 MHz, Chloroform-d) δ [ppm] 8.73 (s, 1H), 7.77-7.66 (m, 2H), 7.47 (s, 1H), 7.39 (s, 1H), 7.28 (d, J = 8.5 Hz, 1H), 7.25-7.21 (m, 2H), 7.05-6.97 (m, 2H), 3.61 (s, 3H), 2.52 (ddd, J = 9.6, 6.4, 4.1 Hz, 1H), 1.86-1.78 (m, 1H), 1.73-1.64 (m, 1H), 1.28 (s, 9H), 1.27- 1.25 (m, 1H); LCMS (Analytical Method A): Rₜ = 1.37 min; MS (ESIPos) m/z = 452.05 (M + H)⁺. |
| 51A | 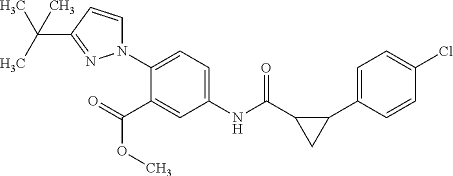 [SMILES: COC(=O)c1cc(NC(=O)C2CC2c2ccc(Cl)cc2)ccc1-n1ccc(C(C)(C)C)n1] | Methyl 2-(3-tert- butyl-1H- pyrazol-1-yl)-5- ({[2-(4- chlorophenyl) cyclopropyl] carbonyl} amino) benzoate, as a mixture of trans enantiomers | ¹H NMR (500 MHz, Chloroform-d) δ [ppm] 8.49 (s, 1H), 7.84-7.75 (m, 2H), 7.56 (d, J = 2.4 Hz, 1H), 7.30 (d, J = 9.7 Hz, 1H), 7.27-7.23 (m, 2H), 7.05-7.01 (m, 2H), 6.30 (d, J = 2.4 Hz, 1H), 3.66 (s, 3H), 2.56 (ddd, J = 9.5, 6.5, 4.1 Hz, 1H), 1.82-1.75 (m, 1H), 1.74-1.67 (m, 1H), 1.33 (s, 9H), 1.32-1.28 (m, 1H). LCMS (Analytical Method A): Rₜ = 1.39 min; MS (ESIPos) m/z = 452.05 (M + H)⁺. |
| 52A | 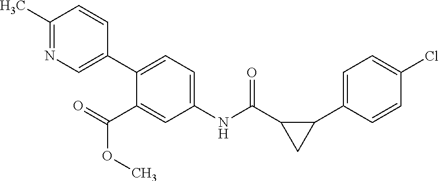 [SMILES: COC(=O)c1cc(NC(=O)C2CC2c2ccc(Cl)cc2)ccc1-c1ccc(C)nc1] | Methyl 5-({[2- (4- chlorophenyl) cyclopropyl] carbonyl} amino)-2- (6- methylpyridin- 3-yl)benzoate, as a mixture of trans enantiomers | ¹H NMR (500 MHz, Methanol-d4) δ [ppm] 8.27 (d, J = 2.0 Hz, 1H), 8.16 (d, J = 2.3 Hz, 1H), 7.82 (dd, J = 8.4, 2.3 Hz, 1H), 7.62 (dd, J = 8.0, 2.3 Hz, 1H), 7.34- 7.29 (m, 2H), 7.28-7.24 (m, 2H), 7.17- 7.12 (m, 2H), 3.67 (s, 3H), 2.55 (s, 3H), 2.48 (ddd, J = 9.5, 6.4, 4.0 Hz, 1H), 2.05 (ddd, J = 8.4, 5.2, 4.2 Hz, 1H), 1.67- 1.59 (m, 1H), 1.37 (ddd, J = 8.3, 6.4, 4.5 Hz, 1H). LCMS (Analytical Method A): Rₜ = 1.17 min; MS (ESIPos) m/z = 421.0 (M + H)⁺. |
| 53A | 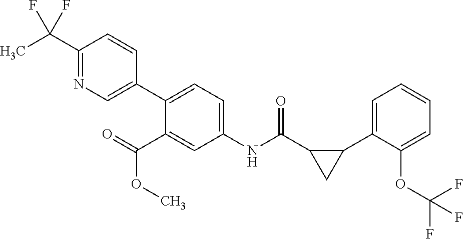 [SMILES: COC(=O)c1cc(NC(=O)C2CC2c2ccccc2OC(F)(F)F)ccc1-c1ccc(C(C)(F)F)nc1] | Methyl 2-[6- (1,1- difluoroethyl) pyridin-3-yl]-5- [(2-[2- (trifluoromethoxy) phenyl] cyclopropyl} carbonyl) amino]benzoate, as a mixture of trans enantiomers | ¹H NMR (250 MHz, Chloroform-d) δ [ppm] 8.58-8.49 (m, 1H), 8.14-8.03 (m, 1H), 7.96-7.81 (m, 1H), 7.81-7.61 (m, 2H), 7.63-7.50 (m, 1H), 7.34-7.21 (m, 3H), 7.13-7.03 (m, 1H), 3.70 (s, 3H), 2.87-2.72 (m, 1H), 2.07 (t, J = 18.6 Hz, 3H), 1.88-1.68 (m, 2H), 1.46-1.36 (m, 1H). LCMS (Analytical Method A): Rₜ = 1.34 min; MS (ESIPos) m/z = 521 (M + H)⁺. |
| 54A | 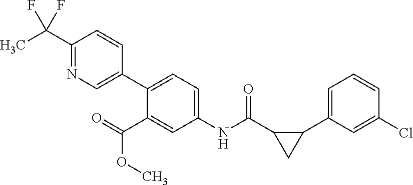 [SMILES: COC(=O)c1cc(NC(=O)C2CC2c2cccc(Cl)c2)ccc1-c1ccc(C(C)(F)F)nc1] | Methyl 5-({[(2- (3- chlorophenyl) cyclopropyl] carbonyl} amino)-2- [6-(1,1- difluoroethyl) pyridin-3- yl]benzoate, as a mixture of trans enantiomers | ¹H NMR (250 MHz, Chloroform-d) δ [ppm] 8.58-8.47 (m, 1H), 8.13-8.03 (m, 1H), 7.92-7.79 (m, 1H), 7.76-7.55 (m, 3H), 7.33-7.27 (m, 1H), 7.25-7.17 (m, 2H), 7.13-6.99 (m, 2H), 3.69 (s, 3H), 2.70-2.54 (m, 1H), 2.06 (t, J = 18.6 Hz, 3H), 1.82-1.68 (m, 2H), 1.50-1.36 (m, 1H). LCMS (Analytical Method A): Rₜ = 1.34 min; MS (ESIPos) m/z = 471 (M + H)⁺. |
| 55A | 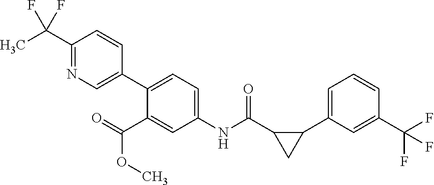 [SMILES: COC(=O)c1cc(NC(=O)C2CC2c2cccc(C(F)(F)F)c2)ccc1-c1ccc(C(C)(F)F)nc1] | Methyl 2-[6- (1,1- difluoroethyl) pyridin-3-yl]-5- [({2-[3- trifluoromethyl) phenyl] cyclopropyl} carbonyl) amino]benzoate, as a mixture of trans enantiomers | ¹H NMR (250 MHz, Chloroform-d) δ [ppm] 8.54 (s, 1H), 8.13-8.07 (m, 1H), 7.86 (d, J = 8.5 Hz, 1H), 7.78-7.57 (m, 3H), 7.54-7.38 (m, 2H), 7.38-7.27 (m, 3H), 3.70 (s, 3H), 2.76-2.63 (m, 1H), 2.06 (t, J = 18.6 Hz, 3H), 1.92-1.75 (m, 2H), 1.51-1.37 (m, 1H). LCMS (Analytical Method A): Rₜ = 1.36 min; MS (ESIPos) m/z = 505 (M + H)⁺. |
| 56A | 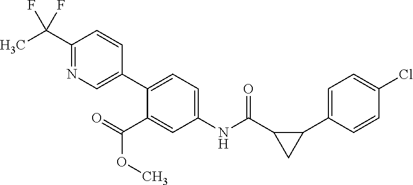 [SMILES: COC(=O)c1cc(NC(=O)C2CC2c2ccc(Cl)cc2)ccc1-c1ccc(C(C)(F)F)nc1] | Methyl 5-({[2- (4- chlorophenyl) cyclopropyl] carbonyl} amino)-2- [6-(1,1- difluoroethyl) pyridin-3- yl]benzoate, as a mixture of trans enantiomers | ¹H NMR (250 MHz, Chloroform-d) δ [ppm] 8.53 (d, J = 1.2 Hz, 1H), 8.12- 8.04 (m, 1H), 7.86 (d, J = 8.4 Hz, 1H), 7.76-7.62 (m, 2H), 7.59 (s, 1H), 7.33- 7.22 (m, 3H), 7.11-7.02 (m, 2H), 3.70 (s, 3H), 2.69-2.55 (m, 1H), 2.06 (t, J = 18.6 Hz, 3H), 1.82-1.69 (m, 2H), 1.46- 1.34 (m, 1H). LCMS (Analytical Method A): Rₜ = 1.33 min; MS (ESIPos) m/z = 471 (M + H)⁺. |
| 57A | 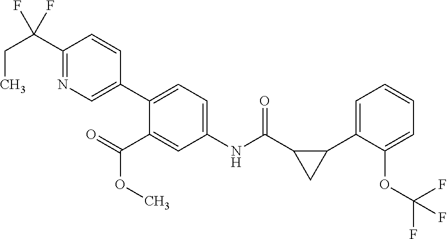 [SMILES: CCC(F)(F)c1ccc(-c2ccc(NC(=O)C3CC3c3ccccc3OC(F)(F)F)cc2C(=O)OC)cn1] | Methyl 2-[6- (1,1- difluoropropyl) pyridin-3-yl]-5- [({2-[2- (trifluoromethoxy) phenyl] cyclopropyl} carbonyl) amino]benzoate, as a mixture of trans enantiomers | 1H NMR (500 MHz, DMSO-d6) δ [ppm] 10.64 (s, 1H), 8.57 (d, J = 1.6 Hz, 1H), 8.26 (d, J = 2.1 Hz, 1H), 7.93-7.82 (m, 2H), 7.76-7.67 (m, 1H), 7.46 (d, J = 8.4 Hz, 1H), 7.42-7.34 (m, 3H), 7.28-7.22 (m, 1H), 3.65 (s, 3H), 2.58-2.55 (m, 1H), 2.44-2.29 (m, 2H), 2.12-2.07 (m, 1H), 1.59-1.52 (m, 2H), 0.96 (t, J = 7.5 Hz, 3H). LCMS (Analytical Method A): Rₜ = 1.38 min; MS (ESIPos) m/z = 535.5 (M + H)⁺. |
| 58A | 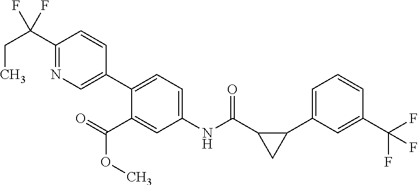 [SMILES: CCC(F)(F)c1ccc(-c2ccc(NC(=O)C3CC3c3cccc(C(F)(F)F)c3)cc2C(=O)OC)cn1] | Methyl 2-[6- (1,1- difluoropropyl) pyridin-3-yl]-5- [({2-[3- (trifluoromethyl) phenyl] cyclopropyl} carbonyl) amino]benzoate, as a mixture of trans enantiomers | ¹H NMR (500 MHz, DMSO-d6) δ [ppm] 10.65 (s, 1H), 8.59-8.54 (m, 1H), 8.26 (d, J = 2.1 Hz, 1H), 7.91-7.84 (m, 2H), 7.71 (d, J = 8.1 Hz, 1H), 7.61-7.54 (m, 4H), 7.46 (d, J = 8.4 Hz, 1H), 3.65 (s, 3H), 2.60-2.55 (m, 1H), 2.44-2.29 (m, 2H), 2.23-2.12 (m, 1H), 1.63-1.57 (m, 1H), 1.57-1.50 (m, 1H), 0.95 (t, J = 7.5 Hz, 3H). LCMS (Analytical Method A): Rₜ = 1.39 min; MS (ESIPos) m/z = 519.5 (M + H)⁺. |
| 59A | 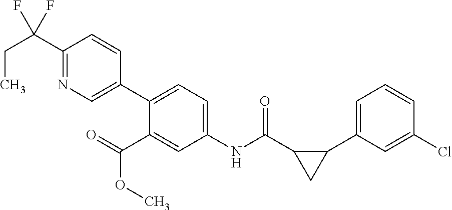 [SMILES: CCC(F)(F)c1ccc(-c2ccc(NC(=O)C3CC3c3cccc(Cl)c3)cc2C(=O)OC)cn1] | Methyl 5-({[2- (3- chlorophenyl) cyclopropyl] carbonyl} amino)-2- [6-(1,1- difluoropropyl) pyridin-3- yl]benzoate, as a mixture of trans enantiomers | ¹H NMR (500 MHz, DMSO-d6) δ [ppm] 10.63 (s, 1H), 8.59-8.54 (m, 1H), 8.26 (d, J = 2.1 Hz, 1H), 7.87 (m, 2H), 7.71 (d, J = 8.1 Hz, 1H), 7.46 (d, J = 8.4 Hz, 1H), 7.36-7.26 (m, 3H), 7.22-7.19 (m, 1H), 3.65 (s, 3H), 2.49-2.42 (m, 1H), 2.42-2.29 (m, 2H), 2.17-2.11 (m, 1H), 1.60-1.53 (m, 1H), 1.51-1.45 (m, 1H), 0.95 (t, J = 7.4 Hz, 3H). LCMS (Analytical Method A): Rₜ = 1.39 mins; MS (ESIPos) m/z = 485.1 (M + H)⁺. |
| 60A | 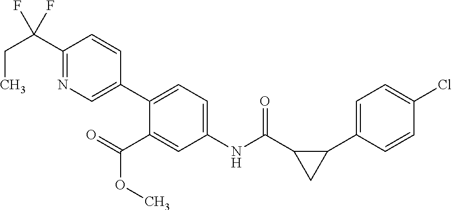 [SMILES: CCC(F)(F)c1ccc(-c2ccc(NC(=O)C3CC3c3ccc(Cl)cc3)cc2C(=O)OC)cn1] | Methyl 5-({[2- (4- chlorophenyl) cyclopropyl] carbonyl} amino)-2- [6-(1,1- difluoropropyl) pyridin-3- yl]benzoate, as a mixture of trans enantiomers | ¹H NMR (500 MHz, DMSO-d6) δ [ppm] 10.63 (s, 1H), 8.56 (d, J = 1.8 Hz, 1H), 8.25 (d, J = 2.2 Hz, 1H), 7.90-7.83 (m, 2H), 7.70 (d, J = 8.1 Hz, 1H), 7.45 (d, J = 8.4 Hz, 1H), 7.39-7.33 (m, 2H), 7.28- 7.21 (m, 2H), 3.64 (s, 3H), 2.44 (ddd, J = 9.5, 6.4, 4.1 Hz, 1H), 2.41-2.28 (m, 2H), 2.14-2.05 (m, 1H), 1.59-1.50 (m, 1H), 1.42 (ddd, J = 8.2, 6.4, 4.3 Hz, 1H), 0.95 (t, J = 7.5 Hz, 3H) LCMS (Analytical Method F): Rₜ = 4.31 min; MS (ESIPos) m/z = 485.0 (M + H)⁺. |
| 61A | 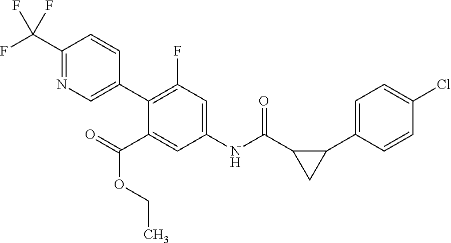 [SMILES: CCOC(=O)c1cc(NC(=O)C2CC2c2ccc(Cl)cc2)cc(F)c1-c1ccc(C(F)(F)F)nc1] | Ethyl 5-({[2-(4- chlorophenyl) cyclopropyl] carbonyl} amino)-3- fluoro-2-[6- (trifluoromethyl) pyridin-3- yl]benzoate, as a mixture of trans enantiomers | ¹H NMR (250 MHz, DMSO-d6) δ [ppm] 10.86 (s, 1H), 8.68 (s, 1H), 8.05 (d, J = 9.8 Hz, 1H), 8.02-7.92 (m, 3H), 7.38 (d, J = 8.5 Hz, 2H), 7.26 (d, J = 8.6 Hz, 2H), 4.05 (q, J = 7.1 Hz, 2H), 2.44 (m, 1H), 2.17-2.01 (m, 1H), 1.62-1.52 (m, 1H), 1.52-1.40 (m, 1H), 0.94 (t, J = 7.1 Hz, 3H). LCMS (Analytical Method A): Rₜ = 1.45 min; MS (ESIPos) m/z = 507.00 (M + H)⁺. |
| 62A | 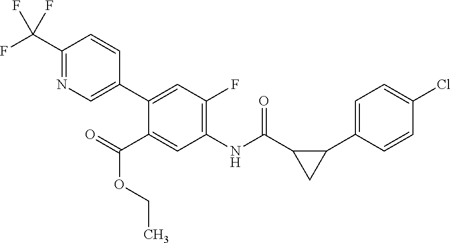 [SMILES: CCOC(=O)c1cc(NC(=O)C2CC2c2ccc(Cl)cc2)c(F)cc1-c1ccc(C(F)(F)F)nc1] | Ethyl 5-({[2-(4- chlorophenyl) cyclopropyl] carbonyl} amino)-4- fluoro-2-[6- (trifluoromethyl) pyridin-3- yl]benzoate, as a mixture of trans enantiomers | ¹H NMR (250 MHz, DMSO-d6) δ [ppm] 10.40 (s, 1H), 8.75 (d, J = 8.0 Hz, 1H), 8.71 (m, 1H), 8.08-8.01 (m, 1H), 7.96 (d, J = 7.7 Hz, 1H), 7.53 (d, J = 11.4 Hz, 1H), 7.38 (d, J = 8.9, 2H), 7.27-7.21 (m, 2H), 4.08 (q, J = 7.2 Hz, 2H), 2.48-2.44 (m, 2H), 1.62-1.50 (m, 1H), 1.47-1.35 (m, 1H), 1.03-0.94 (t, J = 7.2 Hz, 3H). LCMS (Analytical Method A): Rₜ = 1.43 min; MS (ESIPos) m/z = 507.05 (M + H)⁺. |

### Example 1: 5-({[2-(3-chlorophenyl)cyclopropyl]carbonyl}amino)-2-(1-cyclobutyl-1H-pyrazol-4-yl)benzoic acid, as a Mixture of Trans Enantiomers

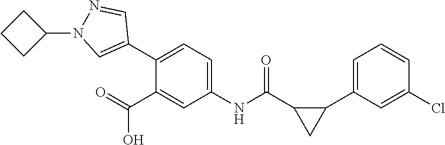 [SMILES: O=C(O)c1cc(NC(=O)C2CC2c2cccc(Cl)c2)ccc1-c1cnn(C2CCC2)c1]

To a stirred solution of methyl 5-({[2-(3-chlorophenyl)cyclopropyl]carbonyl}amino)-2-(1-cyclobutyl-1H-pyrazol-4-yl)benzoate, as a mixture of trans enantiomers (Intermediate 41A, 165 mg, 0.37 mmol) in THF (3 mL) was added LiOH (26 mg, 1.1 mmol, dissolved in 0.5 mL water). The reaction was stirred overnight at 80° C. The reaction mixture was then acidified at RT by addition of 1M aqueous HCl (2 mL) and extracted three times with ethyl acetate. The combined organic layers were washed with water and brine and dried with sodium sulfate. The solution was concentrated in vacuo and purified by preparative HPLC to afford the title compound (38 mg, 24% yield) as a white solid.

¹H NMR (400 MHz, DMSO-d6) δ [ppm] 1.42-1.46 (m, 1H), 1.49-1.54 (m, 1H), 1.73-1.82 (m, 2H), 2.07-2.12 (m, 1H), 2.33-2.47 (m, 5H), 4.82 (quint, 1H), 7.17-7.20 (m, 1H), 7.25-7.34 (m, 3H), 7.39 (d, 1H), 7.55 (s, 1H), 7.65 (dd, 1H), 7.86 (d, 1H), 7.93 (d, 1H), 10.41 (s, 1H).

LCMS (method 1): Rₜ=1.18 min; m/z (ESIPos)=436 (M+H)⁺.

### Example 2: 5-({[2-(4-chlorophenyl)cyclopropyl]carbonyl}amino)-2-(1-cyclobutyl-1H-pyrazol-4-yl)-3-fluorobenzoic acid, as a mixture of trans

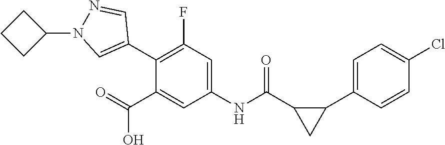 [SMILES: O=C(O)c1cc(NC(=O)C2CC2c2ccc(Cl)cc2)cc(F)c1-c1cnn(C2CCC2)c1]

A stirred solution of ethyl 5({[2-(4-chlorophenyl)cyclopropyl]carbonyl}amino)-2-(1-cyclobutyl-1H-pyrazol-4-yl)-3-fluorobenzoate, as a mixture of trans enantiomers (119 mg, 0.25 mmol) and lithium hydroxide monohydrate (20.7 mg, 0.49 mmol) in THF:water (2:1 v/v; 2.5 mL), was heated at 60° C. for 16 h. After this time, the reaction mixture was acidified by addition of 1M aqueous hydrogen chloride solution and partitioned between EE and saturated aqueous sodium chloride solution. The organic layer was isolated, dried (MgSO₄), filtered and concentrated in vacuo, with the residual material purified by preparative HPLC (Method A). The desired fractions were combined and the acetonitrile component removed in vacuo, with the resulting aqueous media acidified by dropwise addition of concentrated aqueous hydrogen chloride solution. The resulting precipitate was isolated by suction filtration to afford the title compound (mixture of trans enantiomers; 52 mg, 45% yield), as a white solid.

¹H NMR (250 MHz, DMSO-d6) δ [ppm] 10.61 (s, 1H), 7.90 (s, 1H), 7.73 (d, J=12.2 Hz, 1H), 7.58 (s, 1H), 7.50 (s, 1H), 7.36 (d, J=8.4 Hz, 2H), 7.24 (d, J=8.6 Hz, 2H), 4.98-4.76 (m, 1H), 2.45-2.27 (m, 5H), 2.13-1.95 (m, 1H), 1.90-1.68 (m, 2H), 1.60-1.48 (m, 1H), 1.48-1.33 (m, 1H).

LCMS (Analytical Method F): Rₜ=3.64 min; MS (ESIPos) m/z=454.1 (M+H)⁺.

40 mg of Example 2 were separated into enantiomers by preparative chiral HPLC.

Preparative conditions: Instrument: Sepiatec: Prep SFC100; column: Chiralpak ID 5 μm 250×30 mm; eluent A: CO₂, eluent B: 2-Propanol+0.4 Vol-% diethylamine (99%); isocratic: 33% B; flow 100.0 mL/min, temperature: 40° C.; BPR: 150 bar; MWD @ 254 nm

Analytical conditions: Instrument: Agilent: 1260, Aurora SFC-Modul; column: Chiralpak ID 5 μm 100×4.6 mm; eluent A: CO₂, eluent B: 2-Propanol+0.2 Vol-% diethylamine (99%); isocratic: 33% B; flow 4.0 mL/min; temperature: 37.5° C.; BPR: 100 bar; MWD @ 254 nm

### Example 3: (+)-5-({[trans-2-(4-chlorophenyl)cyclopropyl]carbonyl}amino)-2-(1-cyclobutyl-1H-pyrazol-4-yl)-3-fluorobenzoic acid

Enantiomer 1: Rₜ=2.99 min;
specific optical rotation: □=258° (589 nm, 20° C., c=1.0000 g/100 mL)

### Example 4: (−)-5-({[trans-2-(4-chlorophenyl)cyclopropyl]carbonyl}amino)-2-(1-cyclobutyl-1H-pyrazol-4-yl)-3-fluorobenzoic acid

Enantiomer 2: Rₜ=7.74 min;
specific optical rotation: □=−224° (589 nm, 20° C., c=1.0000 g/100 mL) In analogy to Example 1, the following examples were prepared using the corresponding ester as starting materials:

| Ex. | Structure | Name | Analytical Data |
|---|---|---|---|
| 5 | 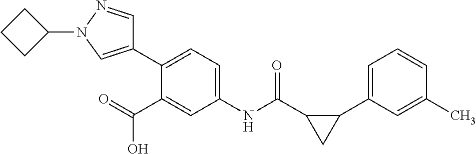 [SMILES: Cc1cccc(C2CC2C(=O)Nc2ccc(-c3cnn(C4CCC4)c3)c(C(=O)O)c2)c1] | 2-(1-cyclobutyl- 1H-pyrazol-4- yl)-5-({[2-(3- methylphenyl) cyclopropyl] carbonyl}amino) benzoic acid, as a mixture of trans enantiomers | ¹H NMR (400 MHz, DMSO-d6) δ [ppm] 1.34-1.39 (m, 1H), 1.46-1.50 (m, 1H), 1.72-1.81 (m, 2H), 2.03-2.07 (m, 1H), 2.28 (s, 3H), 2.32-2.47 (m, 5H), 4.82 (m, 1H), 6.97-7.02 (m, 3H), 7.17 (m, 1H), 7.39 (d, 1H), 7.56 (s, 1H), 7.65 (dd, 1H), 7.85 (d, 1H), 7.94 (s, 1H), 10.39 (s, 1H). LCMS (method 1): Rₜ = 1.16 min; MS (ESIPos) m/z = 416 (M + H)⁺. |
| 6 | 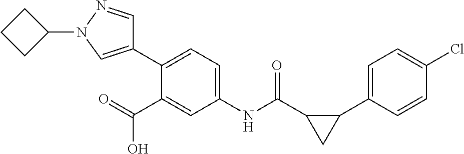 [SMILES: O=C(O)c1cc(NC(=O)C2CC2c2ccc(Cl)cc2)ccc1-c1cnn(C2CCC2)c1] | 5-({[2-(4- chlorophenyl) cyclopropyl] carbonyl}amino)-2- (1-cyclobutyl- 1H-pyrazol-4- yl)benzoic acid, as a mixture of trans enantiomers | ¹H NMR (400 MHz, DMSO-d6) δ [ppm] 1.36-1.41 (m, 1H), 1.49-1.53 (m, 1H), 1.73-1.82 (m, 2H), 2.03-2.08 (m, 1H), 2.34-2.47 (m, 5H), 4.82 (m, 1H), 7.23 (d, 2H), 7.35 (d, 2H), 7.39 (d, 1H), 7.55 (s, 1H), 7.66 (dd, 1H), 7.88 (d, 1H), 7.93 (s, 1H), 10.42 (s, 1H). LCMS (method 1): Rₜ = 1.18 min; MS (ESIPos) m/z = 436 (M + H)⁺. |
| 7 | 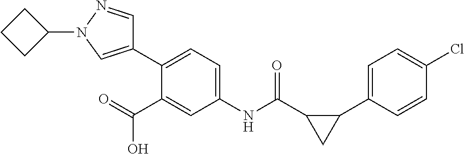 [SMILES: O=C(O)c1cc(NC(=O)C2CC2c2ccc(Cl)cc2)ccc1-c1cnn(C2CCC2)c1] | 5-({[2-(4- chlorophenyl) cyclopropyl] carbonyl}amino)- 2-(1-cyclobutyl- 1H-pyrazol-4- yl)benzoic acid, as a single trans enantiomer | ¹H NMR (500 MHz, DMSO-d6) δ [ppm] 12.94 (s, 1H), 10.40 (s, 1H), 7.92 (s, 1H), 7.87 (d, J = 2.2 Hz, 1H), 7.66 (dd, J = 8.5, 2.3 Hz, 1H), 7.55 (s, 1H), 7.39 (d, J = 8.5 Hz, 1H), 7.35 (d, J = 8.5 Hz, 2H), 7.23 (d, J = 8.5 Hz, 2H), 4.91- 4.77 (m, 1H), 2.48-2.34 (m, 5H), 2.10-2.02 (m, 1H), 1.86-1.71 (m, 2H), 1.55-1.45 (m, 1H), 1.42-1.34 (m, 1H). LCMS (Analytical Method F): Rₜ = 3.43 min; MS (ESIPos) m/z = 436 (M + H)⁺. |
| 8 | 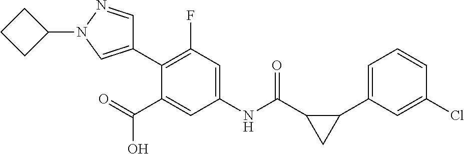 [SMILES: O=C(O)c1cc(NC(=O)C2CC2c2cccc(Cl)c2)cc(F)c1-c1cnn(C2CCC2)c1] | (+)-5-({[2-(3- chlorophenyl) cyclopropyl] carbonyl}amino)- 2-(1-cyclobutyl- 1H-pyrazol-4- yl)-3- fluorobenzoic acid, as single trans enantiomer 1 | ¹H NMR (500 MHz, DMSO-d6) δ [ppm] 10.55 (s, 1H), 7.93-7.88 (m, 1H), 7.72-7.63 (m, 1H), 7.57-7.51 (m, 1H), 7.51-7.44 (m, 1H), 7.35- 7.23 (m, 3H), 7.21-7.16 (m, 1H), 4.90-4.79 (m, 1H), 2.48-2.32 (m, 5H), 2.12-2.06 (m, 1H), 1.82-1.72 (m, 2H), 1.56-1.49 (m, 1H), 1.49-1.42 (m, 1H). LCMS (Analytical Method D): Rₜ = 2.09 min; MS (ESIPos) m/z = 454 (M + H)⁺. |
| 9 | 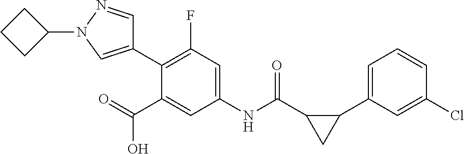 [SMILES: O=C(O)c1cc(NC(=O)C2CC2c2cccc(Cl)c2)cc(F)c1-c1cnn(C2CCC2)c1] | (−)-5-({[2-(3- chlorophenyl) cyclopropyl] carbonyl} amino)-2- (1-cyclobutyl- 1H-pyrazol-4- yl)-3- fluorobenzoic acid, as single trans enantiomer 2 | ¹H NMR (500 MHz, DMSO-d6) δ [ppm] 10.50 (s, 1H), 7.92 (s, 1H), 7.65- 7.56 (m, 2H), 7.42-7.35 (m, 1H), 7.35-7.29 (m, 1H), 7.29-7.24 (m, 2H), 7.20-7.15 (m, 1H), 4.87-4.75 (m, 1H), 2.47-2.30 (m, 5H), 2.14- 2.05 (m, 1H), 1.84-1.71 (m, 2H), 1.56- 1.49 (m, 1H), 1.49-1.41 (m, 1H). LCMS (Analytical Method D): Rₜ = 2.09 min; MS (ESIPos) m/z = 454 (M + H)⁺. |
| 10 | 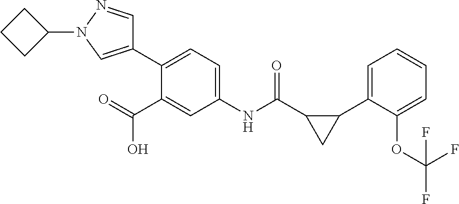 [SMILES: O=C(O)c1cc(NC(=O)C2CC2c2ccccc2OC(F)(F)F)ccc1-c1cnn(C2CCC2)c1] | 2-(1-cyclobutyl- 1H-pyrazol-4- yl)-5-[({2-[2- (trifluoromethoxy) phenyl] cyclopropyl} carbonyl) amino]benzoic acid, as a mixture of trans enantiomers | ¹H NMR (500 MHz, DMSO-d6) δ [ppm] 12.98 (s, 1H), 10.41 (s, 1H), 7.94 (s, 1H), 7.88 (d, J = 2.2 Hz, 1H), 7.66 (dd, J = 8.5, 2.3 Hz, 1H), 7.56 (s, 1H), 7.42-7.36 (m, 4H), 7.27-7.20 (m, 1H), 4.89-4.79 (m, 1H), 2.56-2.52 (m, 1H), 2.49-2.43 (m, 2H), 2.42-2.35 (m, 2H), 2.12-2.02 (m, 1H), 1.85-1.75 (m, 2H), 1.56-1.47 (m, 2H). LCMS (Analytical Method D): Rₜ = 5.01 min; MS (ESIPos) m/z = 486.05 (M + H)⁺. |
| 11 | 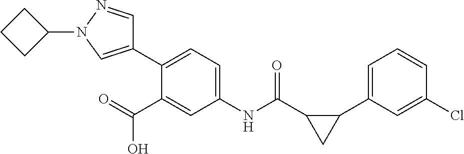 [SMILES: O=C(O)c1cc(NC(=O)C2CC2c2cccc(Cl)c2)ccc1-c1cnn(C2CCC2)c1] | 5-({[2-(3- chlorophenyl) cyclopropyl] carbonyl} amino)-2- (1-cyclobutyl- 1H-pyrazol-4- yl)benzoic acid, as a single trans enantiomer | ¹H NMR (500 MHz, DMSO-d6) δ [ppm] 10.32 (s, 1H), 7.96 (s, 1H), 7.74 (s, 1H), 7.60 (d, J = 17.1 Hz, 2H), 7.38- 7.29 (m, 2H), 7.29-7.23 (m, 2H), 7.21-7.15 (m, 1H), 4.85-4.74 (m, 1H), 2.46-2.29 (m, 5H), 2.18-2.06 (m, 1H), 1.83-1.72 (m, 2H), 1.55- 1.46 (m, 1H), 1.46-1.37 (m, 1H). LCMS (Analytical Method F): Rₜ = 3.45 min; MS (ESIPos) m/z = 436 (M + H)⁺. |
| 12 | 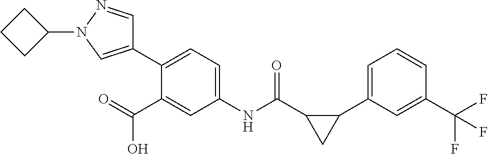 [SMILES: O=C(O)c1cc(NC(=O)C2CC2c2cccc(C(F)(F)F)c2)ccc1-c1cnn(C2CCC2)c1] | 2-(1-cyclobutyl- 1H-pyrazol-4- yl)-5-[({2-[3- (trifluoromethyl) phenyl] cyclopropyl} carbonyl) amino]benzoic acid, as a mixture of trans enantiomers | ¹H NMR (500 MHz, DMSO-d6) δ [ppm] 12.94 (s, 1H), 10.42 (s, 1H), 7.93 (d, J = 0.6 Hz, 1H), 7.90 (d, J = 2.3 Hz, 1H), 7.67 (dd, J = 8.5, 2.3 Hz, 1H), 7.61- 7.50 (m, 5H), 7.41 (d, J = 8.5 Hz, 1H), 4.89-4.76 (m, 1H), 2.57-2.52 (m, 1H), 2.49-2.45 (m, 2H), 2.43-2.34 (m, 2H), 2.18-2.10 (m, 1H), 1.85- 1.72 (m, 2H), 1.60-1.54 (m, 1H), 1.54- 1.48 (m, 1H). LCMS (Analytical Method D): Rₜ = 5.04 min; MS (ESIPos) m/z = 470.05 (M + H)⁺. |
| 13 | 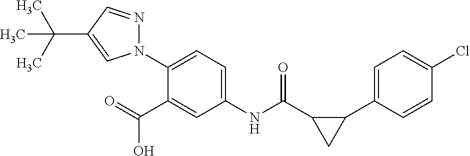 [SMILES: CC(C)(C)c1cnn(-c2ccc(NC(=O)C3CC3c3ccc(Cl)cc3)cc2C(=O)O)c1] | 2-(4-tert-Butyl- 1H-pyrazol-1- yl)-5-({[2-(4- chlorophenyl) cyclopropyl] carbonyl} amino)benzoic acid, as a mixture of trans enantiomers | ¹H NMR (500 MHz, DMSO-d6) δ [ppm] 12.84 (s, 1H), 10.54 (s, 1H), 7.94 (d, J = 2.4 Hz, 1H), 7.85-7.81 (m, 1H), 7.78 (dd, J = 8.7, 2.5 Hz, 1H), 7.59-7.55 (m, 1H), 7.48 (d, J = 8.7 Hz, 1H), 7.39-7.32 (m, 2H), 7.27-7.21 (m, 2H), 2.43 (ddd, J = 9.5, 6.3, 4.1 Hz, 1H), 2.11-2.02 (m, 1H), 1.58-1.48 (m, 1H), 1.41 (ddd, J = 8.2, 6.4, 4.3 Hz, 1H), 1.26 (s, 9H). LCMS (Analytical Method F): Rₜ = 3.79 min; MS (ESIPos) m/z = 438.1 (M + H)⁺. |
| 14 | 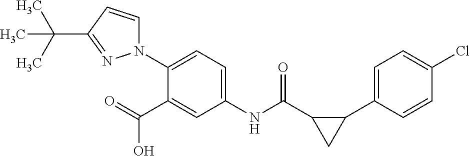 [SMILES: CC(C)(C)c1ccn(-c2ccc(NC(=O)C3CC3c3ccc(Cl)cc3)cc2C(=O)O)n1] | 2-(3-tert-Butyl- 1H-pyrazol-1- yl)-5-({[2-(4- chlorophenyl) cyclopropyl] carbonyl} amino)benzoic acid, as a mixture of trans enantiomers | ¹H NMR (500 MHz, DMSO-d6) δ [ppm] 12.81 (s, 1H), 10.53 (s, 1H), 7.95 (d, J = 2.4 Hz, 1H), 7.87 (d, J = 2.4 Hz, 1H), 7.76 (dd, J = 8.7, 2.4 Hz, 1H), 7.48 (d, J = 8.7 Hz, 1H), 7.39-7.32 (m, 2H), 7.27- 7.20 (m, 2H), 6.31 (d, J = 2.4 Hz, 1H), 2.43 (ddd, J = 9.4, 6.3, 4.1 Hz, 1H), 2.12- 2.02 (m, 1H), 1.58-1.48 (m, 1H), 1.40 (ddd, J = 8.1, 6.3, 4.2 Hz, 1H), 1.26 (s, 9H). LCMS (Analytical Method F): Rₜ = 3.84 min; MS (ESIPos) m/z = 438.1 (M + H)⁺. |
| 15 | 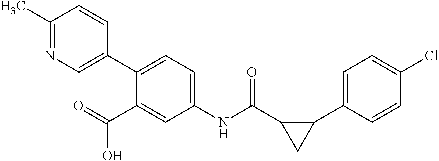 [SMILES: Cc1ccc(-c2ccc(NC(=O)C3CC3c3ccc(Cl)cc3)cc2C(=O)O)cn1] | 5-({[2-(4- chlorophenyl) cyclopropyl] carbonyl} amino)-2- (6- methylpyridin- 3-yl)benzoic acid, as a mixture of trans enantiomers | ¹H NMR (500 MHz, DMSO-d6) δ [ppm] 10.49 (s, 1H), 8.40 (s, 1H), 7.93 (s, 1H), 7.67 (dd, J = 36.4, 7.6 Hz, 2H), 7.42- 7.30 (m, 2H), 7.28-7.11 (m, 4H), 2.46 (s, 3H), 2.43-2.37 (m, 1H), 2.15-2.05 (m, 1H), 1.54-1.46 (m, 1H), 1.39-1.30 (m, 1H). LCMS (Analytical Method F): Rₜ = 2.35 mins; MS (ESIPos) m/z = 407.1 (M + H)⁺. |
| 16 | 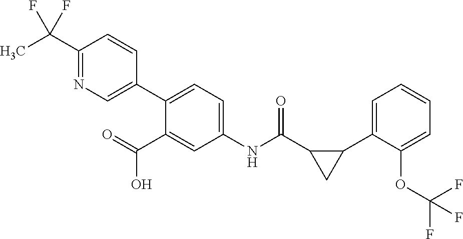 [SMILES: CC(F)(F)c1ccc(-c2ccc(NC(=O)C3CC3c3ccccc3OC(F)(F)F)cc2C(=O)O)cn1] | 2-[6-(1,1- difluoroethyl) pyridin-3-yl]-5- [({2-[2- (trifluoromethoxy) phenyl] cyclopropyl} carbonyl) amino]benzoic acid, as a mixture of trans enantiomers | ¹H NMR (500 MHz, DMSO-d6) δ [ppm] 10.57 (s, 1H), 8.63-8.49 (m, 1H), 8.19- 8.11 (m, 1H), 7.93-7.87 (m, 1H), 7.87- 7.81 (m, 1H), 7.75-7.68 (m, 1H), 7.42- 7.34 (m, 4H), 7.27-7.21 (m, 1H), 2.58- 2.52 (m, 1H), 2.12-1.98 (m, 4H), 1.58- 1.47 (m, 2H). LCMS (Analytical Method F): Rₜ = 3.72 min; MS (ESIPos) = 507 (M + H)⁺. |
| 17 | 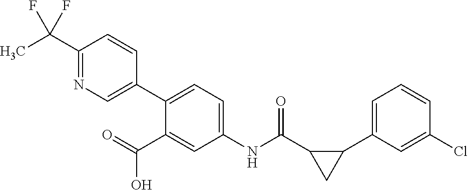 [SMILES: CC(F)(F)c1ccc(-c2ccc(NC(=O)C3CC3c3cccc(Cl)c3)cc2C(=O)O)cn1] | 5-({[(2-(3- chlorophenyl) cyclopropyl] carbonyl} amino)-2- [6-(1,1- difluoroethyl) pyridin-3- yl]benzoic acid, as a mixture of trans enantiomers | ¹H NMR (500 MHz, DMSO-d6) δ [ppm] 10.53-10.41 (m, 1H), 8.50 (d, J = 1.7 Hz, 1H), 8.14-8.01 (m, 1H), 7.82 (dd, J = 8.1, 2.3 Hz, 1H), 7.76 (dd, J = 8.4, 2.2 Hz, 1H), 7.64 (d, J = 8.1 Hz, 1H), 7.34- 7.16 (m, 4H), 7.16-7.09 (m, 1H), 2.40- 2.34 (m, 1H), 2.12-2.03 (m, 1H), 1.97 (t, J = 19.1 Hz, 3H), 1.51-1.42 (m, 1H), 1.42-1.33 (m, 1H). LCMS (Analytical Method F): Rₜ = 3.68 min; MS (ESIPos) m/z = 457 (M + H)⁺. |
| 18 | 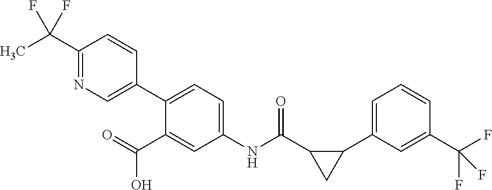 [SMILES: CC(F)(F)c1ccc(-c2ccc(NC(=O)C3CC3c3cccc(C(F)(F)F)c3)cc2C(=O)O)cn1] | 2-[6-(1,1- difluoroethyl) pyridin-3-yl]-5- [({2-[3- (trifluoromethyl) phenyl] cyclopropyl} carbonyl) amino]bentoic acid, as a mixture of trans enantiomers | ¹H NMR (500 MHz, DMSO-d6) δ [ppm] 10.56 (s, 1H), 8.62-8.51 (m, 1H), 8.17- 8.09 (m, 1H), 7.89 (dd, J = 8.1, 2.2 Hz, 1H), 7.86-7.79 (m, 1H), 7.70 (d, J = 8.1 Hz, 1H), 7.60-7.50 (m, 4H), 7.37 (d, J = 8.4 Hz, 1H), 2.60-2.53 (m, 1H), 2.22- 2.12 (m, 1H), 2.03 (t, J = 19.1 Hz, 3H), 1.62-1.55 (m, 1H), 1.55-1.48 (m, 1H). LCMS (Analytical Method F): Rₜ = 3.76 min; MS (ESIPos) m/z = 491 (M + H)⁺. |
| 19 | 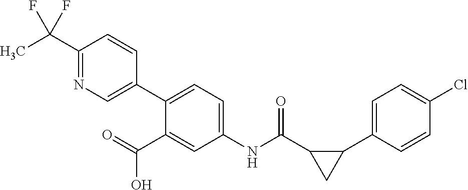 [SMILES: CC(F)(F)c1ccc(-c2ccc(NC(=O)C3CC3c3ccc(Cl)cc3)cc2C(=O)O)cn1] | 5-({[2-(4- chlorophenyl) cyclopropyl] carbonyl} amino)-2- [6-(1,1- difluoroethyl) pyridin-3- yl]benzoic acid, as a mixture of trans enantiomers | ¹H NMR (500 MHz, DMSO-d6) δ [ppm] 10.57 (s, 1H), 8.57 (d, J = 1.6 Hz, 1H), 8.17 (d, J = 2.1 Hz, 1H), 7.89 (dd, J = 8.1, 2.3 Hz, 1H), 7.84 (dd, J = 8.4, 2.3 Hz, 1H), 7.74-7.68 (m, 1H), 7.41-7.33 (m, 3H), 7.27-7.21 (m, 2H), 2.46-2.41 (m, 1H), 2.14-1.95 (m, 4H), 1.58-1.49 (m, 1H), 1.45-1.36 (m, 1H). LCMS (Analytical Method F): Rₜ = 3.67 min; MS (ESIPos) m/z = 457 (M + H)⁺. |
| 20 | 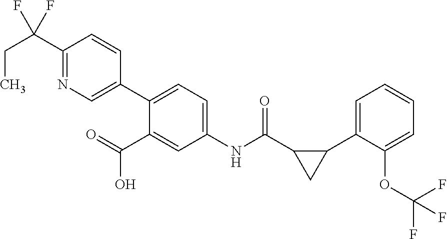 [SMILES: CCC(F)(F)c1ccc(-c2ccc(NC(=O)C3CC3c3ccccc3OC(F)(F)F)cc2C(=O)O)cn1] | 2-[6-(1,1- difluoropropyl) pyridin-3-yl]-5- [({2-[2- (trifluoromethoxy) phenyl] cyclopropyl} carbonyl) amino]benzoic acid, as a mixture of trans enantiomers | ¹H NMR (500 MHz, DMSO-d6) δ [ppm] 10.59 (s, 1H), 8.59 (d, J = 1.7 Hz, 1H), 8.17 (d, J = 1.9 Hz, 1H), 7.90 (dd, J = 8.1, 2.2 Hz, 1H), 7.85 (dd, J = 8.4, 2.2 Hz, 1H), 7.70 (d, J = 8.1 Hz, 1H), 7.43-7.35 (m, 4H), 7.27-7.22 (m, 1H), 2.58-2.54 (m, 1H), 2.44-2.28 (m, 2H), 2.14-2.06 (m, 1H), 1.60-1.48 (m, 2H), 0.96 (t, J = 7.5 Hz, 3H); CO₂H not observed. LCMS (Analytical Method F): Rₜ = 3.87 min; MS (ESIPos) m/z = 521.1 (M + H)⁺. |
| 21 | 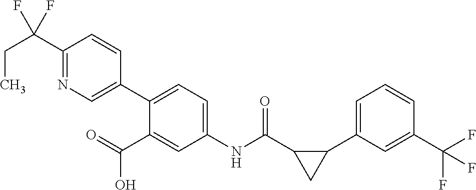 [SMILES: CCC(F)(F)c1ccc(-c2ccc(NC(=O)C3CC3c3cccc(C(F)(F)F)c3)cc2C(=O)O)cn1] | 2-[6-(1,1- difluoropropyl) pyridin-3-yl]-5- [({2-[3- (trifluoromethyl) phenyl] cyclopropyl} carbonyl) amino]benzoic acid, as a mixture of trans enantiomers | ¹H NMR (500 MHz, DMSO-d6) δ [ppm] 12.97 (s, 1H), 10.59 (s, 1H), 8.58 (d, J = 1.8 Hz, 1H), 8.19 (d, J = 2.2 Hz, 1H), 7.89 (dd, J = 8.1, 2.2 Hz, 1H), 7.86 (dd, J = 8.4, 2.3 Hz, 1H), 7.70 (d, J = 8.0 Hz, 1H), 7.61-7.52 (m, 4H), 7.41 (d, J = 8.4 Hz, 1H), 2.57 (ddd, J = 9.2, 6.3, 4.1 Hz, 1H), 2.44-2.28 (m, 2H), 2.22-2.16 (m, 1H), 1.62-1.57 (m, 1H), 1.54 (ddd, J = 8.2, 6.3, 4.4 Hz, 1H), 0.96 (t, J = 7.5 Hz, 3H). LCMS (Analytical Method F): Rₜ = 3.91 min; MS (ESIPos) m/z = 505.1 (M + H)⁺. |
| 22 | 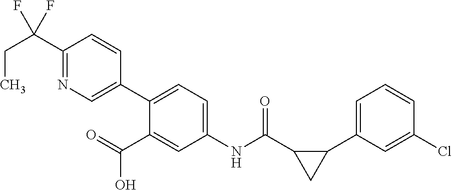 [SMILES: CCC(F)(F)c1ccc(-c2ccc(NC(=O)C3CC3c3cccc(Cl)c3)cc2C(=O)O)cn1] | 5-({[2-(3- chlorophenyl) cyclopropyl] carbonyl} amino)-2- [6-(1,1- difluoropropyl) pyridin-3- yl]benzoic acid, as a mixture of trans enantiomers | ¹H NMR (500 MHz, DMSO-d6) δ [ppm] 12.98 (s, 1H), 10.58 (s, 1H), 8.58 (d, J = 1.8 Hz, 1H), 8.19 (d, J = 2.2 Hz, 1H), 7.89 (dd, J = 8.1, 2.2 Hz, 1H), 7.86 (dd, J = 8.4, 2.2 Hz, 1H), 7.72-7.69 (m, 1H), 7.41 (d, J = 8.4 Hz, 1H), 7.36-7.25 (m, 3H), 7.23-7.18 (m, 1H), 2.46 (ddd, J = 9.4, 6.3, 4.1 Hz, 1H), 2.44-2.29 (m, 2H), 2.18-2.09 (m, 1H), 1.59-1.52 (m, 1H), 1.47 (ddd, J = 8.2, 6.3, 4.3 Hz, 1H), 0.96 (t, J = 7.5 Hz, 3H). LCMS (Analytical Method F): Rₜ = 3.84 min; MS (ESIPos) m/z = 471.1 (M + H)⁺. |
| 23 | 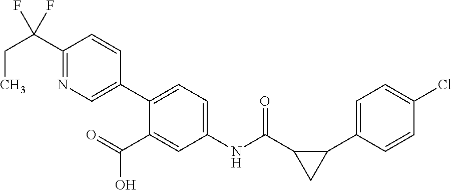 [SMILES: CCC(F)(F)c1ccc(-c2ccc(NC(=O)C3CC3c3ccc(Cl)cc3)cc2C(=O)O)cn1] | 5-({[2-(4- Chlorophenyl) cyclopropyl] carbonyl} amino)-2- [6-(1,1- difluoropropyl) pyridin-3- yl]benzoic acid, as a mixture of trans enantiomers | ¹H NMR (500 MHz, DMSO-d6) δ [ppm] 12.97 (s, 1H), 10.58 (s, 1H), 8.57 (d, J = 1.8 Hz, 1H), 8.19 (d, J = 2.2 Hz, 1H), 7.88 (dd, J = 8.1, 2.3 Hz, 1H), 7.86 (dd, J = 8.4, 2.3 Hz, 1H), 7.69 (d, J = 8.1 Hz, 1H), 7.40 (d, J = 8.4 Hz, 1H), 7.38-7.34 (m, 2H), 7.27-7.22 (m, 2H), 2.47-2.41 (m, 1H), 2.40-2.28 (m, 2H), 2.15-2.04 (m, 1H), 1.56-1.52 (m, 1H), 1.41 (ddd, J = 8.1, 6.3, 4.3 Hz, 1H), 1.00-0.90 (m, 3H). LCMS (Analytical Method F): Rₜ = 3.84 min; MS (ESIPos) m/z = 471.0 (M + H)⁺. |
| 24 | 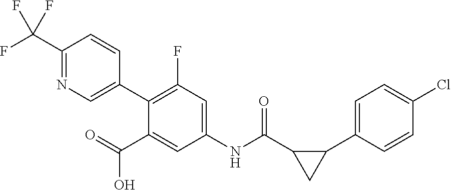 [SMILES: O=C(O)c1cc(NC(=O)C2CC2c2ccc(Cl)cc2)cc(F)c1-c1ccc(C(F)(F)F)nc1] | 5-({[2-(4- chlorophenyl) cyclopropyl] carbonyl} amino)-3- fluoro-2-[6- (trifluoromethyl) pyridin-3- yl]benzoic acid, as a mixture of trans enantiomers | ¹H NMR (500 MHz, DMSO-d6) δ 13.22 (s, 1H), 10.80 (s, 1H), 8.67 (s, 1H), 8.04 (d, J = 7.7 Hz, 1H), 7.97-7.90 (m, 3H), 7.37 (d, J = 8.0 Hz, 2H), 7.26 (d, J = 8.1 Hz, 2H), 2.13-2.05 (m, 1H), 1.59-1.53 (m, 1H), 1.48-1.41 (m, 1H)-missing signal under DMSO peak at 2.50 ppm. LCMS (Analytical Method D): Rₜ = 4.97 min; MS (ESIPos) m/z = 478.95 (M + H)⁺. |
| 25 | 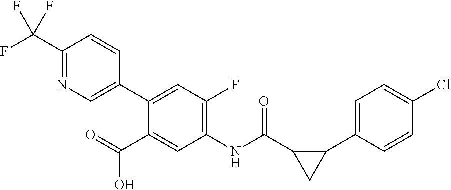 [SMILES: O=C(O)c1cc(NC(=O)C2CC2c2ccc(Cl)cc2)c(F)cc1-c1ccc(C(F)(F)F)nc1] | 5-({[2-(4- chlorophenyl) cyclopropyl] carbonyl} amino)-4- fluoro-2-[6- (trifluoromethyl) pyridin-3- yl]benzoic acid, as a mixture of trans enantiomers | ¹H NMR (500 MHz, DMSO-d6) δ [ppm] 13.14 (br s, 1H), 10.36 (s, 1H), 8.71- 8.70 (m, 2H), 8.05 (d, J = 8.0 Hz, 1H), 7.94 (d, J = 8.1 Hz, 1H), 7.46 (d, J = 11.3 Hz, 1H), 7.37 (d, J = 8.4 Hz, 2H), 7.25 (d, J = 8.4 Hz, 2H), 2.48-2.43 (m, 2H), 1.59-1.51 (m, 1H), 1.44-1.36 (m, 1H). LCMS (Analytical Method D): Rₜ = 4.88 min; MS (ESIPos) m/z = 478.95 (M + H)⁺. |

### BIOLOGICAL ASSAYS

Example compounds were tested in selected biological assays one or more times. When tested more than once, data are reported as either average values or as median values, wherein
The average value, also referred to as the arithmetic mean value, represents the sum of the values obtained divided by the number of times tested, and
The median value represents the middle number of the group of values when ranked in ascending or descending order. If the number of values in the data set is odd, the median is the middle value. If the number of values in the data set is even, the median is the arithmetic mean of the two middle values.

Example compounds were synthesised one or more times. When synthesised more than once, data from biological assays represent average values or median values calculated utilising data sets obtained from testing of one or more synthetic batch.

The potency to inhibit the Bradykinin B1 receptors was determined for the example compounds of this invention in a cell-based fluorescent calcium-mobilisation assay. The assay measures the ability of example compounds to inhibit Bradykinin B1 receptor agonist-induced increase of intracellular free Ca²⁺ in the cell line expressing B1 receptor. Specifically, calcium indicator—loaded cells are pre-incubated in the absence or presence of different concentrations of example compounds followed by the stimulation with a selective B1 receptor agonist peptide. The change of the intracellular Ca²⁺ concentration is monitored with a fluorescent plate reader FLIPR (Molecular Devices).

Calcium Flux Assays (FLIPR) with Cells Expressing Bradykinin B1 Receptor

Calcium flux Assay (FLIPR) with recombinant cells for Bradykinin B1 receptor antagonist, either in the presence (hB1 IC₅₀) or absence (hB1 free IC₅₀) of 0.1% BSA in assay buffer

CHO-K1 cell line expressing human B1 receptor was purchased from Euroscreen (Gosselies, Belgium, with reference name hB1-D1). The cells were grown in Nutrient Mixture Ham's F12 (Sigma) containing 10% Foetal bovine serum (Sigma) and 400 μg/mL G418 (Sigma), 5 μg/mL puromycin (Sigma).

Notably, example compounds were tested in the FLIPR assays either in the presence (hB1 IC₅₀) or absence (hB1 free IC₅₀) of 0.1% BSA in assay buffer, in order to assess the potency shifts due to serum protein binding of compounds.

For the calcium flux assay, 80% confluent cells were detached from the culture vessels with Versene (Gibco), and seeded into 384-well plates (Cell binding Surface; Corning, N.Y.; #3683) at a density of 15,000 cells per well. Cells were seeded in a volume of 50 μL in medium without antibiotics and incubated overnight in a humidified atmosphere with 5% CO₂ at 37° C. The following day, the medium was replaced with 20 μL of 5 μM Fluo-4AM dye (Molecular Probes) in assay buffer (2.5 mM probenicid, 1 mg/mL pluronic acid, 135 mM NaCl, 5 mM KCl, 1.8 mM CaCl, 1 mM MgCl₂, 10 mM HEPES, 5.6 mM glucose, and 0.05% gelatine, pH 7.4), which contains or lacks 0.1% BSA for determination of compound potency units as IC₅₀ or free IC₅₀, respectively. The calcium indicator loaded cells were incubated at 37° C. for 2 hrs. Extracellular dye was then removed and each well was filled with 45 μL of assay buffer. Cell plates were kept in dark until used. Example compounds were assayed at 8 concentrations in triplicate. Serial 10-fold dilutions in 100% DMSO were made at a 100-times higher concentration than the final concentration, and then diluted 1:10 in assay buffer. 5 μL of each diluted compound was added to the well of cell plates (yielding final concentration with 1% DMSO), and incubated for 30 min at 28° C. before the addition of B1 receptor agonist on the FLIPR instrument.

Agonist plates contained the agonist Lys-(Des-Arg)-Bradykinin (Bachem, Brackley) at 3.5×EC₉₀ in assay buffer with 1% DMSO. The addition of agonist 20 μl per well to the assay plate was carried out on the FLIPR instrument while continuously monitoring Ca²⁺-dependent fluorescence at 538 nm. A peptide antagonist Lys-(Des-Arg-Leu)-Bradykinin (Bachem, Brackley) at 20 □M was used to determine the full inhibition as control.

Peak fluorescence was used to determine the response to agonist obtained at each concentration of example compound by the following equation:

% Response=100*(RFU_{(example compound)}−RFU_{(control)}/(RFU_{(DMSO)}−RFU_{(control)})
Control=full inhibition by the peptide antagonist Lys-(Des-Arg-Leu)-Bradykinin at 20 μM

The response values were plotted against the logarithm of the compound concentrations. The compounds were tested in triplicates per plate and mean values were plotted in Excel XLfit to determine IC₅₀ values, percentage of maximal inhibition and the Hill slopes.

Calcium Flux Assay (FLIPR) with Human Fibroblasts for Bradykinin B1 Receptor Antagonist (hB1 IMR-90 IC₅₀)

The Calcium flux Assay was carried out utilising IMR-90 human foetal lung fibroblasts (American Type Culture Collection, Rockville, Md.; and Coriell Institute, Camden, N.J.), which express native human B1 receptors after induction with human IL-1□.

The fibroblasts were cultured in complete growth media comprised of Dulbecco's modified Eagle's medium (DMEM; Sigma) containing 10% foetal bovine serum, 4 mM L-glutamine, and 1% nonessential amino acids. The cells were maintained in a humidified atmosphere with 5% CO₂ at 37° C. and were sub-cultured at a ratio of 1:3, every other day.

For the assay, IMR-90 fibroblasts were harvested using TrypLE Express (GIBCO/Invitrogen) and seeded into 384-well plates (Corning Cellbinding Surface, Cat. 3683) at a density of 15000 cells/well. The following day, cells were treated with 0.35 ng/mL human IL-1□ in 10% FBS/MEM for 3 h to up-regulate B1 receptors. Induced cells were loaded with fluorescent calcium indicator by incubation with 2.5 μM Fluo-4/AM (Invitrogen) at 37° C., 5% CO₂ for 2 h in the presence of 2.5 mM probenecid in 1% FBS/MEM. Extracellular dye was removed by washing with assay buffer (2.5 mM probenecid and 0.1% BSA in 20 mM HEPES/HBSS without bicarbonate or phenol red, pH 7.5). Example compounds were assayed at 8 concentrations in triplicate. After addition of example compounds to the cell plate and incubation for 30 min at 28° C., the addition of B1 agonist Lys-(Des-Arg)-Bradykinin (Bachem, Brackley) at a final concentration of EC₉₀ was carried out on the FLIPR instrument while continuously monitoring Ca²⁺-dependent fluorescence at 538 nm. A peptide antagonist Lys-(Des-Arg-Leu)-Bradykinin (Bachem, Brackley) at 20 μM was used to determine the full inhibition as control. IC₅₀ values were determined by the same way described for the FLIPR assay with recombinant cells.

| | hB1 | hB1 | hB1 |
|---|---|---|---|
| Example | free IC₅₀ | IC₅₀ | IC₅₀ IMR90 |
| No | [nM] | [nM] | [nM] |
| 1 | 207 | 65 | |
| 2 | 22 | 78 | 66 |
| 3 | 10 | | 51 |
| 4 | 1180 | | |
| 5 | 110 | | |
| 6 | 272 | | |
| 7 | 117 | | |
| 8 | 1130 | | |
| 9 | 17 | | |
| 10 | 353 | | |
| 11 | 32 | | |
| 12 | 126 | | |
| 13 | 1760 | | 3390 |
| 14 | 2220 | | |
| 15 | 2420 | | 12000 |
| 16 | 2570 | | |
| 17 | 2470 | | |
| 18 | 2510 | | |
| 19 | 2490 | | |
| 20 | 1520 | | |
| 21 | 403 | | |
| 22 | 1190 | | |
| 23 | 309 | | 529 |
| 24 | 1080 | | 2330 |
| 25 | 1260 | | 4730 |

Inhibitory Activity on Bradykinin B1 Receptor Agonist-Induced Secretion of IL-6 and IL-8 in Human IMR-90 Cells

The effect of the compound examples on secretion of the cytokine IL-6 and IL-8 was investigated in the human foetal lung fibroblast IMR-90 cell line. Here the induction of the cytokine secretion was induced by the Bradykinin B1 receptor agonists Lys-[Des-Arg9]Bradykinin (CAS 71800-36-7, Tocris Bioscience) and Sar-[D-Phe8]-des-Arg9-Bradykinin (CAS 126959-88-4, Tocris Bioscience) leading to the activation of the Bradykinin B1-driven signalling pathway. The inhibitory activity of the tested compound examples on Bradykinin B1 receptor agonist-induced secretion of IL-6 and IL-8 is indicative for the compounds' prominent anti-inflammatory mode of action in kinin driven inflammation.

IMR-90 cells were cultured in Eagle's Minimum Essential Medium (EMEM) containing 2 mM L-glutamine, 1 g/L glucose, 1.5 g/L NaHCO₃, 1 mM sodium pyruvate and non-essential amino acids (ATCC, 30-2003™) supplemented with 10% FBS (Biochrom, 50615) and 50 U/mL Penicillin, 50 μg/mL Streptomycin (PAA, P11-010). The assay was performed in EMEM and a cell density of 5×10-4 IMR-90 cells/96-well. The compound examples were serially diluted in 100% DMSO and evaluated at 8 different concentrations within the range of 3 nM and 10 μM and a final DMSO concentration of 0.4%. The IMR-90 cells were incubated with the respective concentration of the compound for 30 min. The enhanced secretion of IL-6 and IL-8 was induced by the stimulation of these cells with 0.1 μM Lys-[Des-Arg9]Bradykinin (Tocris, catalogue no. 3225) and 0.1 μM Sar-[D-Phe8]-des-Arg9-Bradykinin (Tocris, catalogue no. 3230) for 5 hours at 37° C. and 5% CO₂. Further, cells were treated with Lys-[Des-Arg9]Bradykinin and Sar-[D-Phe8]-des-Arg9-Bradykinin as neutral control and with 0.1% DMSO as inhibitor control. The amount of IL-6 and IL-8 in the supernatant was determined using the Human ProInflammatory Panel II (4-Plex) (MSD, K15025B) according to manufacturer's instruction. Briefly, supernatants were added onto assay plates and incubated at room temperature for 1-2 h with vigorous shaking at 600 rpm. Detection antibodies were then added onto the supernatants and incubated at room temperature for an additional 1-2 h with vigorous shaking at 600 rpm. Plates were washed three times with phosphate-buffered saline (PBS; 137 mM NaCl, 2.7 mM KCl, 6.5 mM Na₂HP0₄, 1.7 mM KH₂P0₄) containing 0.05% Tween-20 (Bio-Rad, 161-0781) and electrochemiluminescence detected using the MSD Sector Imager 6000 plate reader. The cell viability was measured using the CellTiter-Glo Luminescent Assay (Promega, G7571) following the manufacturers protocol. Briefly, the CellTiter-Glo Reagent was diluted with PBS (1:1) and added directly to cells. After incubation and shaking for 10 minutes luminescent signal was measured which was proportional to the amount of ATP present.

The effect of the compound example on the amount of secreted cytokine was calculated as 100/(measured cytokine concentration of neutral control-measured cytokine concentration of inhibitor control)*(measured cytokine concentration of compound example dose-measured cytokine concentration of inhibitor control). IC₅₀ values were determined using 4-parameter-fit.

The cell viability is measured using the CellTiter-Glo Luminescent Assay (Promega, G7571) following the manufacturer's protocol. The homogeneous assay procedure involves adding the single reagent (CellTiter-Glo Reagent) directly to cells cultured in serum-supplemented medium. Cell washing, removal of medium and multiple pipetting steps were not required. The system is able to detect as few as 15 cells/well in a 384-well format in 10 minutes after adding reagent and mixing. The homogeneous add-mix-measure format results in cell lysis and generation of a luminescent signal proportional to the amount of ATP present. The amount of ATP is directly proportional to the number of cells present in culture. The CellTiter-Glo Assay generates a glow-type luminescent signal, which has a half-life generally greater than five hours, depending on cell type and medium used. The extended half-life eliminates the need to use reagent injectors and provides flexibility for continuous or batch mode processing of multiple plates. The unique homogeneous format avoids errors that may be introduced by other ATP measurement methods that require multiple steps.

The compound examples were tested in triplicates per plate and the inhibitory activity was determined as the relation between neutral and inhibitor control in percent. IC₅₀ values were calculated using the 4-parameter logistic model.

The compounds Examples 1 and 2 showed no effect on the cell viability of the stimulated IMR-90 cells. The effect on the secretion of IL-6 and IL-8 is shown in the table below:

| | IL-8 | IL-6 |
|---|---|---|
| | secretion | secretion |
| Example | IC₅₀ | IC₅₀ |
| 1 | 509 nM | 99 nM |
| 2 | 185 nM | 322 nM |

Rat CFA In Vivo Model

Male Sprague Dawley rats are used. Mechanical hyperalgesia is induced by injecting 25 μL of Complete Freund's Adjuvant (CFA) into the plantar surface of one hind paw. Mechanical hyperalgesia is measured using the Pressure Application Measurement apparatus (Ugo Basile, Gemonio, Italy). Briefly, a linearly increasing pressure is applied to an area of ˜50 mm² of the plantar side of the hind paw until a behavioural response (paw withdrawal) is observed or until the pressure reached 1000 gf. The pressure at which the behavioural response occurred is recorded as the “Paw Withdrawal Threshold” (PWT). Both CFA-injected and contralateral PWTs are determined for each rat, in each treatment group and at each time point of the studies. The compound examples are administered orally in a vehicle of dimethylsulfoxide (DMSO), Polyethylenglycol (PEG) and 2-hydroxypropyl-beta-cyclodextrin (HPCD) (v/v/v=3:20:77). Rats receive a first dose of 5 mL/kg bodyweight of compound example per kg body weight 1 hour before CFA injection and a second dose 24 hours after the CFA injection. Mechanical hyperalgesia testing is performed approximately 2 hours before CFA injection, then 2 and 4 hours after the second dose of compound example (i.e. 26 and 28 hours after CFA treatment). Data are expressed as the mean±S.D. Area Under the Curve (AUC) of PWTs (defined in table 3 as “AUC of Paw withdrawal threshold (AUC 0-4 hours) post-vehicle” with respect to vehicle group or “AUC of Paw withdrawal threshold (AUC 0-4 hours) post-drug” with respect to compound example). Data are analysed by performing a one-way ANOVA followed by a Dunnett's post hoc test. For p values less than 0.05 the results are deemed to be statistically significant.

1. A compound of general formula (I): 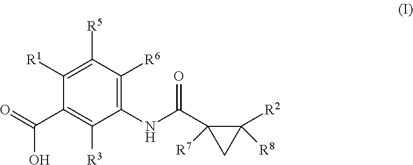
in which
R¹ represents
phenyl,
5- or 6-membered heteroaryl, wherein said 5-membered heteroaryl contains 1, 2 or 3 heteroatoms or heteroatom-containing groups independently selected from the group consisting of S, N, NH, and O, and wherein said 6-membered heteroaryl contains 1 or 2 nitrogen atoms, or
bicyclic 8- to 10-membered heteroaryl containing 1, 2 or 3 heteroatoms or heteroatom-containing groups independently selected from NH, N, O, S, SO and SO₂,
wherein said R¹ is optionally substituted at one or more carbon atoms with 1 to 3 substituents R^{1a} which are the same or different, wherein R^{1a} represents C₁-C₅-alkyl, C₃-C₇-cycloalkyl, —(C₁-C₃-alkyl)-(C₃-C₇-cycloalkyl), —OC₁-C₅-alkyl, —OC₃-C₇-cycloalkyl, NHR⁴, N(R⁴)₂, NH(C₃-C₇-cycloalkyl), halogen, CN, NHSO₂R⁴, SO₂R⁴, 5-to 7-membered lactam, or 4- to 7-membered heterocycloalkyl containing 1 or 2 heteroatoms or heteroatom-containing groups selected from NH, —NR⁴, N, O, S, SO and SO₂, and
wherein independently, if R¹ represents 5-membered heteroaryl or bicyclic 8- to 10-membered heteroaryl, each ring nitrogen atom, if present, of said R¹ is optionally substituted with a substituent R^{1b}, wherein R^{1b} represents C₁-C₅-alkyl, —(C₁-C₃-alkyl)-(C₃-C₇-cycloalkyl), C₃-C₇-cycloalkyl, SO₂R⁴, or 4- to 7-membered heterocycloalkyl containing 1 or 2 heteroatoms or heteroatom-containing groups selected from NH, —NR⁴, N, O, S, SO and SO₂, and
if R₁ₐ represents C₁-C₅-alkyl, C₃-C₇-cycloalkyl, —(C₁-C₃-alkyl)-(C₃-C₇-cycloalkyl), —OC₁-C₅-alkyl or —OC₃-C₇-cycloalkyl and/or if R^{1b} represents C₁-C₅-alkyl, —(C₁-C₃-alkyl)-(C₃-C₇-cycloalkyl) or C₃-C₇-cycloalkyl,
said C₁-C₅-alkyl, C₃-C₇-cycloalkyl, —(C₁-C₃-alkyl)-(C₃-C₇-cycloalkyl), —OC₁-C₅-alkyl and —OC₃-C₇-cycloalkyl independently are optionally substituted with one or more substituents independently selected from the group consisting of methyl, ethyl, OH, OR⁴ and F, and
if R^{1a} and/or R^{1b} represent 4- to 7-membered heterocycloalkyl, each carbon atom of said 4- to 7-membered heterocycloalkyl is optionally substituted with one or more substituents independently selected from the group consisting of OH, OR⁴ and F;
R² represents
—(CH₂)ₚ—(C₅-C₇-cycloalkyl),
—(CH₂)ₚ-phenyl,
5- or 6-membered heteroaryl wherein said 5-membered heteroaryl contains 1, 2 or 3 heteroatoms or heteroatom-containing groups independently selected from the group consisting of S, N, NH, and O, and wherein said 6-membered heteroaryl contains 1 or 2 N, or
bicyclic 8- to 10-membered heteroaryl, containing 1, 2 or 3 heteroatoms or heteroatom-containing groups independently selected from NH, N, O, S, SO and SO₂,
wherein said R² is optionally substituted at one or more carbon atoms with 1 to 3 substituents R^{2a} which are the same or different wherein R^{2a} represents C₁-C₅-alkyl, C₃-C₇-cycloalkyl, —(C₁-C₃-alkyl)-(C₃-C₇-cycloalkyl), —OC₁-C₅-alkyl, —OC₃-C₇-cycloalkyl, halogen, OH or CN, and
wherein independently, if R² represents 5-membered heteroaryl or bicyclic 8- to 10-membered heteroaryl, each ring nitrogen atom, if present, of said R² is optionally substituted with a substituent R^{2b} wherein R^{2b} represents C₁-C₅-alkyl, C₃-C₇-cycloalkyl or —(C₁-C₃-alkyl)-(C₃-C₇-cycloalkyl), and
if R^{2a} represents C₁-C₅-alkyl, C₃-C₇-cycloalkyl, —(C₁-C₃-alkyl)-(C₃-C₇-cycloalkyl), —OC₁-C₅-alkyl or —OC₃-C₇-cycloalkyl and/or if R^{2b} represents C₁-C₅-alkyl, C₃-C₇-cycloalkyl or —(C₁-C₃-alkyl)-(C₃-C₇-cycloalkyl),
said C₁-C₅-alkyl, C₃-C₇-cycloalkyl, —(C₁-C₃-alkyl)-(C₃-C₇-cycloalkyl), —OC₁-C₅-alkyl and —OC₃-C₇-cycloalkyl independently are optionally substituted with one or more substituents independently selected from the group consisting of OH, OR⁴, and 1 to 5 fluorine atoms;
p 0 or 1;
R³ represents H or F;
R⁴ represents C₁-C₅-alkyl, optionally substituted with 1 to 5 fluorine atoms;
R⁵ represents H, halogen, CN, C₁-C₅-alkyl, or —OC₁-C₅-alkyl wherein said C₁-C₅-alkyl and —OC₁-C₅-alkyl are optionally substituted with 1 to 5 fluorine atoms; and
R⁶ represents H, halogen, CN, OH, C₁-C₅-alkyl, or —OC₁-C₅-alkyl wherein said C₁-C₅-alkyl and —OC₁-C₅-alkyl are optionally substituted with 1 to 5 fluorine atoms; and
R⁷ and R⁸ independently represent H, or C₁-C₃-alkyl, wherein the C₁-C₃-alkyl is independently optionally substituted with 1 to 3 fluorine atoms;
or an isomer, enantiomer, diastereomer, racemate, hydrate, solvate, or a salt thereof, or a mixture of the same.

2.-14. (canceled)
